SEQUENCE LISTING (70 sequences)

SEQ ID NO: 1 - DNA, 7538 bp, synthetic construct
  source 1..7538: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtcttaagctggagtacggtggcatgatcttggctcactgcaacctctgcctcctgggttcaagcaattctcctgcctccttagtagctgggattacaggcgcctgccatcacgcccagctaatttttatatttttagtagagacggggtttcaccatgttggtcaggctggtctcaaactcctgacctcaagtgatctgcccaccttggcctcccaaagtgttgggattataggcctgtgccaccgcagctggccagacttgttatatggtgtcaagatgctccaagaatgagtgttctagcagtgcaaggtgaatgctatgtggccttttatgtcctatccttggaactcatatattccttcacatctcttttactctattctttaagacagtcacaaacctgcccagtttcaagggggagggatgtagacctcatttcttgagcagacaattattaaagaatttgcagccatgttttaaggctgccatatcttaaatgtatctatccatttttcccagttgctaagatcctttattttgtgtaagaatcttttcatggcaaattaaaaaaacaactcatgctagcctgggggaaaaaaggcatggataatttattataaggatgcagtgttatctcacagaacactcccaaaaatgtcacttaagtcctaatgtgtgctcagcgccagtgggtgctgaggggttagtgaggccaggtcttccctgctgatgcatcagcacacctgggcagtgatggaattctcgagtggtttgtctggtcaaccaccgcggtctcagtggtgtacggtacaaacccactcgagtacgtatcgatgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagcctgcaggctagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggctccggagctactaacttcagcctgctgaagcaggctggagacgtggaggagaaccctggacctatgaccgagtacaagcccacggtgcgcctcgccacccgcgacgacgtccccagggccgtgcgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgccggaagcggagagggcagaggaagtctgctaacatgcggtgacgtcgaggagaatcctggacctatggcctcctaccctggccatcagcacgcctccgccttcgaccaggccgccaggtccagaggccactccaaccggcggaccgccctgcggcctcggagacagcaggaggccaccgaagtccggcctgagcagaagatgcctaccctgctgcgggtgtacatcgacggccctcacggcatgggcaagaccaccaccacccagctgctggtcgccctgggctcccgggacgacatcgtgtacgtgcctgagcctatgacctactggcgggtgctgggcgcctccgagacaatcgccaacatctataccacccagcaccggctggaccagggcgagatctctgccggcgacgccgccgtggtgatgacctccgcccagatcaccatgggcatgccttacgccgtgaccgacgccgtgctggcccctcacatcggaggcgaggccggatcttctcacgcccctccccctgccctgaccctgatcttcgaccggcaccctatcgccgccctgctgtgctaccctgccgccagatacctgatgggctccatgacccctcaggctgtgctggccttcgtggccctgatccctcccaccctgcctggcaccaacatcgtgctgggagccctgcctgaggaccggcacatcgaccggctggccaagaggcagcggcctggcgagaggctggacctggccatgctggccgccatccggcgggtgtacggcctgctggccaacaccgtgcggtatctgcagtgcggcggctcctggagagaggactggggccagctgtccggcacagccgtgccacctcagggcgccgagcctcagtccaacgctggccctagaccacacatcggcgacaccctgtttaccctgttcagagcccctgagctgctggcccccaacggcgacctgtacaacgtgttcgcctgggctctggacgtgctggccaagcggctgagatccatgcacgtgttcatcctggactacgaccagtcccctgccggatgcagagatgccctgctgcagctgacctccggcatggtgcagacccacgtgaccacccctggctccatccctaccatctgcgacctggcccggaccttcgcccgggagatgggcgaggccaactaactagttcgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcagggcccgtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatccgttaacaccggtgtggtttgtctggtcaaccaccgcggactcagtggtgtacggtacaaacccaccggtaccctattgctccagaggcccttaggtgatgccactaatgggagggactggagaactgtggcaggcctgtcacagagcaactgaagaggccacagtatggagctgcttgtgccgctcccaggtggggactggaaagctggagatctagggagggctgtttggagcagtgttcttccatccctacacattccatccccacagaaggaattcccatttctttctagagggtaagcttctcagatctttcttcctcttctgtcaggctgcctctggccatcccagcctagtagatttagtcatccattggtttcttgagtgtgccttgcattgtgacagtgaacaagacattcaagatcccgagaagatggaaaaatgctctgacctgggatccagcagccctgaccctgtctgttactctccaagtgactttggacatatagtcaatcctctccctctgggcctcagtttccccatatgtcaaatcagggccttgaccttgaaggatctctaagatcccttcagaactcccagaagaaagatgattctggcatattgagaactcctaaaatttcaatcctcgacctcaagtttattttctgtcttctgccttactgctggctggcttactcttgctgtaatttatttaactgagccgtgctgagcagtttagaactgagtttgcatgaatcactgtccataaaatcgagttaagtggatttcattccacacggcgcacctgtgtatatacacaggaagcttcgtacggcggccgcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - DNA, 7538 bp, synthetic construct
  source 1..7538: mol_type = other DNA
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgtcttaagctccaggttcttgccgtaatgtttcagcttaaagaaaaccaacccatacttctttaggttgccctgaaaaggtgaatcttctgtgaagccttttctgtctttatctgttaaaattaattaagctcttgtttgtgctcccagaaaatcactttagaccttagaaagatggcagcctcagttttctcatttgtaatataggggtacccatagggtcattgtgagaaataatagaaataaatcatggaagtgcttggatagtagctggtacttagccctcatcagtcatcattgtcattgttgttaagagtaataataataataatatcacttattataattactcacctctaggtttttaaaaaacttacaggccgggcacattggctaacgcctgtaatcttagcactttgggaggccaaggcaggcagatcgcctgaggtcagcagttcaagaccagcttgggcaacatggtgaaaccctgtctctactaaaaatatgaaaattagccacgcgtggtggctcatgcctgtaatcccagctactcgggaggctgagagaggagaattgcttgaacccgggaggcggaggttgcagtgagccaagatggcactattgcactccagcctgggtgacgagaaactccatctcataaaaacaaaaacaaaaacaaaaaaacttacataaatttatggggaacaagtacaattttgtgacaagcgtaaattgcatagtcgtgaagtcagggcttctgaattctcgagtggtttgtctggtcaaccaccgcggtctcagtggtgtacggtacaaacccactcgagtacgtatcgatgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagcctgcaggctagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggctccggagctactaacttcagcctgctgaagcaggctggagacgtggaggagaaccctggacctatgaccgagtacaagcccacggtgcgcctcgccacccgcgacgacgtccccagggccgtgcgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgccggaagcggagagggcagaggaagtctgctaacatgcggtgacgtcgaggagaatcctggacctatggcctcctaccctggccatcagcacgcctccgccttcgaccaggccgccaggtccagaggccactccaaccggcggaccgccctgcggcctcggagacagcaggaggccaccgaagtccggcctgagcagaagatgcctaccctgctgcgggtgtacatcgacggccctcacggcatgggcaagaccaccaccacccagctgctggtcgccctgggctcccgggacgacatcgtgtacgtgcctgagcctatgacctactggcgggtgctgggcgcctccgagacaatcgccaacatctataccacccagcaccggctggaccagggcgagatctctgccggcgacgccgccgtggtgatgacctccgcccagatcaccatgggcatgccttacgccgtgaccgacgccgtgctggcccctcacatcggaggcgaggccggatcttctcacgcccctccccctgccctgaccctgatcttcgaccggcaccctatcgccgccctgctgtgctaccctgccgccagatacctgatgggctccatgacccctcaggctgtgctggccttcgtggccctgatccctcccaccctgcctggcaccaacatcgtgctgggagccctgcctgaggaccggcacatcgaccggctggccaagaggcagcggcctggcgagaggctggacctggccatgctggccgccatccggcgggtgtacggcctgctggccaacaccgtgcggtatctgcagtgcggcggctcctggagagaggactggggccagctgtccggcacagccgtgccacctcagggcgccgagcctcagtccaacgctggccctagaccacacatcggcgacaccctgtttaccctgttcagagcccctgagctgctggcccccaacggcgacctgtacaacgtgttcgcctgggctctggacgtgctggccaagcggctgagatccatgcacgtgttcatcctggactacgaccagtcccctgccggatgcagagatgccctgctgcagctgacctccggcatggtgcagacccacgtgaccacccctggctccatccctaccatctgcgacctggcccggaccttcgcccgggagatgggcgaggccaactaactagttcgaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcagggcccgtttaaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggatccgttaacaccggtgtggtttgtctggtcaaccaccgcggactcagtggtgtacggtacaaacccaccggtaccaaggtatctatcaccagattgatatctattggtatctatcaccagattgatatctattgatatctatcaccagattgatatcaccacgttgtacctattaattaatttctcatcatcctccccccacccttctgagtctccagtgtatattattccataccctaatgccatgtgttcacattatttagctcccacttatgagtgaacaatgtgatatttgtctttcagtgtctgacttttttaacttaagataacagcttccagttctacccatgtggctgcgagagacatgatttcattcttttttatggctgaataatattccattgcatatatatacaccacagttttaaatccaatcatccattgatggacacttaggttgattccacatctttgctattgtgactagtgctacgataaacatacaagtgcaggtatctaattatttcttttcctttggatggatacccagtagtgggattgctggattgaatggtagttctaattttagttctctgacaaatctccatactgttcttcctagagattgtactaatttacattcccaccaacagcgtataagagtttccttttctccacatcgccactaggttgtaagttccttgaatgtagggatcatgtctcttttcttcattgttttataacaagagcctaggagtgtttggcacatggcaagtacttgttttctagctgacagatgaatgaacaaatgagtgaaggatgcttaagcttcgtacggcggccgcacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 3 - DNA, 380 bp, synthetic construct
  source 1..380: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatg

SEQ ID NO: 4 - DNA, 204 bp, synthetic construct
  source 1..204: mol_type = other DNA
gtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 5 - DNA, 347 bp, synthetic construct
  source 1..347: mol_type = other DNA
gaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 6 - DNA, 1131 bp, synthetic construct
  source 1..1131: mol_type = other DNA
atggcctcctaccctggccatcagcacgcctccgccttcgaccaggccgccaggtccagaggccactccaaccggcggaccgccctgcggcctcggagacagcaggaggccaccgaagtccggcctgagcagaagatgcctaccctgctgcgggtgtacatcgacggccctcacggcatgggcaagaccaccaccacccagctgctggtcgccctgggctcccgggacgacatcgtgtacgtgcctgagcctatgacctactggcgggtgctgggcgcctccgagacaatcgccaacatctataccacccagcaccggctggaccagggcgagatctctgccggcgacgccgccgtggtgatgacctccgcccagatcaccatgggcatgccttacgccgtgaccgacgccgtgctggcccctcacatcggaggcgaggccggatcttctcacgcccctccccctgccctgaccctgatcttcgaccggcaccctatcgccgccctgctgtgctaccctgccgccagatacctgatgggctccatgacccctcaggctgtgctggccttcgtggccctgatccctcccaccctgcctggcaccaacatcgtgctgggagccctgcctgaggaccggcacatcgaccggctggccaagaggcagcggcctggcgagaggctggacctggccatgctggccgccatccggcgggtgtacggcctgctggccaacaccgtgcggtatctgcagtgcggcggctcctggagagaggactggggccagctgtccggcacagccgtgccacctcagggcgccgagcctcagtccaacgctggccctagaccacacatcggcgacaccctgtttaccctgttcagagcccctgagctgctggcccccaacggcgacctgtacaacgtgttcgcctgggctctggacgtgctggccaagcggctgagatccatgcacgtgttcatcctggactacgaccagtcccctgccggatgcagagatgccctgctgcagctgacctccggcatggtgcagacccacgtgaccacccctggctccatccctaccatctgcgacctggcccggaccttcgcccgggagatgggcgaggccaactaa

SEQ ID NO: 7 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

SEQ ID NO: 8 - DNA, 597 bp, synthetic construct
  source 1..597: mol_type = other DNA
atgaccgagtacaagcccacggtgcgcctcgccacccgcgacgacgtccccagggccgtgcgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 9 - protein, 22 aa, synthetic construct
GSGATNFSLLKQAGDVEENPGP

SEQ ID NO: 10 - DNA, 57 bp, synthetic construct
  source 1..57: mol_type = other DNA
gctactaacttcagcctgctgaagcaggctggagacgtggaggagaaccctggacct

SEQ ID NO: 11 - DNA, 66 bp, synthetic construct
  source 1..66: mol_type = other DNA
ggaagcggagctactaacttcagcctgctgaagcaggctggagacgtggaggagaaccctggacct

SEQ ID NO: 12 - DNA, 66 bp, synthetic construct
  source 1..66: mol_type = other DNA
ggcagcggcgccacaaacttctctctgctaaagcaagcaggtgatgttgaagaaaaccccgggcct

SEQ ID NO: 13 - DNA, 63 bp, synthetic construct
  source 1..63: mol_type = other DNA
ggctccggcgagggcaggggaagtcttctaacatgcggggacgtggaggaaaatcccggccca

SEQ ID NO: 14 - protein, 21 aa, synthetic construct
GSGEGRGSLLTCGDVEENPGP

SEQ ID NO: 15 - DNA, 54 bp, synthetic construct
  source 1..54: mol_type = other DNA
gagggcagaggaagtctgctaacatgcggtgacgtcgaggagaatcctggacct

SEQ ID NO: 16 - DNA, 63 bp, synthetic construct
  source 1..63: mol_type = other DNA
ggaagcggagagggcagaggaagtctgctaacatgcggtgacgtcgaggagaatcctggacct

SEQ ID NO: 17 - DNA, 345 bp, synthetic construct
  source 1..345: mol_type = other DNA
ctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctc

SEQ ID NO: 18 - DNA, 131 bp, synthetic construct
  source 1..131: mol_type = other DNA
aacttgtttattgcagcttataatggttacaaataaagcaatagcatcacaaatttcacaaataaagcatttttttcactgcattctagttgtggtttgtccaaactcatcaatgtatcttatcatgtctg

SEQ ID NO: 19 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 20 - DNA, 590 bp, synthetic construct
  source 1..590: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgttcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgca

SEQ ID NO: 21 - RNA, 20 nt, synthetic construct
  source 1..20: mol_type = other RNA
tcgtgaagtcagggcttcta

SEQ ID NO: 22 - RNA, 20 nt, synthetic construct
  source 1..20: mol_type = other RNA
gagcaatagcatcactgccc

SEQ ID NO: 23 - RNA, 20 nt, synthetic construct
  source 1..20: mol_type = other RNA
cagtgatgctattgctccag

SEQ ID NO: 24 - DNA, 750 bp, synthetic construct
  source 1..750: mol_type = other DNA
ctccaggttcttgccgtaatgtttcagcttaaagaaaaccaacccatacttctttaggttgccctgaaaaggtgaatcttctgtgaagccttttctgtctttatctgttaaaattaattaagctcttgtttgtgctcccagaaaatcactttagaccttagaaagatggcagcctcagttttctcatttgtaatataggggtacccatagggtcattgtgagaaataatagaaataaatcatggaagtgcttggatagtagctggtacttagccctcatcagtcatcattgtcattgttgttaagagtaataataataataatatcacttattataattactcacctctaggtttttaaaaaacttacaggccgggcacattggctaacgcctgtaatcttagcactttgggaggccaaggcaggcagatcgcctgaggtcagcagttcaagaccagcttgggcaacatggtgaaaccctgtctctactaaaaatatgaaaattagccacgcgtggtggctcatgcctgtaatcccagctactcgggaggctgagagaggagaattgcttgaacccgggaggcggaggttgcagtgagccaagatggcactattgcactccagcctgggtgacgagaaactccatctcataaaaacaaaaacaaaaacaaaaaaacttacataaatttatggggaacaagtacaattttgtgacaagcgtaaattgcatagtcgtgaagtcagggcttct

SEQ ID NO: 25 - DNA, 750 bp, synthetic construct
  source 1..750: mol_type = other DNA
aaggtatctatcaccagattgatatctattggtatctatcaccagattgatatctattgatatctatcaccagattgatatcaccacgttgtacctattaattaatttctcatcatcctccccccacccttctgagtctccagtgtatattattccataccctaatgccatgtgttcacattatttagctcccacttatgagtgaacaatgtgatatttgtctttcagtgtctgacttttttaacttaagataacagcttccagttctacccatgtggctgcgagagacatgatttcattcttttttatggctgaataatattccattgcatatatatacaccacagttttaaatccaatcatccattgatggacacttaggttgattccacatctttgctattgtgactagtgctacgataaacatacaagtgcaggtatctaattatttcttttcctttggatggatacccagtagtgggattgctggattgaatggtagttctaattttagttctctgacaaatctccatactgttcttcctagagattgtactaatttacattcccaccaacagcgtataagagtttccttttctccacatcgccactaggttgtaagttccttgaatgtagggatcatgtctcttttcttcattgttttataacaagagcctaggagtgtttggcacatggcaagtacttgttttctagctgacagatgaatgaacaaatgagtgaaggatgctt

SEQ ID NO: 26 - DNA, 750 bp, synthetic construct
  source 1..750: mol_type = other DNA
ctggagtacggtggcatgatcttggctcactgcaacctctgcctcctgggttcaagcaattctcctgcctccttagtagctgggattacaggcgcctgccatcacgcccagctaatttttatatttttagtagagacggggtttcaccatgttggtcaggctggtctcaaactcctgacctcaagtgatctgcccaccttggcctcccaaagtgttgggattataggcctgtgccaccgcagctggccagacttgttatatggtgtcaagatgctccaagaatgagtgttctagcagtgcaaggtgaatgctatgtggccttttatgtcctatccttggaactcatatattccttcacatctcttttactctattctttaagacagtcacaaacctgcccagtttcaagggggagggatgtagacctcatttcttgagcagacaattattaaagaatttgcagccatgttttaaggctgccatatcttaaatgtatctatccatttttcccagttgctaagatcctttattttgtgtaagaatcttttcatggcaaattaaaaaaacaactcatgctagcctgggggaaaaaaggcatggataatttattataaggatgcagtgttatctcacagaacactcccaaaaatgtcacttaagtcctaatgtgtgctcagcgccagtgggtgctgaggggttagtgaggccaggtcttccctgctgatgcatcagcacacctgggcagtgatg

SEQ ID NO: 27 - DNA, 750 bp, synthetic construct
  source 1..750: mol_type = other DNA
ctattgctccagaggcccttaggtgatgccactaatgggagggactggagaactgtggcaggcctgtcacagagcaactgaagaggccacagtatggagctgcttgtgccgctcccaggtggggactggaaagctggagatctagggagggctgtttggagcagtgttcttccatccctacacattccatccccacagaaggaattcccatttctttctagagggtaagcttctcagatctttcttcctcttctgtcaggctgcctctggccatcccagcctagtagatttagtcatccattggtttcttgagtgtgccttgcattgtgacagtgaacaagacattcaagatcccgagaagatggaaaaatgctctgacctgggatccagcagccctgaccctgtctgttactctccaagtgactttggacatatagtcaatcctctccctctgggcctcagtttccccatatgtcaaatcagggccttgaccttgaaggatctctaagatcccttcagaactcccagaagaaagatgattctggcatattgagaactcctaaaatttcaatcctcgacctcaagtttattttctgtcttctgccttactgctggctggcttactcttgctgtaatttatttaactgagccgtgctgagcagtttagaactgagtttgcatgaatcactgtccataaaatcgagttaagtggatttcattccacacggcgcacctgtgtatatacacagg

SEQ ID NO: 28 - DNA, 665 bp, synthetic construct
  source 1..665: mol_type = other DNA
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagccagcagacaccaggggggatacggggaaaaagcttgatatcatgtgtctgagcctgcatgtttgatggtgtcaggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgaggatggggct

SEQ ID NO: 29 - DNA, 665 bp, synthetic construct
  source 1..665: mol_type = other DNA
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagcttgatatcatgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgaggatggggct

SEQ ID NO: 30 - DNA, 414 bp, synthetic construct
  source 1..414: mol_type = other DNA
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacaca

SEQ ID NO: 31 - DNA, 244 bp, synthetic construct
  source 1..244: mol_type = other DNA
aagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctc

SEQ ID NO: 32 - DNA, 238 bp, synthetic construct
  source 1..238: mol_type = other DNA
ctgggtggagggagagaccaggggcaggtgaggaaaggcagggcccccagaatccctccatgcctgcccctcagtctccaggacttatgtgcaggtaccgtttggagctgtggtgcagttcccagtctcaccaccagatggcaccatgcccctgcagaagcagtgcccagagcaggccaggtggttctcgggggctgcggtggaggaatccacccagccgaagctctggcagggaagg

SEQ ID NO: 33 - DNA, 300 bp, synthetic construct
  source 1..300: mol_type = other DNA
ccaatcgtggcatatcctctaaactttcttttcccttcataaatcctctttcttttttttccccctcacagttttcctgaacaggttgactattaattgtgtctgcttgatgtggacaccaggtggcgctggacatcagatttggagaggcagttgtctagggaaccgggctctgtgccagcgcaggaggcaggctggctctcctattccagggatgctcatccaggaaggaaaggttgcatgctggacacactaaccttgaagaattcttctgtctctctcgtcatttagaaaggaagg

SEQ ID NO: 34 - DNA, 266 bp, synthetic construct
  source 1..266: mol_type = other DNA
agagcgagattccgtctcaaagaaaaaaaaagtaatgaaatgaataaaatgagtcctagagccagtaaatgtcgtaaatgtctcagctagtcaggtagtaaaaggtctcaactaggcagtggcagagcaggattcaaattcagggctgttgtgatgcctccgcagactctgagcgccacctggtggtaatttgtctgtgcctcttctgacgtggaagaacagcaactaacacactaacacggcatttactatgggccagccattgt

SEQ ID NO: 35 - DNA, 36 bp, synthetic construct
  source 1..36: mol_type = other DNA
aagggagacatctagtgatataagtgtgaactacac

SEQ ID NO: 36 - DNA, 77 bp, synthetic construct
  source 1..77: mol_type = other DNA
cccagggatgtacgtccctaacccgctagggggcagcacccaggcctgcactgccgcctgccggcaggggtccagtc

SEQ ID NO: 37 - DNA, 458 bp, synthetic construct
  source 1..458: mol_type = other DNA
gcttcttggaggtgtgaccatcgctcgaggtggtgtactgcccaacatccaggctgtgctgcttcccaagaagaccggcaaatcaagctaaaggttttgcactcgcaaacctcaacacctcaacggcccttatcagggccaccaaatatacaagaaagaataaagtctctgtaattcataatagtctctgtaattcataataaactcctactgcaactacaactcagacgcaaccccctctcctcctcctcctccacccctcctcccccccccctctctctctctctctctctctctctctgtcttccccattccggttttgtagttgttgtcgatcatccgatctaatatgcctatgcgaatacaatcacaaatgttattattataaacattctgttctgttctgttattgcttattgcttacacggatgaataaaagatactctgtgtcgcacagt

SEQ ID NO: 38 - DNA, 1211 bp, synthetic construct
  source 1..1211: mol_type = other DNA
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggatggggcatccacagcctccttgggcaacctgttcagtgcgtcaccaccctctgggggaaaaactgcctcctcatatccaacccaaacctcccctgtctcagtgtaaagccattcccccttgtcctatcaagggggagtttgctgtgacattgttggtctggggtgacacatgtttgccaattcagtgcatcacggagaggcagatcttggggataaggaagtgcaggacagcatggacgtgggacatgcaggtgttgagggctctgggacactctccaagtcacagcgttcagaacagccttaaggataagaagataggatagaaggacaaagagcaagttaaaacccagcatggagaggagcacaaaaaggccacagacactgctggtccctgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgagaatatt

SEQ ID NO: 39 - DNA, 353 bp, synthetic construct
  source 1..353: mol_type = other DNA
ggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 40 - DNA, 696 bp, synthetic construct
  source 1..696: mol_type = other DNA
atggtgagcaagggcgaggccgtgatcaaggagttcatgaggttcaaggtgcacatggagggctccatgaacggtcacgagttcgagatcgagggcgaaggcgagggcaggccctacgagggcacccagaccgccaagctgaaggtgacaaagggcggccccctgcccttctcctgggacatcctgtcccctcagttcatgtacggctccagggccttcatcaagcaccccgccgacatccccgactactacaagcagtccttccccgagggcttcaagtgggagagggtgatgaacttcgaggacggcggcgccgtgaccgtgacccaggacacctccctggaggacggcaccctgatctacaaggtgaagctgaggggcaccaacttccctcctgacggccccgtgatgcagaagaagacaatgggctgggaggcctccaccgagaggctgtaccccgaggacggcgtgctgaagggcgacatcaagatggccctgaggctgaaggacggcggcagatacctggccgacttcaagaccacctacaaggccaagaagcccgtgcagatgcccggcgcctacaacgtggacaggaagctggacatcacctcccacaacgaggactacaccgtggtggagcagtacgagaggtccgagggcaggcactccaccggcggcatggacgagctgtataag

SEQ ID NO: 41 - DNA, 589 bp, synthetic construct
  source 1..589: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgc

SEQ ID NO: 42 - DNA, 768 bp, synthetic construct
  source 1..768: mol_type = other DNA
aggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacc

SEQ ID NO: 43 - DNA, 477 bp, synthetic construct
  source 1..477: mol_type = other DNA
aattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccata

SEQ ID NO: 44 - DNA, 96 bp, synthetic construct
  source 1..96: mol_type = other DNA
ggtctctctggttagaccagatctgagcctgggagctctctggctaactagggaacccactgcttaagcctcaataaagcttgccttgagtgcttc

SEQ ID NO: 45 - DNA, 85 bp, synthetic construct
  source 1..85: mol_type = other DNA
aagtagtgtgtgcccgtctgttgtgtgactctggtaactagagatccctcagacccttttagtcagtgtggaaaatctctagcag

SEQ ID NO: 46 - DNA, 19 bp, synthetic construct
  source 1..19: mol_type = other DNA
ctgctttttgcctgtactg

SEQ ID NO: 47 - DNA, 360 bp, synthetic construct
  source 1..360: mol_type = other DNA
atgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcag

SEQ ID NO: 48 - DNA, 8141 bp, synthetic construct
  source 1..8141: mol_type = other DNA
ccggatgatcctgacgacggagtccgccgtcgtcgacaagccggcctggaagggctaattcactcccaaagaagacaagatagccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacatgatatcaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatactggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcaggatcttcagacctggaaattccctacaatccccaaagtcaaggagtagtagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagaaatccactttggaaaggaccagcaaagctcctctggaaaggtgaaggggcagtagtaatacaagataatagtgacataaaagtagtgccaagaagaaaagcaaagatcattagggattatggaaaacagatggcaggtgatgattgtgtggcaagtagacaggatgaggattagaacatggaaaagtttagtaaaacaccataaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggaccgagctcaagcttcgaagcgatcgcacgcgtggatccgaacagagagacagcagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatcggcgcgccaattcaagcgagaagacaagggcagccgccaccatggtgagcaagggcgaggccgtgatcaaggagttcatgaggttcaaggtgcacatggagggctccatgaacggtcacgagttcgagatcgagggcgaaggcgagggcaggccctacgagggcacccagaccgccaagctgaaggtgacaaagggcggccccctgcccttctcctgggacatcctgtcccctcagttcatgtacggctccagggccttcatcaagcaccccgccgacatccccgactactacaagcagtccttccccgagggcttcaagtgggagagggtgatgaacttcgaggacggcggcgccgtgaccgtgacccaggacacctccctggaggacggcaccctgatctacaaggtgaagctgaggggcaccaacttccctcctgacggccccgtgatgcagaagaagacaatgggctgggaggcctccaccgagaggctgtaccccgaggacggcgtgctgaagggcgacatcaagatggccctgaggctgaaggacggcggcagatacctggccgacttcaagaccacctacaaggccaagaagcccgtgcagatgcccggcgcctacaacgtggacaggaagctggacatcacctcccacaacgaggactacaccgtggtggagcagtacgagaggtccgagggcaggcactccaccggcggcatggacgagctgtataagtgactagtaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcgcggccgcatcgatgccgtagtacctttaagaccaatgacttacaaggcagctgtagatcttagccactttttaaaagaaaaggggggactggaagggctaattcactcccaaagaagacaagatagccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacatgatatcaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctccccagatctgctttttgcctgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtccggatgatcctgacgacggagaccgccgtcgtcgacaagccggcctcgaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattc

SEQ ID NO: 49 - DNA, 260 bp, synthetic construct
  source 1..260: mol_type = other DNA
ctcccgtgtggtacctgaggccggctcctgtggctctgagggggtctgcagcacccccttacatctgtccacagaagggctggggagcagctttcctgtccctcctgtgagtggccaccagggggagcgtggacacagctgcccgtgcagtgaccacctgccccccactcccgctactccagcagcagcggctccagccctggacaccctccctgcccccaccagcctggtcctgagccaggtgacctcctccagcatcc

SEQ ID NO: 50 - DNA, 325 bp, synthetic construct
  source 1..325: mol_type = other DNA
gtctgaatggtggccgtagtttgcagagccctggtttcttcttgcctctcagcttccaacttccccgtgagtgcctgctccttgatggactggactctaagcccttctttgcagcaagcacgatatcaagctttgtcagtagagggcgccggagggacactgtggaggaaggggccttttcatggtccacagagctctgttgtgcaatttcttgttcctgttgcatcttctcttagggtatgaacgcggggggacatcctctggggcttttcctcagctgtgcacccagaatgcatggtccctcgaccacctcatagcccatcct

SEQ ID NO: 51 - DNA, 52 bp, synthetic construct
  source 1..52: mol_type = other DNA
aggatatgcccttgactatttgtccgacatagtcaagggcatatcctttttt

SEQ ID NO: 52 - DNA, 46 bp, synthetic construct
  source 1..46: mol_type = other DNA
aggatatgcccttgactatttgtccgacatagtcaagggcatatcc

SEQ ID NO: 53 - DNA, 248 bp, synthetic construct
  source 1..248: mol_type = other DNA
tcgacgtgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagtaacttgacctaagtgtaaagttgagatttccttcaggtttatatagcttgtgcgccgcctgggtacctc

SEQ ID NO: 54 - DNA, 243 bp, synthetic construct
  source 1..243: mol_type = other DNA
ctgcagtatttagcatgccccacccatctgcaaggcattctggatagtgtcaaaacagccggaaatcaagtccgtttatctcaaactttagcattttgggaataaatgatatttgctatgctggttaaattagattttagttaaatttcctgctgaagctctagtacgataagcaacttgacctaagtgtaaagttgagatttccttcaggtttatatagcttgtgcgccgcctgggtacctc

SEQ ID NO: 55 - DNA, 225 bp, synthetic construct; identical to SEQ ID NO: 19
  source 1..225: mol_type = other DNA

SEQ ID NO: 56 - DNA, 665 bp, synthetic construct
  source 1..665: mol_type = other DNA
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatcctgacaccatcaaacatgcaggctcagacacatgatatcaagctttttccccgtatcccccctggtgtctgctggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctc

SEQ ID NO: 57 - DNA, 665 bp, synthetic construct
  source 1..665: mol_type = other DNA
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacatgatatcaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctc

SEQ ID NO: 58 - DNA, 414 bp, synthetic construct
  source 1..414: mol_type = other DNA
tgtgtctgagcctgcatgtttgatggtgtctggatgcaagcagaaggggtggaagagcttgcctggagagatacagctgggtcagtaggactgggacaggcagctggagaattgccatgtagatgttcatacaatcgtcaaatcatgaaggctggaaaagccctccaagatccccaagaccaaccccaacccacccaccgtgcccactggccatgtccctcagtgccacatccccacagttcttcatcacctccagggacggtgacccccccacctccgtgggcagctgtgccactgcagcaccgctctttggagaaggtaaatcttgctaaatccagcccgaccctcccctggcacaacgtaaggccattatctctcatccaactccaggacggagtcagtgaggatggggct

SEQ ID NO: 59 - DNA, 244 bp, synthetic construct
  source 1..244: mol_type = other DNA
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctt

SEQ ID NO: 60 - DNA, 238 bp, synthetic construct
  source 1..238: mol_type = other DNA
ccttccctgccagagcttcggctgggtggattcctccaccgcagcccccgagaaccacctggcctgctctgggcactgcttctgcaggggcatggtgccatctggtggtgagactgggaactgcaccacagctccaaacggtacctgcacataagtcctggagactgaggggcaggcatggagggattctgggggccctgcctttcctcacctgcccctggtctctccctccacccag

SEQ ID NO: 61 - DNA, 300 bp, synthetic construct
  source 1..300: mol_type = other DNA
ccttcctttctaaatgacgagagagacagaagaattcttcaaggttagtgtgtccagcatgcaacctttccttcctggatgagcatccctggaataggagagccagcctgcctcctgcgctggcacagagcccggttccctagacaactgcctctccaaatctgatgtccagcgccacctggtgtccacatcaagcagacacaattaatagtcaacctgttcaggaaaactgtgagggggaaaaaaaagaaagaggatttatgaagggaaaagaaagtttagaggatatgccacgattgg

SEQ ID NO: 62 - DNA, 266 bp, synthetic construct
  source 1..266: mol_type = other DNA
acaatggctggcccatagtaaatgccgtgttagtgtgttagttgctgttcttccacgtcagaagaggcacagacaaattaccaccaggtggcgctcagagtctgcggaggcatcacaacagccctgaatttgaatcctgctctgccactgcctagttgagaccttttactacctgactagctgagacatttacgacatttactggctctaggactcattttattcatttcattactttttttttctttgagacggaatctcgctct

SEQ ID NO: 63 - DNA, 36 bp, synthetic construct
  source 1..36: mol_type = other DNA
gtgtagttcacacttatatcactagatgtctccctt

SEQ ID NO: 64 - DNA, 77 bp, synthetic construct
  source 1..77: mol_type = other DNA
gactggacccctgccggcaggcggcagtgcaggcctgggtgctgccccctagcgggttagggacgtacatccctggg

SEQ ID NO: 65 - DNA, 458 bp, synthetic construct
  source 1..458: mol_type = other DNA
actgtgcgacacagagtatcttttattcatccgtgtaagcaataagcaataacagaacagaacagaatgtttataataataacatttgtgattgtattcgcataggcatattagatcggatgatcgacaacaactacaaaaccggaatggggaagacagagagagagagagagagagagagagaggggggggggaggaggggtggaggaggaggaggagagggggttgcgtctgagttgtagttgcagtaggagtttattatgaattacagagactattatgaattacagagactttattctttcttgtatatttggtggccctgataagggccgttgaggtgttgaggtttgcgagtgcaaaacctttagcttgatttgccggtcttcttgggaagcagcacagcctggatgttgggcagtacaccacctcgagcgatggtcacacctccaagaagc

SEQ ID NO: 66 - DNA, 1214 bp, synthetic construct
  source 1..1214: mol_type = other DNA
agccccatcctcactgactccgtcctggagttggatgagagataatggccttacgttgtgccaggggagggtcgggctggatttagcaagatttaccttctccaaagagcggtgctgcagtggcacagctgcccacggaggtgggggggtcaccgtccctggaggtgatgaagaactgtggggatgtggcactgagggacatggccagtgggcacggtgggtgggttggggttggtcttggggatcttggagggcttttccagccttcatgatttgacgattgtatgaacatctacatggcaattctccagctgcctgtcccagtcctactgacccagctgtatctctccaggcaagctcttccaccccttctgcttgcatccagacaccatcaaacatgcaggctcagacacagggaccagcagtgtctgtggcctttttgtgctcctctccatgctgggttttaacttgctctttgtccttctatcctatcttcttatccttaaggctgttctgaacgctgtgacttggagagtgtcccagagccctcaacacctgcatgtcccacgtccatgctgtcctgcacttccttatccccaagatctgcctctccgtgatgcactgaattggcaaacatgtgtcaccccagaccaacaatgtcacagcaaactcccccttgataggacaagggggaatggctttacactgagacaggggaggtttgggttggatatgaggaggcagtttttcccccagagggtggtgacgcactgaacaggttgcccaaggaggctgtggatgccccatccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctc

SEQ ID NO: 67 - DNA, 260 bp, synthetic construct
  source 1..260: mol_type = other DNA
ggatgctggaggaggtcacctggctcaggaccaggctggtgggggcagggagggtgtccagggctggagccgctgctgctggagtagcgggagtggggggcaggtggtcactgcacgggcagctgtgtccacgctccccctggtggccactcacaggagggacaggaaagctgctccccagcccttctgtggacagatgtaagggggtgctgcagaccccctcagagccacaggagccggcctcaggtaccacacgggag

SEQ ID NO: 68 - DNA, 325 bp, synthetic construct
  source 1..325: mol_type = other DNA
aggatgggctatgaggtggtcgagggaccatgcattctgggtgcacagctgaggaaaagccccagaggatgtccccccgcgttcataccctaagagaagatgcaacaggaacaagaaattgcacaacagagctctgtggaccatgaaaaggccccttcctccacagtgtccctccggcgccctctactgacaaagcttgatatcgtgcttgctgcaaagaagggcttagagtccagtccatcaaggagcaggcactcacggggaagttggaagctgagaggcaagaagaaaccagggctctgcaaactacggccaccattcagac

SEQ ID NO: 69 - DNA, 409 bp, synthetic construct
  source 1..409: mol_type = other DNA
atccctgcaggcattcaaggccaggctggatgtggctctgggcagcctgggctgctggttgatgaccctgcacatagcagggggttggatctggatgagcactgtgctcctttgcaacccaggccgttctatgattctgtcattctaaatctctctttcagcctaaagctttttccccgtatccccccaggtgtctgcaggctcaaagagcagcgagaagcgttcagaggaaagcgatcccgtgccaccttccccgtgcccgggctgtccccgcacgctgccggctcggggatgcggggggagcgccggaccggagcggagccccgggcggctcgctgctgccccctagcgggggagggacgtaattacatccctgggggctttgggggggggctgtccccgtgagctc

SEQ ID NO: 70 - DNA, 409 bp, synthetic construct
  source 1..409: mol_type = other DNA
gagctcacggggacagcccccccccaaagcccccagggatgtaattacgtccctcccccgctagggggcagcagcgagccgcccggggctccgctccggtccggcgctccccccgcatccccgagccggcagcgtgcggggacagcccgggcacggggaaggtggcacgggatcgctttcctctgaacgcttctcgctgctctttgagcctgcagacacctggggggatacggggaaaaagctttaggctgaaagagagatttagaatgacagaatcatagaacggcctgggttgcaaaggagcacagtgctcatccagatccaaccccctgctatgtgcagggtcatcaaccagcagcccaggctgcccagagccacatccagcctggccttgaatgcctgcagggat

(19) United States
(12) Patent Application Publication
(10) Pub. No.: US 2025/0340866 A1
(43) Pub. Date: Nov. 6, 2025

(54) A SCREENING METHOD

(71) Applicant: CSL BEHRING LLC, King of Prussia, PA (US)

(72) Inventors: Florian AESCHIMANN, Melbourne, Victoria (AU); Christian MONTELLESE, Melbourne, Victoria (AU)

(21) Appl. No.: 19/126,458

(22) PCT Filed: Nov. 2, 2023
(86) PCT No.: PCT/IB2023/061019

§ 371 Date: May 1, 2025

Related U.S. Application Data
(60) Provisional application No. 63/381,947, filed on Nov. 2, 2022.

(51) Int. Cl. C12N 15/10

(52) U.S. Cl. CPC C12N 15/1082

(57) ABSTRACT

The present disclosure relates to landing pads, proviruses and methods of use thereof.

### RELATED APPLICATION DATA

The present application claims priority from U.S. Patent Application No. 63/381,947 filed 2 Nov. 2022 entitled “A Screening Method”, the entire contents of which is hereby incorporated by reference.

### SEQUENCE LISTING

The present application is filed together with a Sequence Listing in electronic format. The entire contents of the Sequence Listing are hereby incorporated by reference.

### FIELD

The present disclosure relates to landing pads, proviruses and methods of use thereof.

### BACKGROUND

Retroviruses, e.g., lentiviruses are one of the most studied viral vectors for gene therapy. Retroviruses in general are RNA-based viruses which integrate their genetic information into the target cell chromosomes permanently. The advantages of retroviruses include long-term transgene expression in target cells, a low immunogenic potential, and the ability to transduce into dividing and non-dividing cells.

Lentiviruses are genetically engineered and usually based on human immunodeficiency virus 1 (HIV-1). To increase safety, modern vectors contain only those HIV genes which are necessary for infection and gene delivery, but the genes necessary for replication and virulence factors have been removed.

To produce lentiviruses, cells are transfected with 3-4 plasmids. These include the transfer plasmid with the gene of interest and several packaging plasmids and essential viral proteins responsible for gene integration or self-assembly. These plasmids can be transiently transfected into the cells, or a producer cell line is created with stable integration of the plasmids with inducible promoters, in which lentivirus production can be induced.

Once the virus production has been induced, the release of the virus occurs by budding after successful assembly within the cells. The lentivirus is harvested from the producer cells and subsequently purified and concentrated in the downstream process. The resultant lentivirus can be used to modify patient cells, such as hemopoietic stem cells, for clinical benefit.

The capacity to integrate transgenes into the host cell genome makes retroviral vectors an attractive approach for gene therapy. Clinical trials in patients with X-linked severe combined immunodeficiency disease (X-SCID), adenosine deaminase (ADA)—deficient SCID, chronic granulomatous disease (CGD) and Wiskott-Aldrich syndrome (WAS) have demonstrated clinical benefits and at least temporary functional correction of immune cells. Although stable insertion can provide successful correction, these studies have also highlighted the occurrence of vector-associated insertional mutagenesis or genotoxicity and have raised concerns about long-term safety and efficacy of the use of such vectors for gene therapy.

In addition to the issues of genotoxicity of retroviral integration, production of clinical grade lentiviral vectors are cost intensive, require large amounts of GMP-grade plasmids and hamper process scalability and reproducibility.

Thus, there is a need in the art for improved viral vectors for gene therapy, in particular production of lentiviral vectors with reduced genotoxicity and/or improved safety, whilst preserving therapeutic efficacy.

### SUMMARY

In work leading up to the invention, the inventors sought to produce integrating viral vectors such as lentiviral constructs (i.e., provirus constructs) with reduced genotoxicity, improved safety and/or enhanced efficacy for use in e.g., gene therapy. To this end, the inventors sought to examine genomic loci associated with adverse events or genotoxicity (e.g, within or near the LMO2 gene), especially adverse events or genotoxicity associated with integration of viral vectors. Consequently, the inventors developed landing pad cassettes to facilitate site-specific integration into loci of interest. The loci of interest may be any suitable genomic loci. For example, the loci of interest may include sites associated with adverse events or genotoxicity (e.g, within or near the LMO2 gene). The resultant stable cell lines comprising a landing pad cassette enable evaluation of vector-associated insertional mutagenesis or genotoxicity. Alternatively, the loci of interest may include a safe-harbour locus, a common integration site (CIS) or other desired locus within the genome. The resultant stable cell lines comprising a landing pad cassette enable site-specific integration of a provirus construct and may be applied in therapeutic studies or vector production, including lentiviral vector production.

In addition, the inventors have developed an assay (i.e., a bulk assay) for expansion and selection of cells containing the integrated provirus sequence. Advantageously, the assay developed by the inventors is faster than standard clonal assays, requires less manual input (i.e., individual clones do not require characterisation) and is more accurate. For example, the assay by the inventors measures thousands of clones simultaneously e.g., for high throughput screening of components of a provirus, as well for assessing safety, genotoxicity and/or efficacy of integrated proviruses or modified provirus components.

Based on the foregoing, the present disclosure provides an isolated polynucleotide comprising: a) a nucleotide sequence comprising a promoter operably linked to a nucleotide sequence encoding a detectable marker and/or a nucleotide sequence encoding a selection marker; and b) a nucleotide sequence encoding a suicide marker.

In one example, the polynucleotide comprises, in order from 5′ to 3′: a) a nucleotide sequence comprising a promoter operably linked to a nucleotide sequence encoding a detectable marker and/or a nucleotide sequence encoding a selection marker; and b) a nucleotide sequence encoding a suicide marker.

The present disclosure provides a landing pad cassette comprising a polynucleotide as described herein. In one example, the landing pad cassette comprises: a) a nucleotide sequence comprising a promoter operably linked to a nucleotide sequence encoding a detectable marker and/or a nucleotide sequence encoding a selection marker; b) a nucleotide sequence encoding a suicide marker; and c) a first site-specific recombination site and a second site-specific recombination site.

In one example, the landing pad cassette comprises, in order from 5′ to 3′: a) a nucleotide sequence comprising a first site-specific recombination site; b) a nucleotide sequence comprising a promoter operably linked to a nucleotide sequence encoding a detectable marker and/or a nucleotide sequence encoding a selection marker; c) a nucleotide sequence encoding a suicide marker; and d) a nucleotide sequence comprising a second site-specific recombination site.

In one example, the landing pad cassette comprises: a) a nucleotide sequence comprising a promoter operably linked to a nucleotide sequence encoding a detectable marker; b) a nucleotide sequence encoding a suicide marker; and c) a first site-specific recombination site and a second site-specific recombination site.

In one example, the landing pad cassette comprises, in order from 5′ to 3′: a) a nucleotide sequence comprising a first site-specific recombination site; b) a nucleotide sequence comprising a promoter operably linked to a nucleotide sequence encoding a detectable marker; c) a nucleotide sequence encoding a suicide marker; and d) a nucleotide sequence comprising a second site-specific recombination site.

In one example, the landing pad cassette comprises: a) a nucleotide sequence comprising a promoter operably linked to a nucleotide sequence encoding a selection marker; b) a nucleotide sequence encoding a suicide marker; and c) a first site-specific recombination site and a second site-specific recombination site.

In one example, the landing pad cassette comprises, in order from 5′ to 3′: a) a nucleotide sequence comprising a first site-specific recombination site; b) a nucleotide sequence comprising a promoter operably linked to a nucleotide sequence encoding a selection marker; c) a nucleotide sequence encoding a suicide marker; and d) a nucleotide sequence comprising a second site-specific recombination site.

In one example, the landing pad cassette comprises: a) a nucleotide sequence comprising a promoter operably linked to a nucleotide sequence encoding a detectable marker and a nucleotide sequence encoding a selection marker; b) a nucleotide sequence encoding a suicide marker; and c) a first site-specific recombination site and a second site-specific recombination site.

In one example, the landing pad cassette comprises, in order from 5′ to 3′: a) a nucleotide sequence comprising a first site-specific recombination site; b) a nucleotide sequence comprising a promoter operably linked to a nucleotide sequence encoding a detectable marker and a nucleotide sequence encoding a selection marker; c) a nucleotide sequence encoding a suicide marker; and d) a nucleotide sequence comprising a second site-specific recombination site.

In one example, the site-specific recombination sites are selected from the group consisting of attP recombination sites and loxP recombination sites.

In one example, the site-specific recombination sites are attP recombination sites. For example, the first site-specific recombination site is an attP (GT) recombination site and the second site-specific recombination site an attP (GA) recombination site.

In one example, the site-specific recombination sites are loxP recombination sites.

In one example, the promoter is a cytomegalovirus (CMV) promoter, a CMV enhancer, a murine leukemia virus-derived (MND) promoter, a simian virus 40 (SV40) promoter with enhancer, a polyubiquitin C gene (UBC) promoter, a phosphoglycerate kinase (PGK) promoter, an elongation factor-1 alpha (EF1A) promoter, a human β-actin (hACTB) promoter, a 7SK promoter or a cytomegalovirus immediate-early enhancer/chicken β-actin (CAG) promoter. For example, the promoter is a CMV promoter. In one example, the promoter is a CMV enhancer. In one example, the promoter is a MND promoter. In one example, the promoter is a SV40 promoter with enhancer. In one example, the promoter is an UBC promoter. In one example, the promoter is a PGK promoter. In one example, the promoter is an EFIA promoter. In one example, the promoter is a hACTB promoter. In one example, the promoter is a CAG promoter.

In one example, the nucleotide sequence comprising the promoter comprises or consists of a sequence set forth in any one of SEQ ID NOs: 3 to 5. For example, the nucleotide sequence comprising the promoter comprises or consists of a sequence set forth in SEQ ID NO: 3. In another example, the nucleotide sequence comprising the promoter comprises or consists of a sequence set forth in SEQ ID NO: 4. In a further example, the nucleotide sequence comprising the promoter comprises or consists of a sequence set forth in SEQ ID NO: 5.

In one example, the nucleotide sequence comprising the CMV promoter comprises or consists of a sequence set forth in SEQ ID NO: 3. In one example, the nucleotide sequence comprising the CMV promoter comprises of a sequence set forth in SEQ ID NO: 3. In one example, the nucleotide sequence comprising the CMV promoter consists of a sequence set forth in SEQ ID NO: 3.

In one example, the nucleotide sequence comprising the CMV promoter comprises or consists of a sequence set forth in SEQ ID NO: 4. In one example, the nucleotide sequence comprising the CMV promoter comprises of a sequence set forth in SEQ ID NO: 4. In one example, the nucleotide sequence comprising the CMV promoter consists of a sequence set forth in SEQ ID NO: 4.

In one example, the nucleotide sequence comprising the MND promoter comprises or consists of a sequence set forth in SEQ ID NO: 5. In one example, the nucleotide sequence comprising the MND promoter comprises of a sequence set forth in SEQ ID NO: 5. In one example, the nucleotide sequence comprising the MND promoter consists of a sequence set forth in SEQ ID NO: 5.

In one example, the detectable marker is an enhanced green fluorescent protein (cGFP), a red fluorescent protein (mCherry or mScarlet), a yellow fluorescent protein or a cyan fluorescent protein. For example, the detectable marker is an eGFP. In one example, the detectable marker is a mCherry. In one example, the detectable marker is an mScarlet. In one example, the detectable marker is a yellow fluorescent protein. In one example, the detectable marker is a cyan fluorescent protein.

In one example, the nucleotide sequence encoding the eGFP comprises or consists of a sequence set forth in SEQ ID NO: 7. In one example, the nucleotide sequence encoding the cGFP comprises of a sequence set forth in SEQ ID NO: 7. In one example, the nucleotide sequence encoding the eGFP consists of a sequence set forth in SEQ ID NO: 7.

In one example, the suicide marker is Herpes Simplex Virus-1 thymidine kinase (HSV-TK), thymidine kinase (TK), caspase-9, caspase-8, purine nucleoside phosphorylase, uracil phosphoribosyl transferase or cytosine deaminase. For example, the suicide marker is HSV-TK. In one example, the suicide marker is thymidine kinase (TK). In one example, the suicide marker is caspase-9. In one example, the suicide marker is caspase-8. In one example, the suicide marker is purine nucleoside phosphorylase. In one example, the suicide marker is uracil phosphoribosyl transferase. In one example, the suicide marker is cytosine deaminase. In one example, the nucleotide sequence encoding HSV-TK comprises or consists of a sequence set forth in SEQ ID NO: 6. In one example, the nucleotide sequence encoding HSV-TK comprises of a sequence set forth in SEQ ID NO: 6. In one example, the nucleotide sequence encoding HSV-TK consists of a sequence set forth in SEQ ID NO: 6.

In one example, the selection marker is a neomycin resistance gene, a hygromycin resistance gene, a puromycin N-acetyl-transferase, a histidinol dehyrogenase, a zeocin resistance gene, a bleomycin resistance gene or a blasticidin S deaminase. For example, the selection marker is a puromycin N-acetyl-transferase. In one example, the selection marker is a neomycin. In one example, the selection marker is a hygromycin. In one example, the selection marker is a histidinol dehyrogenase. In one example, the selection marker is a zeocin. In one example, the selection marker is a blasticidin S deaminase.

In one example, the nucleotide sequence encoding the puromycin N-acetyl-transferase comprises or consists of a sequence set forth in SEQ ID NO: 8. In one example, the nucleotide sequence encoding the puromycin N-acetyl-transferase comprises of a sequence set forth in SEQ ID NO: 8. In one example, the nucleotide sequence encoding the puromycin N-acetyl-transferase consists of a sequence set forth in SEQ ID NO: 8.

In one example, the landing pad cassette comprises: a) a first attP (GT) recombination site and a second attP (GA) recombination site; b) a nucleotide sequence encoding a CMV promoter comprising a sequence set forth in SEQ ID NO: 3 or 4, or a nucleotide sequence encoding a MND promoter comprising a sequence set forth in SEQ ID NO: 5; c) a nucleotide sequence encoding an eGFP comprising a sequence set forth in SEQ ID NO: 7; and d) a nucleotide sequence encoding a HSV-TK comprising a sequence set forth in SEQ ID NO: 6; and/or a nucleotide sequence encoding a puromycin N-acetyl-transferase comprising a sequence set forth in SEQ ID NO: 8.

In one example, the landing pad cassette comprises: a) a first attP (GT) recombination site and a second attP (GA) recombination site; b) a nucleotide sequence encoding a CMV promoter comprising a sequence set forth in SEQ ID NO: 3; c) a nucleotide sequence encoding an eGFP comprising a sequence set forth in SEQ ID NO: 7; and d) a nucleotide sequence encoding a HSV-TK comprising a sequence set forth in SEQ ID NO: 6.

In one example, the landing pad cassette comprises, in order from 5′ to 3′: a) a first attP (GT) recombination site: b) a nucleotide sequence encoding a CMV promoter comprising a sequence set forth in SEQ ID NO: 3: c) a nucleotide sequence encoding an eGFP comprising a sequence set forth in SEQ ID NO: 7; d) a nucleotide sequence encoding a HSV-TK comprising a sequence set forth in SEQ ID NO: 6; and e) a second attP (GA) recombination site.

In one example, the landing pad cassette comprises: a) a first attP (GT) recombination site and a second attP (GA) recombination site; b) a nucleotide sequence encoding a CMV promoter comprising a sequence set forth in SEQ ID NO: 4; c) a nucleotide sequence encoding an eGFP comprising a sequence set forth in SEQ ID NO: 7; and d) a nucleotide sequence encoding a HSV-TK comprising a sequence set forth in SEQ ID NO: 6.

In one example, the landing pad cassette comprises, in order from 5′ to 3′: a) a first attP (GT) recombination site: b) a nucleotide sequence encoding a CMV promoter comprising a sequence set forth in SEQ ID NO: 4: c) a nucleotide sequence encoding an eGFP comprising a sequence set forth in SEQ ID NO: 7; d) a nucleotide sequence encoding a HSV-TK comprising a sequence set forth in SEQ ID NO: 6; and e) a second attP (GA) recombination site. In one example, the landing pad cassette comprises: a) a first attP (GT) recombination site and a second attP (GA) recombination site; b) a nucleotide sequence encoding a MND promoter comprising a sequence set forth in SEQ ID NO: 5; c) a nucleotide sequence encoding an cGFP comprising a sequence set forth in SEQ ID NO: 7; and d) a nucleotide sequence encoding a HSV-TK comprising a sequence set forth in SEQ ID NO: 6; and/or a nucleotide sequence encoding a puromycin N-acetyl-transferase comprising a sequence set forth in SEQ ID NO: 8.

In one example, the landing pad cassette comprises, in order from 5′ to 3′: a) a first attP (GT) recombination site; b) a nucleotide sequence encoding a MND promoter comprising a sequence set forth in SEQ ID NO: 5: c) a nucleotide sequence encoding an cGFP comprising a sequence set forth in SEQ ID NO: 7; d) a nucleotide sequence encoding a HSV-TK comprising a sequence set forth in SEQ ID NO: 6; and/or a nucleotide sequence encoding a puromycin N-acetyl-transferase comprising a sequence set forth in SEQ ID NO: 8; and e) a second attP (GA) recombination site.

In one example, the landing pad cassette comprises a nucleotide sequence comprising a linker located between the detectable marker and the suicide marker.

In one example, the nucleotide sequence comprising the linker is located between the 3′ end of the nucleotide sequence encoding the detectable marker and the 5′ end of the nucleotide sequence comprising the suicide marker.

In one example, the landing pad cassette comprises a nucleotide sequence comprising a linker located between the selection marker and the suicide marker.

In one example, the nucleotide sequence comprising the linker is located between the 3′ end of the nucleotide sequence encoding the selection marker and the 5′ end of the nucleotide sequence comprising the suicide marker.

In one example, the nucleotide sequence comprising the linker is an internal ribosome entry site (IRES) or encodes a self-cleaving peptide. For example, the self-cleaving peptide is a 2A self-cleaving peptide. In one example, the 2A self-cleaving peptide is selected from the group consisting of a P2A. T2A, E2A or a F2A self-cleaving peptide. In one example, the nucleotide sequence comprising the linker is an internal ribosome entry site (IRES). In one example, the nucleotide sequence comprising the linker encodes a self-cleaving peptide. For example, the nucleotide sequence comprising the linker encodes a 2A self-cleaving peptide.

For example, the 2A self-cleaving peptide is selected from the group consisting of a P2A, T2A, E2A or a F2A self-cleaving peptide. In one example, the 2A self-cleaving peptide is a P2A. In one example, the 2A self-cleaving peptide is a T2A. In one example, the 2A self-cleaving peptide is an E2A. In one example, the 2A self-cleaving peptide is a F2A.

In one example, the nucleotide sequence encoding the P2A self-cleaving peptide comprises or consists of a sequence set forth in any one of SEQ ID NOs: 10-13. In one example, the nucleotide sequence encoding the P2A self-cleaving peptide comprises of a sequence set forth in any one of SEQ ID NOs: 10-13. In one example, the nucleotide sequence encoding the P2A self-cleaving peptide consists of a sequence set forth in any one of SEQ ID NOs: 10-13.

In one example, the nucleotide sequence encoding the P2A self-cleaving peptide comprises or consists of a sequence set forth in SEQ ID NO: 10. In one example, the nucleotide sequence encoding the P2A comprises of a sequence set forth in SEQ ID NO: 10. In one example, the nucleotide sequence encoding the P2A consists of a sequence set forth in SEQ ID NO: 10.

In one example, the nucleotide sequence encoding the P2A self-cleaving peptide comprises or consists of a sequence set forth in SEQ ID NO: 11. In one example, the nucleotide sequence encoding the P2A comprises of a sequence set forth in SEQ ID NO: 11. In one example, the nucleotide sequence encoding the P2A consists of a sequence set forth in SEQ ID NO: 11.

In one example, the nucleotide sequence encoding the P2A self-cleaving peptide comprises or consists of a sequence set forth in SEQ ID NO: 12. In one example, the nucleotide sequence encoding the P2A comprises of a sequence set forth in SEQ ID NO: 12. In one example, the nucleotide sequence encoding the P2A consists of a sequence set forth in SEQ ID NO: 12.

In one example, the nucleotide sequence encoding the P2A self-cleaving peptide comprises or consists of a sequence set forth in SEQ ID NO: 13. In one example, the nucleotide sequence encoding the P2A comprises of a sequence set forth in SEQ ID NO: 13. In one example, the nucleotide sequence encoding the P2A consists of a sequence set forth in SEQ ID NO: 13.

In one example, the nucleotide sequence encoding the T2A self-cleaving peptide comprises or consists of a sequence set forth in SEQ ID NO: 15 or 16. In one example, the nucleotide sequence encoding the T2A comprises of a sequence set forth in SEQ ID NO: 15 or 16. In one example, the nucleotide sequence encoding the T2A consists of a sequence set forth in SEQ ID NO: 15 or 16.

In one example, the nucleotide sequence encoding the T2A self-cleaving peptide comprises or consists of a sequence set forth in SEQ ID NO: 15. In one example, the nucleotide sequence encoding the T2A comprises of a sequence set forth in SEQ ID NO: 15. In one example, the nucleotide sequence encoding the T2A consists of a sequence set forth in SEQ ID NO: 15.

In one example, the nucleotide sequence encoding the T2A self-cleaving peptide comprises or consists of a sequence set forth in SEQ ID NO: 16. In one example, the nucleotide sequence encoding the T2A comprises of a sequence set forth in SEQ ID NO: 16. In one example, the nucleotide sequence encoding the T2A consists of a sequence set forth in SEQ ID NO: 16.

In one example, the landing pad cassette comprises a nucleotide sequence comprising a poly A signal located 3′ of the suicide marker. For example, the poly A signal is a simian virus 40 (SV40) polyA, simian virus 40 late (SVLP) polyA, human growth hormone (hGH) poly A, bovine growth hormone (BGH) polyA or rabbit beta-globin (rbGlob) polyA. In one example, the polyA signal is a SV40 polyA. In one example, the polyA signal is a SVLP polyA. In one example, the polyA signal is a hGH polyA. In one example, the poly A signal is a BGH polyA. In one example, the polyA signal is a rbGlob polyA.

In one example, the nucleotide sequence comprising the polyA signal comprises or consists of a sequence set forth in SEQ ID NO: 17 or SEQ ID NO: 55. In one example, the nucleotide sequence comprising the poly A signal comprises or consists of a sequence set forth in SEQ ID NO: 17. In another example, the nucleotide sequence comprising the poly A signal comprises or consists of a sequence set forth in SEQ ID NO: 55. In one example, the nucleotide sequence comprising the polyA signal comprises of a sequence set forth in SEQ ID NO: 17 or SEQ ID NO: 55. In one example, the nucleotide sequence comprising the polyA signal comprises of a sequence set forth in SEQ ID NO: 17. In another example, the nucleotide sequence comprising the poly A signal comprises of a sequence set forth in SEQ ID NO: 55. In one example, the nucleotide sequence comprising the polyA signal consists of a sequence set forth in SEQ ID NO: 17 or SEQ ID NO: 55. In one example, the nucleotide sequence comprising the poly A signal consists of a sequence set forth in SEQ ID NO: 17. In one example, the nucleotide sequence comprising the polyA signal consists of a sequence set forth in SEQ ID NO: 55.

In one example, the landing pad cassette comprises a nucleotide sequence comprising a woodchuck hepatitis virus post-transcriptional regulatory element (WPRE) located 3′ of the suicide marker. In one example, the landing pad cassette comprises a nucleotide sequence comprising a woodchuck hepatitis virus post-transcriptional regulatory element (WPRE) located 3′ of the suicide marker and 5′ of the poly A signal. For example, the WPRE comprises or consists of a sequence set forth in SEQ ID NO: 20. In one example, the nucleotide sequence comprising the WPRE comprises of a sequence set forth in SEQ ID NO: 20. In one example, the nucleotide sequence comprising the WPRE consists of a sequence set forth in SEQ ID NO: 20.

For example, the landing pad cassette comprises, in order from 5′ to 3′: a) a nucleotide sequence comprising an attP (GT) recombination site: b) a nucleotide sequence comprising a CMV promoter; c) a nucleotide sequence encoding an enhanced green fluorescent protein (cGFP) detectable marker; d) a nucleotide sequence encoding a P2A self-cleaving peptide linker; e) a nucleotide sequence encoding a Herpes Simplex Virus-1 thymidine kinase (HSV-TK) suicide marker; f) a nucleotide sequence comprising a WPRE; g) a nucleotide sequence comprising a BGH poly A signal; and h) a nucleotide sequence comprising an attP (GA) site.

In one example, the landing pad cassette comprises, in order from 5′ to 3′: a) a nucleotide sequence comprising an attP (GT) recombination site; b) a nucleotide sequence comprising a CMV promoter comprising a sequence set forth in SEQ ID NO: 3; c) a nucleotide sequence encoding an enhanced green fluorescent protein (eGFP) detectable marker comprising a sequence set forth in SEQ ID NO: 7; d) a nucleotide sequence encoding a P2A self-cleaving peptide linker comprising a sequence set forth in SEQ ID NO: 10; e) a nucleotide sequence encoding a Herpes Simplex Virus-1 thymidine kinase (HSV-TK) suicide marker comprising a sequence set forth in SEQ ID NO: 6; f) a nucleotide sequence comprising a WPRE comprising a sequence set forth in SEQ ID NO: 20; g) a nucleotide sequence comprising a BGH polyA signal comprising a sequence set forth in SEQ ID NO: 55; and h) a nucleotide sequence comprising an attP (GA) site.

In one example, the landing pad cassette comprises, in order from 5′ to 3′: a) a nucleotide sequence comprising an attP (GT) recombination site; b) a nucleotide sequence comprising a CMV promoter comprising a sequence set forth in SEQ ID NO: 4; c) a nucleotide sequence encoding an enhanced green fluorescent protein (eGFP) detectable marker comprising a sequence set forth in SEQ ID NO: 7; d) a nucleotide sequence encoding a P2A self-cleaving peptide linker comprising a sequence set forth in SEQ ID NO: 10; e) a nucleotide sequence encoding a Herpes Simplex Virus-1 thymidine kinase (HSV-TK) suicide marker comprising a sequence set forth in SEQ ID NO: 6; f) a nucleotide sequence comprising a WPRE comprising a sequence set forth in SEQ ID NO: 20; g) a nucleotide sequence comprising a BGH polyA signal comprising a sequence set forth in SEQ ID NO: 55; and h) a nucleotide sequence comprising an attP (GA) site.

In one example, the landing pad cassette comprises, in order from 5′ to 3′: a) a nucleotide sequence comprising an attP (GT) recombination site; b) a nucleotide sequence comprising a CMV promoter comprising a sequence set forth in SEQ ID NO: 3; c) a nucleotide sequence encoding an enhanced green fluorescent protein (eGFP) detectable marker comprising a sequence set forth in SEQ ID NO: 7; d) a nucleotide sequence encoding a P2A self-cleaving peptide linker comprising a sequence set forth in SEQ ID NO: 11; e) a nucleotide sequence encoding a Herpes Simplex Virus-1 thymidine kinase (HSV-TK) suicide marker comprising a sequence set forth in SEQ ID NO: 6; f) a nucleotide sequence comprising a WPRE comprising a sequence set forth in SEQ ID NO: 20; g) a nucleotide sequence comprising a BGH polyA signal comprising a sequence set forth in SEQ ID NO: 55; and h) a nucleotide sequence comprising an attP (GA) site.

In one example, the landing pad cassette comprises, in order from 5′ to 3′: a) a nucleotide sequence comprising an attP (GT) recombination site; b) a nucleotide sequence comprising a CMV promoter comprising a sequence set forth in SEQ ID NO: 4; c) a nucleotide sequence encoding an enhanced green fluorescent protein (eGFP) detectable marker comprising a sequence set forth in SEQ ID NO: 7; d) a nucleotide sequence encoding a P2A self-cleaving peptide linker comprising a sequence set forth in SEQ ID NO: 11; e) a nucleotide sequence encoding a Herpes Simplex Virus-1 thymidine kinase (HSV-TK) suicide marker comprising a sequence set forth in SEQ ID NO: 6; f) a nucleotide sequence comprising a WPRE comprising a sequence set forth in SEQ ID NO: 20; g) a nucleotide sequence comprising a BGH polyA signal comprising a sequence set forth in SEQ ID NO: 55; and h) a nucleotide sequence comprising an attP (GA) site.

In one example, the landing pad cassette comprises, in order from 5′ to 3′: a) a nucleotide sequence comprising an attP (GT) recombination site; b) a nucleotide sequence comprising a CMV promoter comprising a sequence set forth in SEQ ID NO: 3; c) a nucleotide sequence encoding an enhanced green fluorescent protein (eGFP) detectable marker comprising a sequence set forth in SEQ ID NO: 7; d) a nucleotide sequence encoding a P2A self-cleaving peptide linker comprising a sequence set forth in SEQ ID NO: 12; e) a nucleotide sequence encoding a Herpes Simplex Virus-1 thymidine kinase (HSV-TK) suicide marker comprising a sequence set forth in SEQ ID NO: 6; f) a nucleotide sequence comprising a WPRE comprising a sequence set forth in SEQ ID NO: 20; g) a nucleotide sequence comprising a BGH polyA signal comprising a sequence set forth in SEQ ID NO: 55; and h) a nucleotide sequence comprising an attP (GA) site.

In one example, the landing pad cassette comprises, in order from 5′ to 3′: a) a nucleotide sequence comprising an attP (GT) recombination site: b) a nucleotide sequence comprising a CMV promoter comprising a sequence set forth in SEQ ID NO: 4; c) a nucleotide sequence encoding an enhanced green fluorescent protein (eGFP) detectable marker comprising a sequence set forth in SEQ ID NO: 7; d) a nucleotide sequence encoding a P2A self-cleaving peptide linker comprising a sequence set forth in SEQ ID NO: 12; e) a nucleotide sequence encoding a Herpes Simplex Virus-1 thymidine kinase (HSV-TK) suicide marker comprising a sequence set forth in SEQ ID NO: 6; f) a nucleotide sequence comprising a WPRE comprising a sequence set forth in SEQ ID NO: 20; g) a nucleotide sequence comprising a BGH polyA signal comprising a sequence set forth in SEQ ID NO: 55; and h) a nucleotide sequence comprising an attP (GA) site.

In one example, the landing pad cassette comprises, in order from 5′ to 3′: a) a nucleotide sequence comprising an attP (GT) recombination site; b) a nucleotide sequence comprising a CMV promoter comprising a sequence set forth in SEQ ID NO: 3; c) a nucleotide sequence encoding an enhanced green fluorescent protein (eGFP) detectable marker comprising a sequence set forth in SEQ ID NO: 7; d) a nucleotide sequence encoding a P2A self-cleaving peptide linker comprising a sequence set forth in SEQ ID NO: 13; e) a nucleotide sequence encoding a Herpes Simplex Virus-1 thymidine kinase (HSV-TK) suicide marker comprising a sequence set forth in SEQ ID NO: 6; 1) a nucleotide sequence comprising a WPRE comprising a sequence set forth in SEQ ID NO: 20; g) a nucleotide sequence comprising a BGH polyA signal comprising a sequence set forth in SEQ ID NO: 55; and h) a nucleotide sequence comprising an attP (GA) site.

In one example, the landing pad cassette comprises, in order from 5′ to 3′: a) a nucleotide sequence comprising an attP (GT) recombination site; b) a nucleotide sequence comprising a CMV promoter comprising a sequence set forth in SEQ ID NO: 4; c) a nucleotide sequence encoding an enhanced green fluorescent protein (eGFP) detectable marker comprising a sequence set forth in SEQ ID NO: 7; d) a nucleotide sequence encoding a P2A self-cleaving peptide linker comprising a sequence set forth in SEQ ID NO: 13; e) a nucleotide sequence encoding a Herpes Simplex Virus-1 thymidine kinase (HSV-TK) suicide marker comprising a sequence set forth in SEQ ID NO: 6; f) a nucleotide sequence comprising a WPRE comprising a sequence set forth in SEQ ID NO: 20; g) a nucleotide sequence comprising a BGH polyA signal comprising a sequence set forth in SEQ ID NO: 55; and h) a nucleotide sequence comprising an attP (GA) site.

In one example, the landing pad cassette comprises, in order from 5′ to 3′: a) a nucleotide sequence comprising an attP (GT) recombination site: b) a nucleotide sequence comprising a CMV promoter comprising a sequence set forth in SEQ ID NO: 3; c) a nucleotide sequence encoding an enhanced green fluorescent protein (eGFP) detectable marker comprising a sequence set forth in SEQ ID NO: 7; d) a nucleotide sequence encoding a T2A self-cleaving peptide linker comprising a sequence set forth in SEQ ID NO: 15; e) a nucleotide sequence encoding a Herpes Simplex Virus-1 thymidine kinase (HSV-TK) suicide marker comprising a sequence set forth in SEQ ID NO: 6; f) a nucleotide sequence comprising a WPRE comprising a sequence set forth in SEQ ID NO: 20; g) a nucleotide sequence comprising a BGH polyA signal comprising a sequence set forth in SEQ ID NO: 55; and h) a nucleotide sequence comprising an attP (GA) site.

In one example, the landing pad cassette comprises, in order from 5′ to 3′: a) a nucleotide sequence comprising an attP (GT) recombination site; b) a nucleotide sequence comprising a CMV promoter comprising a sequence set forth in SEQ ID NO: 3; c) a nucleotide sequence encoding an enhanced green fluorescent protein (eGFP) detectable marker comprising a sequence set forth in SEQ ID NO: 7; d) a nucleotide sequence encoding a T2A self-cleaving peptide linker comprising a sequence set forth in SEQ ID NO: 16; e) a nucleotide sequence encoding a Herpes Simplex Virus-1 thymidine kinase (HSV-TK) suicide marker comprising a sequence set forth in SEQ ID NO: 6; f) a nucleotide sequence comprising a WPRE comprising a sequence set forth in SEQ ID NO: 20; g) a nucleotide sequence comprising a BGH polyA signal comprising a sequence set forth in SEQ ID NO: 55; and h) a nucleotide sequence comprising an attP (GA) site.

In one example, the landing pad cassette comprises, in order from 5′ to 3′: a) a nucleotide sequence comprising an attP (GT) recombination site; b) a nucleotide sequence comprising a CMV promoter comprising a sequence set forth in SEQ ID NO: 4; c) a nucleotide sequence encoding an enhanced green fluorescent protein (eGFP) detectable marker comprising a sequence set forth in SEQ ID NO: 7; d) a nucleotide sequence encoding a T2A self-cleaving peptide linker comprising a sequence set forth in SEQ ID NO: 15; e) a nucleotide sequence encoding a Herpes Simplex Virus-1 thymidine kinase (HSV-TK) suicide marker comprising a sequence set forth in SEQ ID NO: 6; f) a nucleotide sequence comprising a WPRE comprising a sequence set forth in SEQ ID NO: 20; g) a nucleotide sequence comprising a BGH polyA signal comprising a sequence set forth in SEQ ID NO: 55; and h) a nucleotide sequence comprising an attP (GA) site.

In one example, the landing pad cassette comprises, in order from 5′ to 3′: a) a nucleotide sequence comprising an attP (GT) recombination site; b) a nucleotide sequence comprising a CMV promoter comprising a sequence set forth in SEQ ID NO: 4; c) a nucleotide sequence encoding an enhanced green fluorescent protein (eGFP) detectable marker comprising a sequence set forth in SEQ ID NO: 7; d) a nucleotide sequence encoding a T2A self-cleaving peptide linker comprising a sequence set forth in SEQ ID NO: 16; e) a nucleotide sequence encoding a Herpes Simplex Virus-1 thymidine kinase (HSV-TK) suicide marker comprising a sequence set forth in SEQ ID NO: 6; f) a nucleotide sequence comprising a WPRE comprising a sequence set forth in SEQ ID NO: 20; g) a nucleotide sequence comprising a BGH polyA signal comprising a sequence set forth in SEQ ID NO: 55; and h) a nucleotide sequence comprising an attP (GA) site.

In one example, the landing pad cassette comprises, in order from 5′ to 3′: a) a nucleotide sequence comprising an attP (GT) recombination site; b) a nucleotide sequence comprising a MND promoter comprising a sequence set forth in SEQ ID NO: 5; c) a nucleotide sequence encoding an enhanced green fluorescent protein (eGFP) detectable marker comprising a sequence set forth in SEQ ID NO: 7; d) a nucleotide sequence encoding a P2A self-cleaving peptide linker comprising a sequence set forth in SEQ ID NO: 10; e) a nucleotide sequence encoding a Herpes Simplex Virus-1 thymidine kinase (HSV-TK) suicide marker comprising a sequence set forth in SEQ ID NO: 6; f) a nucleotide sequence comprising a WPRE comprising a sequence set forth in SEQ ID NO: 20; g) a nucleotide sequence comprising a BGH polyA signal comprising a sequence set forth in SEQ ID NO: 55; and h) a nucleotide sequence comprising an attP (GA) site.

In one example, the landing pad cassette comprises, in order from 5′ to 3′: a) a nucleotide sequence comprising an attP (GT) recombination site: b) a nucleotide sequence comprising a MND promoter comprising a sequence set forth in SEQ ID NO: 5; c) a nucleotide sequence encoding an enhanced green fluorescent protein (eGFP) detectable marker comprising a sequence set forth in SEQ ID NO: 7; d) a nucleotide sequence encoding a P2A self-cleaving peptide linker comprising a sequence set forth in SEQ ID NO: 11; e) a nucleotide sequence encoding a Herpes Simplex Virus-1 thymidine kinase (HSV-TK) suicide marker comprising a sequence set forth in SEQ ID NO: 6; f) a nucleotide sequence comprising a WPRE comprising a sequence set forth in SEQ ID NO: 20; g) a nucleotide sequence comprising a BGH polyA signal comprising a sequence set forth in SEQ ID NO: 55; and h) a nucleotide sequence comprising an attP (GA) site.

In one example, the landing pad cassette comprises, in order from 5′ to 3′: a) a nucleotide sequence comprising an attP (GT) recombination site; b) a nucleotide sequence comprising a MND promoter comprising a sequence set forth in SEQ ID NO: 5; c) a nucleotide sequence encoding an enhanced green fluorescent protein (eGFP) detectable marker comprising a sequence set forth in SEQ ID NO: 7; d) a nucleotide sequence encoding a P2A self-cleaving peptide linker comprising a sequence set forth in SEQ ID NO: 12; c) a nucleotide sequence encoding a Herpes Simplex Virus-1 thymidine kinase (HSV-TK) suicide marker comprising a sequence set forth in SEQ ID NO: 6; f) a nucleotide sequence comprising a WPRE comprising a sequence set forth in SEQ ID NO: 20; g) a nucleotide sequence comprising a BGH polyA signal comprising a sequence set forth in SEQ ID NO: 55; and h) a nucleotide sequence comprising an attP (GA) site.

In one example, the landing pad cassette comprises, in order from 5′ to 3′: a) a nucleotide sequence comprising an attP (GT) recombination site; b) a nucleotide sequence comprising a MND promoter comprising a sequence set forth in SEQ ID NO: 5; c) a nucleotide sequence encoding an enhanced green fluorescent protein (eGFP) detectable marker comprising a sequence set forth in SEQ ID NO: 7; d) a nucleotide sequence encoding a P2A self-cleaving peptide linker comprising a sequence set forth in SEQ ID NO: 13; e) a nucleotide sequence encoding a Herpes Simplex Virus-1 thymidine kinase (HSV-TK) suicide marker comprising a sequence set forth in SEQ ID NO: 6; f) a nucleotide sequence comprising a WPRE comprising a sequence set forth in SEQ ID NO: 20; g) a nucleotide sequence comprising a BGH polyA signal comprising a sequence set forth in SEQ ID NO: 55; and h) a nucleotide sequence comprising an attP (GA) site.

In one example, the landing pad cassette comprises, in order from 5′ to 3′: a) a nucleotide sequence comprising an attP (GT) recombination site: b) a nucleotide sequence comprising a MND promoter comprising a sequence set forth in SEQ ID NO: 5; c) a nucleotide sequence encoding an enhanced green fluorescent protein (eGFP) detectable marker comprising a sequence set forth in SEQ ID NO: 7; d) a nucleotide sequence encoding a T2A self-cleaving peptide linker comprising a sequence set forth in SEQ ID NO: 15; e) a nucleotide sequence encoding a Herpes Simplex Virus-1 thymidine kinase (HSV-TK) suicide marker comprising a sequence set forth in SEQ ID NO: 6; f) a nucleotide sequence comprising a WPRE comprising a sequence set forth in SEQ ID NO: 20; g) a nucleotide sequence comprising a BGH polyA signal comprising a sequence set forth in SEQ ID NO: 55; and h) a nucleotide sequence comprising an attP (GA) site.

In one example, the landing pad cassette comprises, in order from 5′ to 3′: a) a nucleotide sequence comprising an attP (GT) recombination site; b) a nucleotide sequence comprising a MND promoter comprising a sequence set forth in SEQ ID NO: 5; c) a nucleotide sequence encoding an enhanced green fluorescent protein (eGFP) detectable marker comprising a sequence set forth in SEQ ID NO: 7; d) a nucleotide sequence encoding a T2A self-cleaving peptide linker comprising a sequence set forth in SEQ ID NO: 16; c) a nucleotide sequence encoding a Herpes Simplex Virus-1 thymidine kinase (HSV-TK) suicide marker comprising a sequence set forth in SEQ ID NO: 6; f) a nucleotide sequence comprising a WPRE comprising a sequence set forth in SEQ ID NO: 20; g) a nucleotide sequence comprising a BGH polyA signal comprising a sequence set forth in SEQ ID NO: 55; and h) a nucleotide sequence comprising an attP (GA) site.

The present disclosure provides a landing pad plasmid comprising the landing pad cassette as described herein. For example, the landing pad plasmid comprises the landing pad cassette as described herein, and a nucleotide sequence comprising a 5′ homology arm (HA); and a nucleotide sequence comprising a 3′ HA.

In one example, the landing pad plasmid comprises a nucleotide sequence comprising one or more additional promoters. In one example, the one or more additional promoters are not located within the landing pad cassette. For example, the one or more additional promoters are located downstream (i.e., 3′) of the 3′ HA of the landing pad cassette. In one example, the one or more additional promoters is selected from the group consisting of a CMV promoter, a CMV enhancer, a MND promoter, a SV40 promoter with enhancer, a UBC promoter, a PGK promoter, an EF1A promoter, a hACTB promoter, a CAG promoter and combinations thereof. For example, the additional promoter is a CMV promoter. In one example, the additional promoter is a CMV enhancer. In one example, the additional promoter is a MND promoter. In one example, the additional promoter is a SV40 promoter with enhancer. In one example, the additional promoter is a UBC promoter. In one example, the additional promoter is a PGK promoter. In one example, the additional promoter is an EFIA promoter. In one example, the additional promoter is a hACTB promoter. In one example, the additional promoter is a CAG promoter.

In one example, the landing plasmid comprises a nucleotide sequence comprising the SV40 promoter with enhancer comprising or consisting of a sequence set forth in SEQ ID NO: 17. In one example, the nucleotide sequence comprising the SV40 promoter with enhancer comprises of a sequence set forth in SEQ ID NO: 17. In one example, the nucleotide sequence comprising the SV40 promoter with enhancer consists of a sequence set forth in SEQ ID NO: 17.

In one example, the landing plasmid comprises a nucleotide sequence comprising the BGH polyA signal comprising or consisting of a sequence set forth in SEQ ID NO: 55. In one example, the nucleotide sequence comprising the BGH polyA signal comprises of a sequence set forth in SEQ ID NO: 55. In one example, the nucleotide sequence comprising the BGH polyA signal consists of a sequence set forth in SEQ ID NO: 55.

In one example, the landing pad plasmid comprises a nucleotide sequence encoding one or more additional detectable markers. In one example, the one or more additional detectable markers are not located within the landing pad cassette. For example, the one or more additional detectable markers are located downstream (i.e., 3′) of the 3′ HA of the landing pad cassette. In one example, the one or more additional detectable markers is selected from the group consisting of an enhanced green fluorescent protein (eGFP), a red fluorescent protein (mCherry or mScarlet), a yellow fluorescent protein, a cyan fluorescent protein and combinations thereof. For example, the additional detectable marker is an cGFP. In one example, the additional detectable marker is a mCherry. In one example, the additional detectable marker is an mScarlet. In one example, the additional detectable marker is a yellow fluorescent protein. In one example, the additional detectable marker is a cyan fluorescent protein.

In one example, the landing pad plasmid comprises a nucleotide sequence comprising an additional polyA signal. In one example, the additional polyA signal is not located within the landing pad cassette. For example, the additional poly A are located downstream (i.e., 3′) of the 3′ HA of the landing pad cassette. In one example, the additional polyA signal is a SV40 polyA. SVLP polyA, hGH polyA. BGH polyA or rbGlob polyA. For example, the additional polyA signal is a SV40 polyA. In one example, the additional polyA signal is a SVLP poly A. In one example, the additional polyA signal is a hGH polyA. In one example, the additional polyA signal is a BGH polyA. In one example, the additional polyA signal is a rbGlob poly A. In one example, the landing pad plasmid comprises a nucleotide sequence comprising a viral origin of replication sequence. For example, the viral origin of replication sequence is pUC.

In one example, the landing pad plasmid comprises a nucleotide sequence encoding an antibiotic resistance gene operably linked to a nucleotide sequence comprising a promoter. For example, the antibiotic resistance gene is an Amp (R) gene. In one example, the promoter is a bla promoter.

In one example, the additional promoter is located 3′ of the landing pad cassette 3′ HA and is operably linked to the additional detectable marker. In one example, the additional polyA signal is located 3′ of the additional detectable marker. In one example, the viral origin of replication sequence is located 3′ of the additional polyA signal. In one example, the antibiotic resistance gene operably linked to the promoter are located 3′ of the viral origin of replication sequence.

In one example, the landing pad plasmid comprises, in order from 5′ to 3′, an additional promoter operably linked to the additional detectable marker, an additional polyA signal, a viral origin of replication sequence, and an antibiotic resistance gene operably linked to a promoter, wherein the additional promoter operably linked to the additional detectable marker is located 3′ of the landing pad cassette 3′ HA.

In one example, the landing pad plasmid comprises, in order from 5′ to 3′, the landing pad cassette, an additional promoter operably linked to the additional detectable marker, an additional poly A signal, a viral origin of replication sequence, and an antibiotic resistance gene operably linked to a promoter.

In one example, the nucleotide sequence comprising the 5′ HA comprises or consists of a sequence set forth in SEQ ID NO: 24 or 26. In one example, the nucleotide sequence comprising the 5′HA comprises of a sequence set forth in SEQ ID NO: 24 or 26. In one example, the nucleotide sequence comprising the 5′HA consists of a sequence set forth in SEQ ID NO: 24 or 26.

In one example, the nucleotide sequence comprising the 5′ HA comprises or consists of a sequence set forth in SEQ ID NO: 24. In one example, the nucleotide sequence comprising the 5′HA comprises of a sequence set forth in SEQ ID NO: 24. In one example, the nucleotide sequence comprising the 5′HA consists of a sequence set forth in SEQ ID NO: 24.

In one example, the nucleotide sequence comprising the 5′ HA comprises or consists of a sequence set forth in SEQ ID NO: 26. In one example, the nucleotide sequence comprising the 5′HA comprises of a sequence set forth in SEQ ID NO: 26. In one example, the nucleotide sequence comprising the 5′HA consists of a sequence set forth in SEQ ID NO: 26.

In one example, the nucleotide sequence comprising the 3′ HA comprises or consists of a sequence set forth in SEQ ID NO: 25 or 27. In one example, the nucleotide sequence comprising the 3′HA comprises of a sequence set forth in SEQ ID NO: 25 or 27. In one example, the nucleotide sequence comprising the 3′HA consists of a sequence set forth in SEQ ID NO: 25 or 27.

In one example, the nucleotide sequence comprising the 3′ HA comprises or consists of a sequence set forth in SEQ ID NO: 25. In one example, the nucleotide sequence comprising the 3′HA comprises of a sequence set forth in SEQ ID NO: 25. In one example, the nucleotide sequence comprising the 3′HA consists of a sequence set forth in SEQ ID NO: 25.

In one example, the nucleotide sequence comprising the 3′ HA comprises or consists of a sequence set forth in SEQ ID NO: 27. In one example, the nucleotide sequence comprising the 3′HA comprises of a sequence set forth in SEQ ID NO: 27. In one example, the nucleotide sequence comprising the 3′HA consists of a sequence set forth in SEQ ID NO: 27.

In one example, the nucleotide sequence comprising the 5′ HA comprises or consists of a sequence set forth in SEQ ID NO: 24 and the nucleotide sequence comprising the 3′ HA comprises or consists of a sequence set forth in SEQ ID NO: 25. In one example, the nucleotide sequence comprising the 5′HA comprises of a sequence set forth in SEQ ID NO: 24 and the nucleotide sequence comprising the 3′ HA comprises of a sequence set forth in SEQ ID NO: 25. In one example, the nucleotide sequence comprising the 5′HA consists of a sequence set forth in SEQ ID NO: 24 and the nucleotide sequence comprising the 3′ HA consists of a sequence set forth in SEQ ID NO: 25.

In one example, the nucleotide sequence comprising the 5′ HA comprises or consists of a sequence set forth in SEQ ID NO: 26 and the nucleotide sequence comprising the 3′ HA comprises or consists of a sequence set forth in SEQ ID NO: 27. In one example, the nucleotide sequence comprising the 5′HA comprises of a sequence set forth in SEQ ID NO: 26 and the nucleotide sequence comprising the 3′ HA comprises of a sequence set forth in SEQ ID NO: 27. In one example, the nucleotide sequence comprising the 5′HA consists of a sequence set forth in SEQ ID NO: 26 and the nucleotide sequence comprising the 3′ HA consists of a sequence set forth in SEQ ID NO: 27.

In one example, the landing pad plasmid comprises or consists of a sequence set forth in SEQ ID NO: 1 or 2. In one example, the landing pad plasmid comprises of a sequence set forth in SEQ ID NO: 1 or 2. In one example, the landing pad plasmid consists of a sequence set forth in SEQ ID NO: 1 or 2.

In one example, the landing pad plasmid comprises or consists of a sequence set forth in SEQ ID NO: 1. In one example, the landing pad plasmid comprises of a sequence set forth in SEQ ID NO: 1. In one example, the landing pad plasmid consists of a sequence set forth in SEQ ID NO: 1.

In one example, the landing pad plasmid comprises or consists of a sequence set forth in SEQ ID NO: 2. In one example, the landing pad plasmid comprises of a sequence set forth in SEQ ID NO: 2. In one example, the landing pad plasmid consists of a sequence set forth in SEQ ID NO: 2

In one example, the landing pad plasmid comprises, in order from 5′ to 3′: a) a nucleotide sequence comprising a 5′ homology arm (HA); b) a nucleotide sequence comprising an attP (GT) recombination site: c) a nucleotide sequence comprising a CMV promoter or a nucleotide sequence comprising a MND promoter; d) a nucleotide sequence encoding an enhanced green fluorescent protein (eGFP) detectable marker; c) a nucleotide sequence encoding a P2A self-cleaving peptide linker, or a nucleotide sequence encoding a T2A self-cleaving peptide linker; f) a nucleotide sequence encoding an Herpes Simplex Virus-1 thymidine kinase (HSV-TK) suicide marker; g) a nucleotide sequence comprising a WPRE; h) a nucleotide sequence comprising a BGH polyA signal; i) a nucleotide sequence comprising an attP (GA) recombination site; j) a nucleotide sequence comprising a 3′ HA; k) optionally a nucleotide sequence encoding a mCherry detectable marker; I) a nucleotide sequence comprising a pUC viral origin of replication sequence; and m) a nucleotide sequence encoding an Amp (R) gene operably linked to a nucleotide sequence comprising a bla promoter.

In one example, the landing pad plasmid comprises, in order from 5′ to 3′: a) a nucleotide sequence comprising a 5′ homology arm (HA); b) a nucleotide sequence comprising an attP (GT) recombination site; c) a nucleotide sequence comprising a CMV promoter; d) a nucleotide sequence encoding an enhanced green fluorescent protein (eGFP) detectable marker; e) a nucleotide sequence encoding a P2A self-cleaving peptide linker, or a nucleotide sequence encoding a T2A self-cleaving peptide linker; f) a nucleotide sequence encoding an Herpes Simplex Virus-1 thymidine kinase (HSV-TK) suicide marker; g) a nucleotide sequence comprising a WPRE; h) a nucleotide sequence comprising a BGH polyA signal; i) a nucleotide sequence comprising an attP (GA) recombination site; j) a nucleotide sequence comprising a 3′ HA; k) optionally a nucleotide sequence encoding a mCherry detectable marker; 1) a nucleotide sequence comprising a pUC viral origin of replication sequence; and m) a nucleotide sequence encoding an Amp (R) gene operably linked to a nucleotide sequence comprising a bla promoter.

In one example, the landing pad plasmid comprises, in order from 5′ to 3′: a) a nucleotide sequence comprising a 5′ homology arm (HA); b) a nucleotide sequence comprising an attP (GT) recombination site; c) a nucleotide sequence comprising a MND promoter; d) a nucleotide sequence encoding an enhanced green fluorescent protein (eGFP) detectable marker; e) a nucleotide sequence encoding a P2A self-cleaving peptide linker, or a nucleotide sequence encoding a T2A self-cleaving peptide linker; f) a nucleotide sequence encoding an Herpes Simplex Virus-1 thymidine kinase (HSV-TK) suicide marker; g) a nucleotide sequence comprising a WPRE; h) a nucleotide sequence comprising a BGH polyA signal; i) a nucleotide sequence comprising an attP (GA) recombination site; j) a nucleotide sequence comprising a 3′ HA; k) optionally a nucleotide sequence encoding a mCherry detectable marker; I) a nucleotide sequence comprising a pUC viral origin of replication sequence; and m) a nucleotide sequence encoding an Amp (R) gene operably linked to a nucleotide sequence comprising a bla promoter.

In one example, the landing pad plasmid comprises, in order from 5′ to 3′: a) a nucleotide sequence comprising a 5′ homology arm (HA) comprising a sequence set forth in SEQ ID NO: 24 or 26; b) a nucleotide sequence comprising an attP (GT) recombination site; c) a nucleotide sequence comprising a CMV promoter comprising a sequence set forth in SEQ ID NO: 3 or 4, or a nucleotide sequence comprising a MND promoter comprising a sequence set forth in SEQ ID NO: 5; d) a nucleotide sequence encoding an enhanced green fluorescent protein (cGFP) detectable marker comprising a sequence set forth in SEQ ID NO: 7; c) a nucleotide sequence encoding a P2A self-cleaving peptide linker comprising a sequence set forth in any one of SEQ ID NOs: 10 to 13, or a nucleotide sequence encoding a T2A self-cleaving peptide linker comprising a sequence set forth in SEQ ID NO: 15 or 16; f) a nucleotide sequence encoding an Herpes Simplex Virus-1 thymidine kinase (HSV-TK) suicide marker comprising a sequence set forth in SEQ ID NO: 6; g) a nucleotide sequence comprising a WPRE comprising a sequence set forth in SEQ ID NO: 20; h) a nucleotide sequence comprising a BGH polyA signal; i) a nucleotide sequence comprising an attP (GA) recombination site; j) a nucleotide sequence comprising a 3′ HA comprising a sequence set forth in SEQ ID NO: 25 or 27; k) optionally a nucleotide sequence encoding a mCherry detectable marker: 1) a nucleotide sequence comprising a pUC viral origin of replication sequence; and m) a nucleotide sequence encoding an Amp (R) gene operably linked to a nucleotide sequence comprising a bla promoter.

In one example, the landing pad plasmid comprises, in order from 5′ to 3′: a) a nucleotide sequence comprising a 5′ homology arm (HA); b) a nucleotide sequence comprising an attP (GT) recombination site; c) a nucleotide sequence comprising a MND promoter; d) a nucleotide sequence encoding an enhanced green fluorescent protein (eGFP) detectable marker; c) a nucleotide sequence encoding a P2A self-cleaving peptide linker f) a nucleotide sequence encoding a puromycin N-acetyl-transferase selection marker g) a nucleotide sequence encoding a T2A self-cleaving peptide linker; h) a nucleotide sequence encoding an Herpes Simplex Virus-1 thymidine kinase (HSV-TK) suicide marker; i) a nucleotide sequence comprising a woodchuck hepatitis virus post-transcriptional regulatory element (WPRE); j) a nucleotide sequence comprising a BGH polyA signal; k) a nucleotide sequence comprising an attP (GA) recombination site; 1) a nucleotide sequence comprising a 3′ HA; m) a nucleotide sequence comprising a pUC viral origin of replication sequence; and n) a nucleotide sequence encoding an Amp (R) gene operably linked to a nucleotide sequence comprising a bla promoter.

In one example, the landing pad plasmid has a peDNA3.1 backbone.

The present disclosure also provides a method of stably integrating the landing pad cassette as described herein into the genome of a cell at a specific locus. In one example, the method comprises transfecting the landing pad cassette as described herein into the cell line in a cell culture. For example, the cell culture comprises cells and media.

In one example, the specific locus is associated with adverse events or genotoxicity. For example, the specific locus is a leukemia oncogene (LMO2) locus, a MDS1 And EVI1 Complex (MECOM) locus, a cyclin D2 (CCND2) locus. B lymphoma Mo-MLV insertion region 1 homolog (BMI1) locus or a meningioma (disrupted in balanced translocation) 1 (MN1) locus. In one example, the specific locus is a LMO2 locus. In one example, the specific locus is a MECOM locus. In one example, the specific locus is a CCND2 locus. In one example, the specific locus is a BMI1 locus. In one example, the specific locus is a MN1 locus.

In one example, the LMO2 locus is 33 kb upstream of the transcription start site (TSS) or 2 kb downstream of the TSS. In one example, the LMO2 locus is 38 kb upstream of the TSS. In one example, the LMO2 locus is 37 kb upstream of the TSS. In one example, the LMO2 locus is 36 kb upstream of the TSS. In one example, the LMO2 locus is 35 kb upstream of the TSS. In one example, the LMO2 locus is 34 kb upstream of the TSS. In one example, the LMO2 locus is 33 kb upstream of the TSS. In one example, the LMO2 locus is 32 kb upstream of the TSS. In one example, the LMO2 locus is 31 kb upstream of the TSS. In one example, the LMO2 locus is 30 kb upstream of the TSS. In one example, the LMO2 locus is 29 kb upstream of the TSS. In one example, the LMO2 locus is 28 kb upstream of the TSS. In one example, the LMO2 locus is 1 kb downstream of the TSS. In one example, the LMO2 locus is 2 kb downstream of the TSS. In one example, the LMO2 locus is 3 kb downstream of the TSS. In one example, the LMO2 locus is 4 kb downstream of the TSS. In one example, the LMO2 locus is 5 kb downstream of the TSS. In one example, the LMO2 locus is located between 5 kb downstream of the TSS and 33 kb upstream of the TSS. For example, the LMO2 locus is located between 2 kb downstream of the TSS and 38 kb upstream of the TSS.

In one example, the specific locus is a safe-harbour locus, a common integration site (CIS) or other desired locus within the genome. For example, the specific locus is safe-harbour locus selected from an AAV integration site 1 (AAVS1), a Hypoxanthinc Phosphoribosyltransferase 1 (HPRT) locus, an albumin locus, a hROSA26 locus or a chemokine (CC motif) receptor 5 (CCR5) locus or for example, the specific locus is a common integration site. For example, the specific locus is a common integration site (CIS) selected from Caspase Recruitment Domain Family Member 8 (CARD8). Nuclear Receptor Binding SET Domain Protein 1 (NSD1), Glutamine Rich Protein 1 (QRICH1), SAPS domain family, member 2 (SAPS2), Ubiquitin Specific Peptidase 48 (USP48), G-Patch Domain Containing 8 (GPATCH8), FCH And Double SH3 Domains 2 (FCHSD2), Nuclear protein localization protein 4 (NPLOC4). SWI/SNF Related. Matrix Associated. Actin Dependent Regulator Of Chromatin Subfamily C Member 1 (SMARCC1), Neurofibromatosis 1 (NF1), Phosphofurin acidic cluster sorting protein 1 (PACS1), human leukocyte antigen (HLA), or F-box and leucine-rich repeat 11 (FBXL.11).

In one example, the landing pad cassette is integrated into the genome of a cell at a specific locus using site-directed modification. For example, the landing pad cassette is integrated into the genome of a cell at a specific locus using homology directed recombination (HDR). For example, the HDR utilises zinc-finger nucleases, CRISPR/Cas9 mediated targeting. Cre-Lox. Flp-FRT or transcription activator-like effector nucleases (TALEN). In one example the HDR utilises zinc-finger nucleases. In one example the HDR utilises CRISPR/Cas9 mediated targeting. In one example the HDR utilises Cre-Lox. In one example the HDR utilises Flp-FRT. In one example the HDR utilises transcription activator-like effector nucleases (TALEN).

In one example, the cell is a mammalian cell. For example, the cell is a Jurkat cell, a HEK293 cell, a HEK293T cell, a HEK293T/17 cell, a GPRG cell, a GPRTG cell, a K562 cell, a U-937 cell, a CHO cell, a hematopoietic progenitor or stem cell (e.g. CD34+ cell), a monocyte, a macrophage, a peripheral blood mononuclear cell, a hepatocyte, a CD4+T lymphocyte, a CD8+T lymphocyte, a dendritic cell, or a derivative thereof.

In one example, the method comprises selecting the cells comprising the landing pad cassette (i.e., selecting cells into which the landing pad cassette has been integrated). For example, selecting the cells comprising the landing pad cassette comprises selecting the cells expressing the detectable marker by fluorescence-activated cell sorting and selecting the cells resistant to antibiotic treatment. In one example, selecting the cells comprising the landing pad cassette comprises selecting the cells expressing the detectable marker by fluorescence-activated cell sorting. In one example, selecting the cells comprising the landing pad cassette comprises selecting the cells resistant to antibiotic treatment. In one example, the antibiotic treatment is G418. Hygromycin B. Puromycin. Zeocin. Blasticidin S or L-Histidinol. In one example, the antibiotic treatment is G418. In one example, the antibiotic treatment is Hygromycin B. In one example, the antibiotic treatment is Puromycin. In one example, the antibiotic treatment is Zeocin. In one example, the antibiotic treatment is Blasticidin S. In one example, the antibiotic treatment is L-Histidinol.

The present disclosure provides a cell having the landing pad cassette of the disclosure stably integrated therein, e.g., into its genome.

The disclosure further provides a population of cells, each cell having the landing pad cassette of the disclosure stably integrated therein, e.g., into its genome. In one example, the population of cells are clones.

In one example, the disclosure provides a stable cell line, each cell having the landing pad cassette of the disclosure stably integrated into its genome.

In one example, the cells comprising the landing pad cassette are expanded to produce a stable cell line. For example, the present disclosure provides a stable cell line comprising the landing pad cassette as described herein.

The present disclosure further provides a provirus construct comprising: a) a nucleotide sequence comprising one of more site-specific recombination sites; b) a nucleotide sequence comprising a 5′ long terminal repeat (LTR); c) a nucleotide sequence encoding a transgene of interest operably linked to a promoter; and d) a nucleotide sequence comprising a 3′ LTR comprising an insulator.

In one example, the insulator is in a forward orientation relative to a nucleotide sequence encoding a transgene of interest, wherein the insulator is on the same strand of a nucleic acid as the transgene. In one example, the insulator is in a reverse orientation relative to a nucleotide sequence encoding a transgene of interest, wherein the insulator is on the same strand of a nucleic acid as the transgene.

For example, the provirus construct comprises an insulator in a forward orientation relative to a nucleotide sequence encoding a transgene of interest, wherein the insulator is on the same strand of a nucleic acid as the transgene. In one example, the provirus construct comprises an insulator in a reverse orientation relative to a nucleotide sequence encoding a transgene of interest, wherein the insulator is on the same strand of a nucleic acid as the transgene.

In one example, the insulator is selected from a chicken hypersensitive site-4 (cHS4) insulator, an A1 insulator, an A2 insulator, a C1 insulator, a B4 insulator, a 22-3 insulator, a FB insulator, a foamy virus (FV) insulator, a Sns5 insulator, and combinations thereof. For example, the insulator may be selected from a cHS4 400 bp insulator, a cHS4 650 bp insulator, a cHS4 1200 bp insulator, an A1 300 bp insulator, an A2 266 bp insulator, a C1 325 bp insulator, a B4 260 bp insulator, a 22-3 238 bp insulator, a FB 77 bp insulator, a Foamy Virus (FV) 36 bp insulator, a Sns5 458 bp insulator, and combinations thereof.

In one example, the insulator in a forward orientation relative to a nucleotide sequence encoding a transgene of interest comprises or consists of a sequence set forth in any one of SEQ ID NOs: 28, 29, 32 to 38, 58, 59 and 69.

In one example, the insulator in a reverse orientation relative to a nucleotide sequence encoding a transgene of interest comprises or consists of a sequence set forth in any one of SEQ ID NOs: 30, 31, 56, 57, 60 to 68 and 70.

In one example, the insulator comprises or consists of a sequence set forth in any one of SEQ ID NOs: 32 to 37, 49 and 50. In one example, the insulator comprises of a sequence set forth in any one of SEQ ID NOs: 32 to 37, 49 and 50. In one example, the insulator consists of a sequence set forth in any one of SEQ ID NOs: 32 to 37, 49 and 50.

In one example, the insulator comprises or consists of a sequence set forth in SEQ ID NO: 32. In one example, the insulator comprises of a sequence set forth in SEQ ID NO: 32. In one example, the insulator consists of a sequence set forth in SEQ ID NO: 32.

In one example, the insulator comprises or consists of a sequence set forth in SEQ ID NO: 33. In one example, the insulator comprises of a sequence set forth in SEQ ID NO: 33. In one example, the insulator consists of a sequence set forth in SEQ ID NO: 33.

In one example, the insulator comprises or consists of a sequence set forth in SEQ ID NO: 34. In one example, the insulator comprises of a sequence set forth in SEQ ID NO: 34. In one example, the insulator consists of a sequence set forth in SEQ ID NO: 34.

In one example, the insulator comprises or consists of a sequence set forth in SEQ ID NO: 35. In one example, the insulator comprises of a sequence set forth in SEQ ID NO: 35. In one example, the insulator consists of a sequence set forth in SEQ ID NO: 35.

In one example, the insulator comprises or consists of a sequence set forth in SEQ ID NO: 36. In one example, the insulator comprises of a sequence set forth in SEQ ID NO: 36. In one example, the insulator consists of a sequence set forth in SEQ ID NO: 36.

In one example, the insulator comprises or consists of a sequence set forth in SEQ ID NO: 37. In one example, the insulator comprises of a sequence set forth in SEQ ID NO: 37. In one example, the insulator consists of a sequence set forth in SEQ ID NO: 37.

In one example, the insulator comprises or consists of a sequence set forth in SEQ ID NO: 49. In one example, the insulator comprises of a sequence set forth in SEQ ID NO: 49. In one example, the insulator consists of a sequence set forth in SEQ ID NO: 49.

In one example, the insulator comprises or consists of a sequence set forth in SEQ ID NO: 50. In one example, the insulator comprises of a sequence set forth in SEQ ID NO: 50. In one example, the insulator consists of a sequence set forth in SEQ ID NO: 50.

In one example, the insulator in a forward orientation relative to a nucleotide sequence encoding a transgene of interest comprises or consists of a sequence set forth in SEQ ID NO: 32. In one example, the insulator in a forward orientation relative to a nucleotide sequence encoding a transgene of interest comprises of a sequence set forth in SEQ ID NO: 32. In one example, the insulator in a forward orientation relative to a nucleotide sequence encoding a transgene of interest consists of a sequence set forth in SEQ ID NO: 32.

In one example, the insulator in a forward orientation relative to a nucleotide sequence encoding a transgene of interest comprises or consists of a sequence set forth in SEQ ID NO: 33. In one example, the insulator in a forward orientation relative to a nucleotide sequence encoding a transgene of interest comprises of a sequence set forth in SEQ ID NO: 33. In one example, the insulator in a forward orientation relative to a nucleotide sequence encoding a transgene of interest consists of a sequence set forth in SEQ ID NO: 33.

In one example, the insulator in a forward orientation relative to a nucleotide sequence encoding a transgene of interest comprises or consists of a sequence set forth in SEQ ID NO: 34. In one example, the insulator in a forward orientation relative to a nucleotide sequence encoding a transgene of interest comprises of a sequence set forth in SEQ ID NO: 34. In one example, the insulator in a forward orientation relative to a nucleotide sequence encoding a transgene of interest consists of a sequence set forth in SEQ ID NO: 34.

In one example, the insulator in a forward orientation relative to a nucleotide sequence encoding a transgene of interest comprises or consists of a sequence set forth in SEQ ID NO: 35. In one example, the insulator in a forward orientation relative to a nucleotide sequence encoding a transgene of interest comprises of a sequence set forth in SEQ ID NO: 35. In one example, the insulator in a forward orientation relative to a nucleotide sequence encoding a transgene of interest consists of a sequence set forth in SEQ ID NO: 35.

In one example, the insulator in a forward orientation relative to a nucleotide sequence encoding a transgene of interest comprises or consists of a sequence set forth in SEQ ID NO: 36. In one example, the insulator in a forward orientation relative to a nucleotide sequence encoding a transgene of interest comprises of a sequence set forth in SEQ ID NO: 36. In one example, the insulator in a forward orientation relative to a nucleotide sequence encoding a transgene of interest consists of a sequence set forth in SEQ ID NO: 36.

In one example, the insulator in a forward orientation relative to a nucleotide sequence encoding a transgene of interest comprises or consists of a sequence set forth in SEQ ID NO: 37. In one example, the insulator in a forward orientation relative to a nucleotide sequence encoding a transgene of interest comprises of a sequence set forth in SEQ ID NO: 37. In one example, the insulator in a forward orientation relative to a nucleotide sequence encoding a transgene of interest consists of a sequence set forth in SEQ ID NO: 37.

In one example, the insulator in a forward orientation relative to a nucleotide sequence encoding a transgene of interest comprises or consists of a sequence set forth in SEQ ID NO: 49. In one example, the insulator in a forward orientation relative to a nucleotide sequence encoding a transgene of interest comprises of a sequence set forth in SEQ ID NO: 49. In one example, the insulator in a forward orientation relative to a nucleotide sequence encoding a transgene of interest consists of a sequence set forth in SEQ ID NO: 49.

In one example, the insulator in a forward orientation relative to a nucleotide sequence encoding a transgene of interest comprises or consists of a sequence set forth in SEQ ID NO: 50. In one example, the insulator in a forward orientation relative to a nucleotide sequence encoding a transgene of interest comprises of a sequence set forth in SEQ ID NO: 50. In one example, the insulator in a forward orientation relative to a nucleotide sequence encoding a transgene of interest consists of a sequence set forth in SEQ ID NO: 50.

In one example, the insulator in a reverse orientation relative to a nucleotide sequence encoding a transgene of interest comprises or consists of a sequence set forth in any one of SEQ ID NOs: 60 to 65, 67 and 68. In one example, the insulator in a reverse orientation relative to a nucleotide sequence encoding a transgene of interest comprises of a sequence set forth in any one of SEQ ID NOs: 60 to 65, 67 and 68. In one example, the insulator in a reverse orientation relative to a nucleotide sequence encoding a transgene of interest consists of a sequence set forth in any one of SEQ ID NOs: 60 to 65, 67 and 68.

In one example, the insulator in a reverse orientation relative to a nucleotide sequence encoding a transgene of interest comprises or consists of a sequence set forth in SEQ ID NO: 60. In one example, the insulator in a reverse orientation relative to a nucleotide sequence encoding a transgene of interest comprises of a sequence set forth in SEQ ID NO: 60. In one example, the insulator in a reverse orientation relative to a nucleotide sequence encoding a transgene of interest consists of a sequence set forth in SEQ ID NO: 60.

In one example, the insulator in a reverse orientation relative to a nucleotide sequence encoding a transgene of interest comprises or consists of a sequence set forth in SEQ ID NO:

61. In one example, the insulator in a reverse orientation relative to a nucleotide sequence encoding a transgene of interest comprises of a sequence set forth in SEQ ID NO: 61. In one example, the insulator in a reverse orientation relative to a nucleotide sequence encoding a transgene of interest consists of a sequence set forth in SEQ ID NO: 61.

In one example, the insulator in a reverse orientation relative to a nucleotide sequence encoding a transgene of interest comprises or consists of a sequence set forth in SEQ ID NO: 62. In one example, the insulator in a reverse orientation relative to a nucleotide sequence encoding a transgene of interest comprises of a sequence set forth in SEQ ID NO: 62. In one example, the insulator in a reverse orientation relative to a nucleotide sequence encoding a transgene of interest consists of a sequence set forth in SEQ ID NO: 62.

In one example, the insulator in a reverse orientation relative to a nucleotide sequence encoding a transgene of interest comprises or consists of a sequence set forth in SEQ ID NO: 63. In one example, the insulator in a reverse orientation relative to a nucleotide sequence encoding a transgene of interest comprises of a sequence set forth in SEQ ID NO: 63. In one example, the insulator in a reverse orientation relative to a nucleotide sequence encoding a transgene of interest consists of a sequence set forth in SEQ ID NO: 63.

In one example, the insulator in a reverse orientation relative to a nucleotide sequence encoding a transgene of interest comprises or consists of a sequence set forth in SEQ ID NO: 64. In one example, the insulator in a reverse orientation relative to a nucleotide sequence encoding a transgene of interest comprises of a sequence set forth in SEQ ID NO: 64. In one example, the insulator in a reverse orientation relative to a nucleotide sequence encoding a transgene of interest consists of a sequence set forth in SEQ ID NO: 64.

In one example, the insulator in a reverse orientation relative to a nucleotide sequence encoding a transgene of interest comprises or consists of a sequence set forth in SEQ ID NO: 65. In one example, the insulator in a reverse orientation relative to a nucleotide sequence encoding a transgene of interest comprises of a sequence set forth in SEQ ID NO: 65. In one example, the insulator in a reverse orientation relative to a nucleotide sequence encoding a transgene of interest consists of a sequence set forth in SEQ ID NO: 65.

In one example, the insulator in a reverse orientation relative to a nucleotide sequence encoding a transgene of interest comprises or consists of a sequence set forth in SEQ ID NO: 67. In one example, the insulator in a reverse orientation relative to a nucleotide sequence encoding a transgene of interest comprises of a sequence set forth in SEQ ID NO: 67. In one example, the insulator in a reverse orientation relative to a nucleotide sequence encoding a transgene of interest consists of a sequence set forth in SEQ ID NO: 67.

In one example, the insulator in a reverse orientation relative to a nucleotide sequence encoding a transgene of interest comprises or consists of a sequence set forth in SEQ ID NO: 68. In one example, the insulator in a reverse orientation relative to a nucleotide sequence encoding a transgene of interest comprises of a sequence set forth in SEQ ID NO: 68. In one example, the insulator in a reverse orientation relative to a nucleotide sequence encoding a transgene of interest consists of a sequence set forth in SEQ ID NO: 68. In one example, the insulator is a chicken hypersensitive site-4 (cHS4) insulator.

In one example, the cHS4 insulator is in a forward orientation relative to a nucleotide sequence encoding a transgene of interest, wherein the insulator is on the same strand of a nucleic acid as the transgene. In one example, the cHS4 insulator is in a reverse orientation relative to a nucleotide sequence encoding a transgene of interest, wherein the insulator is on the same strand of a nucleic acid as the transgene.

In one example, the cHS4 insulator in a forward orientation relative to a nucleotide sequence encoding a transgene of interest comprises or consists of a sequence set forth in any one of SEQ ID NOs: 28, 29, 38, 58, 59 and 69. In one example, the cHS4 insulator in a forward orientation relative to a nucleotide sequence encoding a transgene of interest comprises of a sequence set forth in any one of SEQ ID NOs: 28, 29, 38, 58, 59 and 69. In one example, the cHS4 insulator in a forward orientation relative to a nucleotide sequence encoding a transgene of interest consists of a sequence set forth in any one of SEQ ID NOs: 28, 29, 38, 58, 59 and 69.

In one example, the cHS4 insulator in a reverse orientation relative to a nucleotide sequence encoding a transgene of interest comprises or consists of a sequence set forth in any one of SEQ ID NOs: 30, 31, 56, 57, 66 and 70. In one example, the cHS4 insulator in a reverse orientation relative to a nucleotide sequence encoding a transgene of interest comprises of a sequence set forth in any one of SEQ ID NOs: 30, 31, 56, 57, 66 and 70. In one example, the cHS4 insulator in a reverse orientation relative to a nucleotide sequence encoding a transgene of interest consists of a sequence set forth in any one of SEQ ID NOs: 30, 31, 56, 57, 66 and 70.

For example, the cHS4 insulator comprises or consists of a sequence set forth in any one of SEQ ID NOs: 29 to 31 and 38. In one example, the cHS4 insulator comprises of a sequence set forth in any one of SEQ ID NOs: 29 to 31 and 38. In one example, the cHS4 insulator consists of a sequence set forth in any one of SEQ ID NOs: 29 to 31 and 38.

In one example, the cHS4 insulator in a forward orientation relative to a nucleotide sequence encoding a transgene of interest comprises or consists of a sequence set forth in SEQ ID NO: 28. In one example, the cHS4 insulator in a forward orientation relative to a nucleotide sequence encoding a transgene of interest comprises of a sequence set forth SEQ ID NO: 28. In one example, the cHS4 insulator in a forward orientation relative to a nucleotide sequence encoding a transgene of interest consists of a sequence set forth SEQ ID NO: 28. In one example, the cHS4 insulator comprises or consists of a sequence set forth in SEQ ID NO: 29. In one example, the cHS4 insulator comprises of a sequence set forth in SEQ ID NO: 29. In one example, the cHS4 insulator consists of a sequence set forth in SEQ ID NO: 29.

In one example, the cHS4 insulator comprises or consists of a sequence set forth in SEQ ID NO: 30. In one example, the cHS4 insulator comprises of a sequence set forth in SEQ ID NO: 30. In one example, the cHS4 insulator consists of a sequence set forth in SEQ ID NO: 30.

In one example, the cHS4 insulator comprises or consists of a sequence set forth in SEQ ID NO: 31. In one example, the cHS4 insulator comprises of a sequence set forth in SEQ ID NO: 31. In one example, the cHS4 insulator consists of a sequence set forth in SEQ ID NO: 31.

In one example, the cHS4 insulator comprises or consists of a sequence set forth in SEQ ID NO: 38. In one example, the cHS4 insulator comprises of a sequence set forth in SEQ ID NO: 38. In one example, the cHS4 insulator consists of a sequence set forth in SEQ ID NO: 38.

In one example, the cHS4 insulator in a forward orientation relative to a nucleotide sequence encoding a transgene of interest comprises or consists of a sequence set forth in SEQ ID NO: 29. In one example, the cHS4 insulator in a forward orientation relative to a nucleotide sequence encoding a transgene of interest comprises of a sequence set forth in SEQ ID NO: 29. In one example, the cHS4 insulator in a forward orientation relative to a nucleotide sequence encoding a transgene of interest consists of a sequence set forth in SEQ ID NO: 29.

In one example, the cHS4 insulator in a forward orientation relative to a nucleotide sequence encoding a transgene of interest comprises or consists of a sequence set forth in SEQ ID NO: 30. In one example, the cHS4 insulator in a forward orientation relative to a nucleotide sequence encoding a transgene of interest comprises of a sequence set forth in SEQ ID NO: 30. In one example, the cHS4 insulator in a forward orientation relative to a nucleotide sequence encoding a transgene of interest consists of a sequence set forth in SEQ ID NO: 30.

In one example, the cHS4 insulator in a forward orientation relative to a nucleotide sequence encoding a transgene of interest comprises or consists of a sequence set forth in SEQ ID NO: 31. In one example, the cHS4 insulator in a forward orientation relative to a nucleotide sequence encoding a transgene of interest comprises of a sequence set forth in SEQ ID NO: 31. In one example, the cHS4 insulator in a forward orientation relative to a nucleotide sequence encoding a transgene of interest consists of a sequence set forth in SEQ ID NO: 31.

In one example, the cHS4 insulator in a forward orientation relative to a nucleotide sequence encoding a transgene of interest comprises or consists of a sequence set forth in SEQ ID NO: 38. In one example, the cHS4 insulator in a forward orientation relative to a nucleotide sequence encoding a transgene of interest comprises of a sequence set forth in SEQ ID NO: 38. In one example, the cHS4 insulator in a forward orientation relative to a nucleotide sequence encoding a transgene of interest consists of a sequence set forth in SEQ ID NO: 38. For example, the cHS4 insulator comprises or consists of a sequence set forth in any one of SEQ ID NOs: 56 to 59, 66, 69 and 70. In one example, the cHS4 insulator comprises of a sequence set forth in any one of SEQ ID NOs: 56 to 59, 66, 69 and 70. In one example, the cHS4 insulator consists of a sequence set forth in any one of SEQ ID NOs: 56 to 59, 66, 69 and 70.

In one example, the cHS4 insulator in a reverse orientation relative to a nucleotide sequence encoding a transgene of interest comprises or consists of a sequence set forth in SEQ ID NO: 56. In one example, the cHS4 insulator in a reverse orientation relative to a nucleotide sequence encoding a transgene of interest comprises of a sequence set forth in SEQ ID NO: 56. In one example, the cHS4 insulator consists of a sequence set forth in SEQ ID NO: 56.

In one example, the cHS4 insulator in a reverse orientation relative to a nucleotide sequence encoding a transgene of interest comprises or consists of a sequence set forth in SEQ ID NO: 57. In one example, the cHS4 insulator in a reverse orientation relative to a nucleotide sequence encoding a transgene of interest comprises of a sequence set forth in SEQ ID NO: 57. In one example, the cHS4 insulator in a reverse orientation relative to a nucleotide sequence encoding a transgene of interest consists of a sequence set forth in SEQ ID NO: 57.

In one example, the cHS4 insulator in a forward orientation relative to a nucleotide sequence encoding a transgene of interest comprises or consists of a sequence set forth in SEQ ID NO: 58. In one example, the cHS4 insulator in a forward orientation relative to a nucleotide sequence encoding a transgene of interest comprises of a sequence set forth in SEQ ID NO: 58. In one example, the cHS4 insulator in a forward orientation relative to a nucleotide sequence encoding a transgene of interest consists of a sequence set forth in SEQ ID NO: 58.

In one example, the cHS4 insulator in a forward orientation relative to a nucleotide sequence encoding a transgene of interest comprises or consists of a sequence set forth in SEQ ID NO: 59. In one example, the cHS4 insulator in a forward orientation relative to a nucleotide sequence encoding a transgene of interest comprises of a sequence set forth in SEQ ID NO: 59. In one example, the cHS4 insulator in a forward orientation relative to a nucleotide sequence encoding a transgene of interest consists of a sequence set forth in SEQ ID NO: 59.

In one example, the cHS4 insulator in a forward orientation relative to a nucleotide sequence encoding a transgene of interest comprises or consists of a sequence set forth in SEQ ID NO: 66. In one example, the cHS4 insulator in a forward orientation relative to a nucleotide sequence encoding a transgene of interest comprises of a sequence set forth in SEQ ID NO: 66. In one example, the cHS4 insulator in a forward orientation relative to a nucleotide sequence encoding a transgene of interest consists of a sequence set forth in SEQ ID NO: 66.

In one example, the cHS4 insulator in a forward orientation relative to a nucleotide sequence encoding a transgene of interest comprises or consists of a sequence set forth in SEQ ID NO: 69. In one example, the cHS4 insulator in a forward orientation relative to a nucleotide sequence encoding a transgene of interest comprises of a sequence set forth in SEQ ID NO: 69. In one example, the cHS4 insulator in a forward orientation relative to a nucleotide sequence encoding a transgene of interest consists of a sequence set forth in SEQ ID NO: 69. In one example, the cHS4 insulator in a reverse orientation relative to a nucleotide sequence encoding a transgene of interest comprises or consists of a sequence set forth in SEQ ID NO: 70. In one example, the cHS4 insulator in a reverse orientation relative to a nucleotide sequence encoding a transgene of interest comprises of a sequence set forth in SEQ ID NO: 70. In one example, the cHS4 insulator in a reverse orientation relative to a nucleotide sequence encoding a transgene of interest consists of a sequence set forth in SEQ ID NO: 70. In one example, the provirus construct comprises an insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In one example, the provirus construct comprises an insulator in a reverse orientation relative to a nucleotide sequence encoding a transgene of interest, wherein the transgene of interest is selected from treating sickle cell disease (SCD) or Wiskott-Aldrich Syndrome (WAS), and wherein the insulator is on the same strand of a nucleic acid as the transgene.

In one example, the insulator is a cHS4 insulator in a forward orientation relative to a nucleotide sequence encoding a transgene of interest, wherein the transgene of interest is useful for treating sickle cell disease (SCD) or Wiskott-Aldrich Syndrome (WAS). In another example, the insulator is cHS4 insulator in a reverse orientation relative to a nucleotide sequence encoding a transgene of interest, wherein the transgene of interest is useful for treating sickle cell disease (SCD) or Wiskott-Aldrich Syndrome (WAS). In one example, the insulator is 1200 bp cHS4 insulator in a reverse orientation relative to a nucleotide sequence encoding a transgene of interest, wherein the transgene of interest is useful for treating sickle cell disease (SCD) or Wiskott-Aldrich Syndrome (WAS). In one example, the insulator is 250 bp cHS4 insulator in a forward orientation relative to a nucleotide sequence encoding a transgene of interest, wherein the transgene of interest is useful for treating sickle cell disease (SCD) or Wiskott-Aldrich Syndrome (WAS). In one example, the insulator is 400 bp cHS4 insulator in a forward orientation relative to a nucleotide sequence encoding a transgene of interest, wherein the transgene of interest is useful for treating sickle cell disease (SCD) or Wiskott-Aldrich Syndrome (WAS).

In one example, the insulator is an A1 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the insulator is an A1 insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In one example, the insulator is a 300 bp A1 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene.

In one example, the insulator is an A2 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the insulator is an A2 insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In one example, the insulator is a 266 bp A2 insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene.

In one example, the insulator is a C1 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the insulator is a C1 insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In one example, the insulator is a 325 bp C1 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene.

In one example, the insulator is a B4 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the insulator is a B4 insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In one example, the insulator is a 260 bp B4 insulator in a forward orientation relative to a nucleotide sequence encoding a transgene of interest, wherein the transgene of interest is useful for treating sickle cell disease (SCD) or Wiskott-Aldrich

Syndrome (WAS), and wherein the insulator is on the same strand of a nucleic acid as the transgene.

In one example, the insulator is a 22-3 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the insulator is a 22-3 insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In one example, the insulator is a 238 bp 22-3 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene.

In one example, the insulator is a FB insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the insulator is a FB insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In one example, the insulator is a 77 bp FB insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene.

In one example, the insulator is a foamy virus (FV) insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the insulator is a foamy virus (FV) insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the insulator is a 36 bp foamy virus (FV) insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene.

In one example, the insulator is a Sns5 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the insulator is a Sns5 insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the insulator is a 458 bp Sns5 insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene.

In one example, the provirus construct comprises, a nucleotide sequence comprising a MND promoter operably linked to a nucleotide sequence encoding a transgene of interest and an insulator in a forward orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and an insulator in a reverse orientation relative to a nucleotide sequence encoding a transgene of interest, wherein the insulator is on the same strand of a nucleic acid as the transgene.

In one example, the provirus construct comprises, a nucleotide sequence comprising a MND promoter operably linked to a nucleotide sequence encoding a transgene of interest and a cHS4 insulator in a forward orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and a cHS4 insulator in a reverse orientation relative to a nucleotide sequence encoding a transgene of interest, wherein the insulator is on the same strand of a nucleic acid as the transgene.

In one example, the provirus construct comprises, a nucleotide sequence comprising a MND promoter operably linked to a nucleotide sequence encoding a transgene of interest and a 1200 bp cHS4 insulator in a forward orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and a 1200 bp cHS4 insulator in a reverse orientation relative to a nucleotide sequence encoding a transgene of interest, wherein the insulator is on the same strand of a nucleic acid as the transgene.

In one example, the provirus construct comprises, a nucleotide sequence comprising a MND promoter operably linked to a nucleotide sequence encoding a transgene of interest and a 250 bp cHS4 insulator in a forward orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and a 250 bp cHS4 insulator in a reverse orientation relative to a nucleotide sequence encoding a transgene of interest, wherein the insulator is on the same strand of a nucleic acid as the transgene.

In one example, the provirus construct comprises, a nucleotide sequence comprising a MND promoter operably linked to a nucleotide sequence encoding a transgene of interest and a 400 bp cHS4 insulator in a forward orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and a 400 bp cHS4 insulator in a reverse orientation relative to a nucleotide sequence encoding a transgene of interest, wherein the insulator is on the same strand of a nucleic acid as the transgene.

In one example, the provirus construct comprises, a nucleotide sequence comprising a MND promoter operably linked to a nucleotide sequence encoding a transgene of interest and an A1 insulator in a forward orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and an A1 insulator in a reverse orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and a 300 bp A1 insulator in a forward orientation relative to a nucleotide sequence encoding a transgene of interest, wherein the insulator is on the same strand of a nucleic acid as the transgene.

In one example, the provirus construct comprises, a nucleotide sequence comprising a MND promoter operably linked to a nucleotide sequence encoding a transgene of interest and an A2 insulator in a forward orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and an A2 insulator in a reverse orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and a 266 bp A2 insulator in a reverse orientation relative to a nucleotide sequence encoding a transgene of interest, wherein the insulator is on the same strand of a nucleic acid as the transgene.

In one example, the provirus construct comprises, a nucleotide sequence comprising a MND promoter operably linked to a nucleotide sequence encoding a transgene of interest and a C1 insulator in a forward orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and a C1 insulator in a reverse orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and a 325 bp C1 insulator in a forward orientation relative to a nucleotide sequence encoding a transgene of interest, wherein the insulator is on the same strand of a nucleic acid as the transgene.

In one example, the provirus construct comprises, a nucleotide sequence comprising a MND promoter operably linked to a nucleotide sequence encoding a transgene of interest and a B4 insulator in a forward orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and a B4 insulator in a reverse orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and a 260 bp B4 insulator in a forward orientation relative to a nucleotide sequence encoding a transgene of interest, wherein the insulator is on the same strand of a nucleic acid as the transgene.

In one example, the provirus construct comprises, a nucleotide sequence comprising a MND promoter operably linked to a nucleotide sequence encoding a transgene of interest and a 22-3 insulator in a forward orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and a 22-3 insulator in a reverse orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and a 238 bp 22-3 insulator in a forward orientation relative to a nucleotide sequence encoding a transgene of interest, wherein the insulator is on the same strand of a nucleic acid as the transgene.

In one example, the provirus construct comprises, a nucleotide sequence comprising a MND promoter operably linked to a nucleotide sequence encoding a transgene of interest and a FB insulator in a forward orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and a FB insulator in a reverse orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and a 77 bp FB insulator in a forward orientation relative to a nucleotide sequence encoding a transgene of interest, wherein the insulator is on the same strand of a nucleic acid as the transgene.

In one example, the provirus construct comprises, a nucleotide sequence comprising a MND promoter operably linked to a nucleotide sequence encoding a transgene of interest and a foamy virus (FV) insulator in a forward orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and a foamy virus (FV) insulator in a reverse orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and a 36 bp foamy virus (FV) insulator in a forward orientation relative to a nucleotide sequence encoding a transgene of interest, wherein the insulator is on the same strand of a nucleic acid as the transgene.

In one example, the provirus construct comprises, a nucleotide sequence comprising a MND promoter operably linked to a nucleotide sequence encoding a transgene of interest and a Sns5 insulator in a forward orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and a Sns5 insulator in a reverse orientation relative to a nucleotide sequence encoding a transgene of interest, wherein the insulator is on the same strand of a nucleic acid as the transgene. In another example, the provirus construct comprises, a nucleotide sequence comprising a MND promoter operably linked to a nucleotide sequence encoding a transgene of interest and a 458 bp Sns5 insulator in a reverse orientation relative to a nucleotide sequence encoding a transgene of interest, wherein the insulator is on the same strand of a nucleic acid as the transgene.

In one example, the provirus construct comprises, a nucleotide sequence comprising a MND promoter operably linked to a nucleotide sequence encoding a transgene of interest and an insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and an insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene.

In one example, the provirus construct comprises, a nucleotide sequence comprising a MND promoter operably linked to a nucleotide sequence encoding a transgene of interest and a cHS4 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and a cHS4 insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene.

In one example, the provirus construct comprises, a nucleotide sequence comprising a MND promoter operably linked to a nucleotide sequence encoding a transgene of interest and a 1200 bp cHS4 insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and 250 bp cHS4 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and 400 bp cHS4 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene.

In one example, the provirus construct comprises, a nucleotide sequence comprising a MND promoter operably linked to a nucleotide sequence encoding a transgene of interest and an A1 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and an A1 insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and a 300 bp A1 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene.

In one example, the provirus construct comprises, a nucleotide sequence comprising a MND promoter operably linked to a nucleotide sequence encoding a transgene of interest and an A2 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and an A2 insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and a 266 bp A2 insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene.

In one example, the provirus construct comprises, a nucleotide sequence comprising a MND promoter operably linked to a nucleotide sequence encoding a transgene of interest and a B4 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and a B4 insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and a 260 bp B4 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene.

In one example, the provirus construct comprises, a nucleotide sequence comprising a MND promoter operably linked to a nucleotide sequence encoding a transgene of interest and a C1 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and a C1 insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and a 325 bp C1 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene.

In one example, the provirus construct comprises, a nucleotide sequence comprising a MND promoter operably linked to a nucleotide sequence encoding a transgene of interest and a 22-3 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and a 22-3 insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and a 238 bp 22-3 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene.

In one example, the provirus construct comprises, a nucleotide sequence comprising a MND promoter operably linked to a nucleotide sequence encoding a transgene of interest and a FB insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and a FB insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and a 77 bp FB insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene.

In one example, the provirus construct comprises, a nucleotide sequence comprising a MND promoter operably linked to a nucleotide sequence encoding a transgene of interest and a foamy virus (FV) insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and a foamy virus (FV) insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and a 36 bp foamy virus (FV) insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene.

In one example, the provirus construct comprises, a nucleotide sequence comprising a MND promoter operably linked to a nucleotide sequence encoding a transgene of interest and a Sns-5 insulator in a forward orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and a Sns-5 insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene. In another example, the provirus construct comprises, a nucleotide sequence comprising a MND promoter operably linked to a nucleotide sequence encoding a transgene of interest and a 458 bp Sns5 insulator in a reverse orientation relative to a nucleotide sequence encoding a transgene of interest, wherein the transgene of interest is useful for treating sickle cell disease (SCD) or Wiskott-Aldrich Syndrome (WAS), and wherein the insulator is on the same strand of a nucleic acid as the transgene.

In one example, the provirus construct comprises a nucleotide sequence encoding a transgene of interest operably linked to a promoter. In one example, the promoter is a CMV promoter, a CMV enhancer, a MND promoter, a SV40 promoter with enhancer, a UBC promoter, a PGK promoter, an EF1A promoter, a hACTB promoter or a CAG promoter. For example, the promoter is a CMV promoter. In one example, the promoter is a CMV enhancer. In one example, the promoter is a MND promoter. In one example, the promoter is a SV40 promoter with enhancer. In one example, the promoter is a UBC promoter. In one example, the promoter is a PGK promoter. In one example, the promoter is a EFIA promoter. In one example, the promoter is a hACTB promoter. In one example, the promoter is a CAG promoter.

In one example, the nucleotide sequence comprising the MND promoter comprises or consists of a sequence set forth in SEQ ID NO: 39. In one example, the nucleotide sequence comprising the MND promoter comprises of a sequence set forth in SEQ ID NO: 39. In one example, the nucleotide sequence comprising the MND promoter consists of a sequence set forth in SEQ ID NO: 39.

In one example, the nucleotide sequence comprising the 7SK promoter comprises or consists of a sequence set forth in SEQ ID NO: 53. In one example, the nucleotide sequence comprising the 7SK promoter comprises of a sequence set forth in SEQ ID NO: 53. In one example, the nucleotide sequence comprising the 7SK promoter consists of a sequence set forth in SEQ ID NO: 53.

In one example, the nucleotide sequence comprising the 7SK promoter alternative comprises or consists of a sequence set forth in SEQ ID NO: 54. In one example, the nucleotide sequence comprising the 7SK promoter alternative comprises of a sequence set forth in SEQ ID NO: 54. In one example, the nucleotide sequence comprising the 7SK promoter alternative consists of a sequence set forth in SEQ ID NO: 54.

In one example, the provirus construct comprises a nucleotide sequence comprising a woodchuck hepatitis virus post-transcriptional regulatory element (WPRE). For example, the WPRE comprises or consists of a sequence set forth in SEQ ID NO: 41. In one example, the nucleotide sequence comprising the WPRE comprises of a sequence set forth in SEQ ID NO: 41. In one example, the nucleotide sequence comprising the WPRE consists of a sequence set forth in SEQ ID NO: 41.

In one example, the provirus construct comprises a nucleotide sequence comprising a short hairpin RNA 734 (shrna734). For example, the shrna734 comprises or consists of a sequence set forth in SEQ ID NO: 51. In one example, the nucleotide sequence comprising the shrna734 comprises of a sequence set forth in SEQ ID NO: 51. In one example, the nucleotide sequence comprising the shrna734 consists of a sequence set forth in SEQ ID NO: 51. In another example, the shrna734 comprises or consists of a sequence set forth in SEQ ID NO: 52. In one example, the nucleotide sequence comprising the shrna734 comprises of a sequence set forth in SEQ ID NO: 52. In one example, the nucleotide sequence comprising the shrna734 consists of a sequence set forth in SEQ ID NO: 52.

In one example, the provirus construct comprises, in order from 5′ to 3′: a) a nucleotide sequence comprising a first site-specific recombination site, wherein the first site-specific recombination site is an attB (GT) recombination site; b) a nucleotide sequence comprising a 5′ LTR; c) a nucleotide sequence encoding a transgene of interest operably linked to a promoter, wherein the promoter is a CMV promoter, a CMV enhancer, a MND promoter, a SV40 promoter with enhancer, a UBC promoter, a PGK promoter, a EFIA promoter, a hACTB promoter or a CAG promoter; d) a nucleotide comprising a woodchuck hepatitis virus post-transcriptional regulatory element (WPRE); e) a nucleotide sequence comprising a 3′ LTR comprising an insulator, wherein the insulator is in a forward orientation relative to the nucleotide sequence encoding the transgene of interest; and f) a nucleotide sequence comprising a second site-specific recombination site, wherein the second site-specific recombination site is an attB (GA) recombination site.

In one example, the provirus construct comprises, in order from 5′ to 3′: a) a nucleotide sequence comprising a first site-specific recombination site, wherein the first site-specific recombination site is an attB (GT) recombination site; b) a nucleotide sequence comprising a 5′ LTR; c) a nucleotide sequence encoding a transgene of interest operably linked to a promoter, wherein the promoter is a CMV promoter, a CMV enhancer, a MND promoter, a SV40 promoter with enhancer, a UBC promoter, a PGK promoter, a EFIA promoter, a hACTB promoter or a CAG promoter; d) a nucleotide comprising a woodchuck hepatitis virus post-transcriptional regulatory element (WPRE); e) a nucleotide sequence comprising a 3′ LTR comprising an insulator, wherein the insulator is in a reverse orientation relative to the nucleotide sequence encoding the transgene of interest; and f) a nucleotide sequence comprising a second site-specific recombination site, wherein the second site-specific recombination site is an attB (GA) recombination site.

In one example, the provirus construct comprises, in order from 5′ to 3′: a) a nucleotide sequence comprising a first site-specific recombination site, wherein the first site-specific recombination site is an attB (GT) recombination site; b) a nucleotide sequence comprising a 5′ LTR; c) a nucleotide sequence encoding a transgene of interest operably linked to a promoter, wherein the promoter is a CMV promoter, a CMV enhancer, a MND promoter, a SV40 promoter with enhancer, a UBC promoter, a PGK promoter, a EFIA promoter, a hACTB promoter or a CAG promoter; d) a nucleotide comprising a woodchuck hepatitis virus post-transcriptional regulatory element (WPRE); e) a nucleotide sequence comprising a 3′ LTR comprising an insulator, wherein the insulator is a chicken hypersensitive site-4 (cHS4) insulator and optionally wherein the nucleotide sequence comprising the cHS4 insulator comprises a sequence set forth in any one of SEQ ID NOs: 28 to 31, 56 to 59, 66, 69 and 70; and f) a nucleotide sequence comprising a second site-specific recombination site, wherein the second site-specific recombination site is an attB (GA) recombination site.

In one example, the provirus construct comprises, in order from 5′ to 3′: a) a nucleotide sequence comprising a first site-specific recombination site, wherein the first site-specific recombination site is an attB (GT) recombination site; b) a nucleotide sequence comprising a 5′ LTR; c) a nucleotide sequence encoding a transgene of interest operably linked to a promoter, wherein the promoter is a CMV promoter, a CMV enhancer, a MND promoter, a

SV40 promoter with enhancer, a UBC promoter, a PGK promoter, a EFIA promoter, a hACTB promoter or a CAG promoter; d) a nucleotide comprising a woodchuck hepatitis virus post-transcriptional regulatory element (WPRE); c) a nucleotide sequence comprising a 3′ LTR comprising an insulator, wherein the insulator is a chicken hypersensitive site-4 (cHS4) insulator and optionally wherein the nucleotide sequence comprising the cHS4 insulator comprises a sequence set forth in any one of SEQ ID NOs: 29 to 31; and f) a nucleotide sequence comprising a second site-specific recombination site, wherein the second site-specific recombination site is an attB (GA) recombination site.

In one example, the transgene of interest is a detectable marker. For example, the detectable marker is an enhanced green fluorescent protein (eGFP), a red fluorescent protein (mCherry or mScarlet), a yellow fluorescent protein or a cyan fluorescent protein. For example, the detectable marker is an eGFP. In one example, the detectable marker is a mCherry. In one example, the detectable marker is a mScarlet. In one example, the detectable marker is a yellow fluorescent protein. In one example, the detectable marker is a cyan fluorescent protein.

In one example, the transgene of interest is mScarlet. For example, nucleotide sequence encoding the mScarlet comprises or consists of a sequence set forth in SEQ ID NO: 40. In one example, the nucleotide sequence encoding the mScarlet comprises a sequence set forth in SEQ ID NO: 40. In one example, the nucleotide sequence encoding the mScarlet consists of a sequence set forth in SEQ ID NO: 40.

In one example, the transgene of interest is a protein useful for treating a hemoglobinopathy, e.g., sickle cell disease or a thalassemia. In some examples, the transgene of interest is a protein useful for treating a primary immunodeficiency. In one example, the transgene of interest is a protein useful for treating Wiskott-Aldrich Syndrome. In one example, the transgene of interest is a protein useful for treating X linked agammaglobulinemia.

In one example, the provirus construct comprises, in order from 5′ to 3′: a) a nucleotide sequence comprising an attB (GT) recombination site; b) a nucleotide sequence comprising a 5′ LTR: c) lentiviral elements; d) a nucleotide sequence comprising a MND promoter operably linked to a nucleotide sequence encoding a transgene of interest; e) a nucleotide sequence comprising a woodchuck hepatitis virus post-transcriptional regulatory element (WPRE); f) a nucleotide sequence comprising a 3′ LTR comprising an insulator; and g) a nucleotide sequence comprising an attB (GA) recombination site.

In one example, the provirus construct comprises, in order from 5′ to 3′: a) a nucleotide sequence comprising an attB (GT) recombination site; b) a nucleotide sequence comprising a 5′ LTR: c) lentiviral elements; d) a nucleotide sequence comprising a MND promoter operably linked to a nucleotide sequence encoding a transgene of interest; e) a nucleotide sequence comprising a woodchuck hepatitis virus post-transcriptional regulatory element (WPRE); f) a nucleotide sequence comprising a 3′ LTR comprising an insulator in a forward orientation relative to the nucleotide sequence encoding the transgene of interest, wherein the insulator is on the same strand of a nucleic acid as the transgene; and g) a nucleotide sequence comprising an attB (GA) recombination site.

In one example, the provirus construct comprises, in order from 5′ to 3′: a) a nucleotide sequence comprising an attB (GT) recombination site; b) a nucleotide sequence comprising a 5′ LTR; c) lentiviral elements; d) a nucleotide sequence comprising a MND promoter operably linked to a nucleotide sequence encoding a transgene of interest; e) a nucleotide sequence comprising a woodchuck hepatitis virus post-transcriptional regulatory element (WPRE); f) a nucleotide sequence comprising a 3′ LTR comprising an insulator in a reverse orientation relative to the nucleotide sequence encoding the transgene of interest, wherein the insulator is on the same strand of a nucleic acid as the transgene; and g) a nucleotide sequence comprising an attB (GA) recombination site.

In one example, the lentiviral elements are selected from the group consisting of a nucleotide sequence comprising psi packaging signal (v); a nucleotide sequence comprising gug and pol genes; a nucleotide sequence encoding a rev protein; a nucleotide sequence comprising a central polypurine tract (cPPT) and combinations thereof.

In one example, lentiviral element is a nucleotide sequence comprising psi packaging signal (v). In one example, lentiviral element is a nucleotide sequence comprising gag and pol genes. In one example, lentiviral element is a nucleotide sequence encoding a rev protein. In one example, lentiviral element is a nucleotide sequence comprising a central polypurine tract (cPPT).

In one example, the provirus construct comprises, in order from 5′ to 3′: a) a nucleotide sequence comprising an attB (GT) recombination site; b) a nucleotide sequence comprising a 5′ LTR; c) lentiviral elements; d) a nucleotide sequence comprising a MND promoter operably linked to a nucleotide sequence encoding a mScarlet-I; e) a nucleotide sequence comprising a woodchuck hepatitis virus post-transcriptional regulatory element (WPRE): f) a nucleotide sequence comprising a 3′ LTR comprising an insulator; and g) a nucleotide sequence comprising an attB (GA) recombination site.

In one example, the provirus construct comprises, in order from 5′ to 3′: a) a nucleotide sequence comprising an attB (GT) recombination site; b) a nucleotide sequence comprising a 5′ LTR: c) lentiviral elements; d) a nucleotide sequence comprising a MND promoter operably linked to a nucleotide sequence encoding a mScarlet-I; e) a nucleotide sequence comprising a woodchuck hepatitis virus post-transcriptional regulatory element (WPRE); f) a nucleotide sequence comprising a 3′ LTR comprising an insulator in a forward orientation relative to the nucleotide sequence encoding the transgene of interest, wherein the insulator is on the same strand of a nucleic acid as the transgene; and g) a nucleotide sequence comprising an attB (GA) recombination site.

In one example, the provirus construct comprises, in order from 5′ to 3′: a) a nucleotide sequence comprising an attB (GT) recombination site; b) a nucleotide sequence comprising a 5′ LTR: c) lentiviral elements; d) a nucleotide sequence comprising a MND promoter operably linked to a nucleotide sequence encoding a mScarlet-I; e) a nucleotide sequence comprising a woodchuck hepatitis virus post-transcriptional regulatory element (WPRE): f) a nucleotide sequence comprising a 3′ LTR comprising an insulator in a reverse orientation relative to the nucleotide sequence encoding the transgene of interest, wherein the insulator is on the same strand of a nucleic acid as the transgene; and g) a nucleotide sequence comprising an attB (GA) recombination site.

In one example, the provirus construct comprises, in order from 5′ to 3′: a) a nucleotide sequence comprising an attB (GT) recombination site; b) a nucleotide sequence comprising a 5′ LTR; c) lentiviral elements; d) a nucleotide sequence comprising a MND promoter comprising a sequence set forth in SED ID NO: 39 operably linked to a nucleotide sequence encoding a transgene of interest: e) a nucleotide sequence comprising a woodchuck hepatitis virus post-transcriptional regulatory element (WPRE) comprising a sequence set forth in SEQ ID NO: 41; f) a nucleotide sequence comprising a 3′ LTR comprising an insulator comprising a sequence set forth in SEQ ID NO: 29; and g) a nucleotide sequence comprising an attB (GA) recombination site.

In one example, the provirus construct comprises, in order from 5′ to 3′: a) a nucleotide sequence comprising an attB (GT) recombination site; b) a nucleotide sequence comprising a 5′ LTR; c) lentiviral elements; d) a nucleotide sequence comprising a MND promoter comprising a sequence set forth in SED ID NO: 39 operably linked to a nucleotide sequence encoding a transgene of interest: e) a nucleotide sequence comprising a woodchuck hepatitis virus post-transcriptional regulatory element (WPRE) comprising a sequence set forth in SEQ ID NO: 41; f) a nucleotide sequence comprising a 3′ LTR comprising an insulator comprising a sequence set forth in SEQ ID NO: 30; and g) a nucleotide sequence comprising an attB (GA) recombination site.

In one example, the provirus construct comprises, in order from 5′ to 3′: a) a nucleotide sequence comprising an attB (GT) recombination site; b) a nucleotide sequence comprising a 5′ LTR; c) lentiviral elements; d) a nucleotide sequence comprising a MND promoter comprising a sequence set forth in SED ID NO: 39 operably linked to a nucleotide sequence encoding a transgene of interest: c) a nucleotide sequence comprising a woodchuck hepatitis virus post-transcriptional regulatory element (WPRE) comprising a sequence set forth in SEQ ID NO: 41; f) a nucleotide sequence comprising a 3′ LTR comprising an insulator comprising a sequence set forth in SEQ ID NO: 31; and g) a nucleotide sequence comprising an attB (GA) recombination site.

In one example, the provirus construct of the disclosure is integrated into a vector (i.e., to generate the provirus vector of the disclosure). In one example, the vector is a plasmid or a virus. In one example, the provirus vector has a pUC57 backbone.

The present disclosure provides a provirus vector comprising the provirus construct of the disclosure.

In one example, the provirus vector comprises: the provirus construct as described herein and additionally a) a nucleotide sequence encoding a suicide marker operably linked to a nucleotide sequence comprising a promoter; and b) a nucleotide sequence comprising a polyA signal.

In one example, the provirus vector comprises, in order from 5′ to 3′, a) the provirus construct as described herein, b) a nucleotide sequence encoding a suicide marker operably linked to a nucleotide sequence comprising a promoter; and c) a nucleotide sequence comprising a polyA signal

In one example, the provirus vector comprises, in order from 5′ to 3′: a) the provirus construct as described herein; b) a nucleotide sequence encoding a suicide marker operably linked to a nucleotide sequence comprising a promoter, wherein the promoter is a CMV promoter, a CMV enhancer, a MND promoter, a SV40 promoter with enhancer, a UBC promoter, a PGK promoter, a EFIA promoter, a hACTB promoter or a CAG promoter; and c) a nucleotide sequence comprising a polyA signal, wherein the polyA signal is a SV40 polyA. SVLP polyA, hGH polyA, BGH poly A or rbGlob poly A.

In one example, the provirus vector comprises, in order from 5′ to 3′: a) the provirus construct as described herein: b) a nucleotide sequence comprising a CMV promoter operably linked to a nucleotide sequence encoding a Herpes Simplex Virus-1 thymidine kinase (HSV-TK) suicide marker; and c) a nucleotide sequence comprising a SV40 polyA signal.

In one example, the provirus vector comprises, in order from 5′ to 3′: a) the provirus construct as described herein; b) a nucleotide sequence comprising a CMV promoter operably linked to a nucleotide sequence encoding a Herpes Simplex Virus-1 thymidine kinase (HSV-TK) suicide marker; and c) a nucleotide sequence comprising a SV40 polyA signal.

In one example, the provirus vector comprises, in order from 5′ to 3′: a) the provirus construct as described herein; b) a nucleotide sequence comprising a CMV promoter operably linked to a nucleotide sequence encoding a Herpes Simplex Virus-1 thymidine kinase (HSV-TK) suicide marker comprising a sequence set forth in SEQ ID NO: 6; and c) a nucleotide sequence comprising a SV40 polyA signal.

In one example, the provirus vector comprises, in order from 5′ to 3′: a) the provirus construct as described herein; b) a nucleotide sequence comprising a CMV promoter operably linked to a nucleotide sequence encoding a Herpes Simplex Virus-1 thymidine kinase (HSV-TK) suicide marker comprising a sequence set forth in SEQ ID NO: 6; and c) a nucleotide sequence comprising a SV40 polyA signal.

In one example, the provirus vector comprises, in order from 5′ to 3′: a) the provirus construct as described herein; b) a nucleotide sequence comprising a CMV promoter operably linked to a nucleotide sequence encoding a Herpes Simplex Virus-1 thymidine kinase (HSV-TK) suicide marker comprising a sequence set forth in SEQ ID NO: 6; and c) a nucleotide sequence comprising a SV40 polyA signal.

In one example, the provirus vector comprises or consists of a sequence set forth in SEQ ID NO: 48. In one example, the provirus vector comprises a sequence set forth in SEQ ID NO: 48. In one example, the provirus vector consists of a sequence set forth in SEQ ID NO: 48.

The present disclosure also provides a method of stably integrating the provirus construct described herein into the stable cell line described herein (i.e., the cell line comprising the landing pad cassette of the disclosure), wherein the provirus construct is integrated between the first and a second site-specific recombination sites present in the stable cell line. For example, the first and a second site-specific recombination sites present in the stable cell line are attP. In one example, integration of the provirus construct excises the landing pad plasmid sequences.

In one example, the method comprises transfecting the provirus construct into the cell line in a cell culture in the presence of a recombinase. Exemplary recombinases suitable for use in the present disclosure will be apparent to the skilled person and/or described herein, and include, for example, Bxb1. Cre, BciVI (Bful), Bcul (SpeI), EcoRI, Aatll, AgeI (BshTI), Apal, BamHI, BgIII, Blpl (Bpu1102I), BsrGI (Bsp1407), Clal (Bsu15I), EcoRI, EcoRV (Eco32I), Eam1104I (EarI), Hindlll, Kpnl, Mlul, Ncol, Ndel, Nhcl, Notl, Nsil, Pstl, Pvul, Pvull, SacL SalI, ScaI, SpeI, XbaI, Xhol, Sacll (Cfr421) and XbaI.

In one example, the recombinase is a serine recombinase. In one example, the serine recombinase is Bxb1. In another example, the serine recombinase is human Bxb1.

In one example, the sequence encoding the recombinase is codon optimized. For example, the sequence encoding the human Bxb1 recombinase is codon optimized.

In one example, the recombinase and provirus vector are added to the cell culture at a ratio of 5 to 1. In one example, the recombinase and provirus vector are added to the cell culture at a ratio of 4 to 1. In one example, the recombinase and provirus vector are added to the cell culture at a ratio of 3 to 1. In one example, the recombinase and provirus vector are added to the cell culture at a ratio of 2 to 1. In one example, the recombinase and provirus vector are added to the cell culture at a ratio of 1 to 1.

In one example, the cell is a mammalian cell. For example, the cell is a Jurkat cell, a HEK293 cell, a HEK293T cell, a HEK293T/17 cell, a GPRG cell, a GPRTG cell, a K562 cell, a U-937 cell, a CHO cell, a hematopoietic progenitor or stem cell (e.g. CD34+ cell), a monocyte, a macrophage, a peripheral blood mononuclear cell, a hepatocyte, a CD4+T lymphocyte, a CD8+T lymphocyte, a dendritic cell, or a derivative thereof.

In one example, the method comprises selecting the cells comprising the integrated provirus construct. For example, selecting the cells comprising the integrated provirus construct comprises adding a compound to activate the suicide marker in the cells that do not have the integrated provirus construct. In one example, the compound is ganciclovir (GCV). GCV elaidic acid ester, penciclovir, acyclovir, valacyclovir, (E)-5-(2-bromovinyl)-2′-deoxyuridine, zidovudine or 2-Exo-methanocarbathymidine. In one example, the compound is ganciclovir (GCV). In one example, the compound is GCV elaidic acid ester. In one example, the compound is penciclovir, acyclovir. In one example, the compound is valacyclovir. In one example, the compound is (E)-5-(2-bromovinyl)-2-deoxyuridine. In one example, the compound is zidovudine. In one example, the compound is 2-Exo-methanocarbathymidine.

In one example, the compound is added to the cell culture medium at a concentration of at least 0.1 ng/ml of cell culture medium. In one example, the compound is added at a concentration of between about 0.001 μg/mL and about 100,000 μg/mL of cell culture medium. In one example, the compound is added at a concentration of between about 0.01 μg/mL and about 100,000 μg/mL of cell culture medium. In one example, the compound is added at a concentration of between about 0.01 μg/mL and about 10,000 μg/mL of cell culture medium. In one example, the compound is added at a concentration of between about 0.1 μg/mL and about 100,000 μg/mL of cell culture medium. In one example, the compound is added at a concentration of between about 0.1 μg/mL and about 10,000 μg/mL of cell culture medium. In one example, the compound is added at a concentration of between about 0.1 μg/ml, and about 1,000 μg/mL of cell culture medium. In one example, the compound is added at a concentration of between about 0.1 μg/mL and about 100 μg/mL of cell culture medium. In one example, the compound is added at a concentration of between about 0.1 μg/mL and about 10 μg/mL of cell culture medium. For example, the compound is added at a concentration of about 0.1 μg/mL, or about 0.2 μg/mL, or about 0.3 μg/mL, or about 0.4 μg/mL, or about 0.5 μg/mL, or about 0.6 μg/mL, or about 0.7 μg/mL, or about 0.8 μg/mL, or about 0.9 μg/mL, or about 1 μg/mL of cell culture medium. In one example, the compound is added at a concentration of about 1 μg/mL of cell culture medium.

In one example, the compound is added to the cell culture medium for a sufficient period of time to result in cell death of cells not expressing the provirus construct. In one example, the compound is added for a period of between 1 and 30 days. For example, the compound is added for a period of between 5 and 20 days. In one example, the compound is added for a period of about 6 days, or about 8 days, or about 10 days, or about 12 days, or about 13 days, or about 14 days, or about 15 days, or about 16 days, or about 18 days, or about 20 days. In one example, the compound is added for a period of about 14 days.

In one example, the compound is added at a concentration of about 1 μg/mL of cell culture medium for a period of about 14 days.

In one example, the compound is added to the cell culture medium from day 0, or day 1, or day 2, or day 3, or day 3, or day 4, day 5, day 6, day 7, day 8 or day 9 of the culture period. For example, the compound is added to the cell culture medium from day 0 (i.e., the start of the culture period). In another example, the compound is added to the cell culture medium from day 1 of the culture period. In a further example, the compound is added to the cell culture medium from day 2 of the culture period. In one example, the compound is added to the cell culture medium from day 3 of the culture period. In another example, the compound is added to the cell culture medium from day 4 of the culture period. In a further example, the compound is added to the cell culture medium from day 5 of the culture period. In one example, the compound is added to the cell culture medium from day 6 of the culture period. In one example.

the compound is added to the cell culture medium from day 7 of the culture period. In another example, the compound is added to the cell culture medium from day 8 of the culture period. In a further example, the compound is added to the cell culture medium from day 9 of the culture period.

In one example, the cells comprising the provirus construct are expanded to produce a stable cell line. For example, the present disclosure comprises a stable cell line comprising the provirus construct as described herein.

In one example, the expanded cells are used to determine the efficacy of the provirus construct. In another example, the expanded cells are used to determine the efficacy of the components of the provirus construct. For example, the components of the provirus construct comprise the insulator, the promoter and/or the enhancer and combinations thereof.

In one example, the expanded cells are used to determine the efficacy of an insulator in a given provirus. In one example, the expanded cells are used to determine the efficacy of an insulator in a forward orientation relative to a nucleotide sequence encoding the transgene of interest, wherein the insulator is on the same strand of a nucleic acid as the transgene. In one example, the expanded cells are used to determine the efficacy of an insulator in a reverse orientation relative to a nucleotide sequence encoding the transgene of interest, wherein the insulator is on the same strand of a nucleic acid as the transgene. For example, in assessing the efficacy of an insulator in a given provirus, the insulator may reduce expression of LMO2 mRNA relative to cells without an insulator. In another example, in assessing the efficacy of an insulator in a given provirus, the insulator in a forward orientation may reduce expression of LMO2 mRNA relative to cells without an insulator. In another example, in assessing the efficacy of an insulator in a given provirus, the insulator in a reverse orientation may reduce expression of LMO2 mRNA relative to cells without an insulator. In other examples assessing the efficacy of an insulator in a given provirus, the insulator may not reduce or docs not detectably reduce or does not significantly reduce expression of LMO2 mRNA relative to cells without the insulator. In one example, in assessing the efficacy of an insulator in a given provirus, the insulator in a forward orientation relative to a nucleotide sequence encoding the transgene of interest may not reduce or does not detectably reduce or does not significantly reduce expression of LMO2 mRNA relative to cells without the insulator. In another example, in assessing the efficacy of an insulator in a given provirus, the insulator in a reverse orientation relative to a nucleotide sequence encoding the transgene of interest may not reduce or does not detectably reduce or does not significantly reduce expression of LMO2 mRNA relative to cells without the insulator.

For example, the insulator reduces expression of LMO2 mRNA by at least 10%, or at least 15%, or at least 20%, or at least 25%, or at least 30%, or at least 35%, or at least 40%, or at least 45%, or at least 50%, or at least 55%, or at least 60%, or at least 65%, or at least 70%, or at least 75%, or at least 80%, or at least 85%, or at least 90% relative to cells without the insulator.

For example, an insulator that does not detectably reduce expression of LMO2 RNA may reduce the level of expression at a level that is less than the limit of detection of the assay being used to detect the expression.

For example, an insulator that does not significantly reduce expression of LMO2 mRNA reduces expression by no more than 5% or 4% or 3% or 2% or 1% relative to cells without the insulator.

In one example, the insulator in a forward orientation relative to a nucleotide sequence encoding the transgene of interest reduces expression of LMO2 mRNA by at least 10% relative to cells without the insulator. In one example, the insulator in a forward orientation relative to a nucleotide sequence encoding the transgene of interest reduces expression of LMO2 mRNA by at least 15% relative to cells without the insulator. In one example, the insulator in a forward orientation relative to a nucleotide sequence encoding the transgene of interest reduces expression of LMO2 mRNA by at least 20% relative to cells without the insulator. In one example, the insulator in a forward orientation relative to a nucleotide sequence encoding the transgene of interest reduces expression of LMO2 mRNA by at least 25% relative to cells without the insulator. In one example, the insulator in a forward orientation relative to a nucleotide sequence encoding the transgene of interest reduces expression of LMO2 mRNA by at least 30% relative to cells without the insulator. In one example, the insulator in a forward orientation relative to a nucleotide sequence encoding the transgene of interest reduces expression of LMO2 mRNA by at least 35% relative to cells without the insulator. In one example, the insulator in a forward orientation relative to a nucleotide sequence encoding the transgene of interest reduces expression of LMO2 mRNA by at least 40% relative to cells without the insulator. In one example, the insulator in a forward orientation relative to a nucleotide sequence encoding the transgene of interest reduces expression of LMO2 mRNA by at least 45% relative to cells without the insulator. In one example, the insulator in a forward orientation relative to a nucleotide sequence encoding the transgene of interest reduces expression of LMO2 mRNA by at least 50% relative to cells without the insulator. In one example, the insulator in a forward orientation relative to a nucleotide sequence encoding the transgene of interest reduces expression of LMO2 mRNA by at least 55% relative to cells without the insulator. In one example, the insulator in a forward orientation relative to a nucleotide sequence encoding the transgene of interest reduces expression of LMO2 mRNA by at least 60% relative to cells without the insulator. In one example, the insulator in a forward orientation relative to a nucleotide sequence encoding the transgene of interest reduces expression of LMO2 mRNA by at least 65% relative to cells without the insulator. In one example, the insulator in a forward orientation relative to a nucleotide sequence encoding the transgene of interest reduces expression of LMO2 mRNA by at least 70% relative to cells without the insulator. In one example, the insulator in a forward orientation relative to a nucleotide sequence encoding the transgene of interest reduces expression of LMO2 mRNA by at least 75% relative to cells without the insulator. In one example, the insulator in a forward orientation relative to a nucleotide sequence encoding the transgene of interest reduces expression of LMO2 mRNA by at least 80% relative to cells without the insulator. In one example, the insulator in a forward orientation relative to a nucleotide sequence encoding the transgene of interest reduces expression of LMO2 mRNA by at least 85% relative to cells without the insulator. In one example, the insulator in a forward orientation relative to a nucleotide sequence encoding the transgene of interest reduces expression of LMO2 mRNA by at least 90% relative to cells without the insulator.

In one example, the insulator in a reverse orientation relative to a nucleotide sequence encoding the transgene of interest reduces expression of LMO2 mRNA by at least 10% relative to cells without the insulator. In one example, the insulator in a reverse orientation relative to a nucleotide sequence encoding the transgene of interest reduces expression of LMO2 mRNA by at least 15% relative to cells without the insulator. In one example, the insulator in a reverse orientation relative to a nucleotide sequence encoding the transgene of interest reduces expression of LMO2 mRNA by at least 20% relative to cells without the insulator. In one example, the insulator in a reverse orientation relative to a nucleotide sequence encoding the transgene of interest reduces expression of LMO2 mRNA by at least 25% relative to cells without the insulator. In one example, the insulator in a reverse orientation relative to a nucleotide sequence encoding the transgene of interest reduces expression of LMO2 mRNA by at least 30% relative to cells without the insulator. In one example, the insulator in a reverse orientation relative to a nucleotide sequence encoding the transgene of interest reduces expression of LMO2 mRNA by at least 35% relative to cells without the insulator. In one example, the insulator in a reverse orientation relative to a nucleotide sequence encoding the transgene of interest reduces expression of LMO2 mRNA by at least 40% relative to cells without the insulator. In one example, the insulator in a reverse orientation relative to a nucleotide sequence encoding the transgene of interest reduces expression of LMO2 mRNA by at least 45% relative to cells without the insulator. In one example, the insulator in a reverse orientation relative to a nucleotide sequence encoding the transgene of interest reduces expression of LMO2 mRNA by at least 50% relative to cells without the insulator. In one example, the insulator in a reverse orientation relative to a nucleotide sequence encoding the transgene of interest reduces expression of LMO2 mRNA by at least 55% relative to cells without the insulator. In one example, the insulator in a reverse orientation relative to a nucleotide sequence encoding the transgene of interest reduces expression of LMO2 mRNA by at least 60% relative to cells without the insulator. In one example, the insulator in a reverse orientation relative to a nucleotide sequence encoding the transgene of interest reduces expression of LMO2 mRNA by at least 65% relative to cells without the insulator. In one example, the insulator in a reverse orientation relative to a nucleotide sequence encoding the transgene of interest reduces expression of LMO2 mRNA by at least 70% relative to cells without the insulator. In one example, the insulator in a reverse orientation relative to a nucleotide sequence encoding the transgene of interest reduces expression of LMO2 mRNA by at least 75% relative to cells without the insulator. In one example, the insulator in a reverse orientation relative to a nucleotide sequence encoding the transgene of interest reduces expression of LMO2 mRNA by at least 80% relative to cells without the insulator. In one example, the insulator in a reverse orientation relative to a nucleotide sequence encoding the transgene of interest reduces expression of LMO2 mRNA by at least 85% relative to cells without the insulator. In one example, the insulator in a reverse orientation relative to a nucleotide sequence encoding the transgene of interest reduces expression of LMO2 mRNA by at least 90% relative to cells without the insulator.

In one example, the expanded cells are used to determine the efficacy of a promoter. For example, the expanded cells are used to determine the level of leakage of the promoter, or level of unwanted transcription of the promoter. In one example, the promoter increases expression of mRNA relative to cells without the promoter. In one example, the promoter increases expression of mRNA relative to cells without the promoter and without unwanted transcription.

In one example, the expanded cells are used to determine the efficacy of an enhancer. For example, the enhancer increases expression of mRNA relative to cells without the enhancer.

The present disclosure also provides the use of the stable cell line comprising the integrated provirus construct for production of an enveloped virus, wherein the virus is for use in gene therapy.

In one example, the enveloped virus is a retrovirus. For example, the enveloped virus is a lentivirus, e.g., human immunodeficiency virus.

The present disclosure provides a method of assessing components of a provirus construct. For example, the present disclosure provides a method of assessing the efficacy of components of a provirus construct. In one example, the disclosure provides a method for screening components of a provirus construct.

The present disclosure also provides a method of developing a cell line for screening modified components of a provirus construct. For example, the disclosure provides an improved cell line for use as a screening tool for modified components of a provirus construct.

In one example, the cell line is used to identify effective provirus construct components.

In one example, the provirus construct components are regulatory elements. For example, the provirus construct component is an insulator. For example, the provirus construct component is an insulator in a forward orientation relative to a nucleotide sequence encoding the transgene of interest, wherein the insulator is on the same strand of a nucleic acid as the transgene. In example, the provirus construct component is an insulator in a reverse orientation relative to a nucleotide sequence encoding the transgene of interest, wherein the insulator is on the same strand of a nucleic acid as the transgene. In another example, the provirus construct component is a promoter. In a further example, the provirus construct component is an enhancer. In yet a further example, the provirus construct component is a lentiviral element.

The present disclosure also provides a method of optimising a provirus construct, comprising integrating a provirus construct into a cell line comprising the landing pad cassette of the disclosure, and assessing the activity of the integrated provirus construct.

In one example, the activity of the integrated provirus construct is determined by measuring gene expression relative to cells without the integrated provirus construct.

The present disclosure also provides a method for assessing safety, genotoxicity and/or efficacy of a provirus construct or modified provirus construct component, comprising: a) integrating the provirus construct into a stable cell line comprising a landing pad cassette of the disclosure integrated at a specific locus: b) selecting the cells comprising the integrated provirus construct, wherein selecting the cells comprising the integrated provirus construct comprises adding a compound to activate the suicide marker in the cells that do not have the integrated provirus construct; c) expanding the cells comprising the provirus construct to produce a stable cell line; and d) measuring the expression of the locus to determine the efficacy of the provirus construct or modified provirus construct component.

The present disclosure also provides a method for assessing safety, genotoxicity and/or efficacy of a provirus construct provirus construct or modified provirus construct component, comprising: a) integrating the landing pad plasmid as described herein into the genome of a cell at a specific locus; b) selecting the cells comprising the landing pad plasmid, wherein selecting comprises selecting the cells expressing the detectable marker by fluorescence-activated cell sorting and/or selecting the cells resistant to antibiotic treatment by addition of an antibiotic; c) expanding the cells comprising the landing pad plasmid to produce a stable cell line; d) integrating the provirus construct into the stable cell line of step c), wherein the provirus construct is integrated between the first and the second site-specific recombination sites present in the stable cell line; e) selecting the cells comprising the integrated provirus construct, wherein selecting the cells comprising the integrated provirus construct comprises adding a compound to activate the suicide marker in the cells that do not have the integrated provirus construct; f) expanding the cells comprising the provirus construct to produce a stable cell line; and g) measuring the expression of the locus to determine the safety, genotoxicity and/or efficacy of the provirus construct or modified provirus construct component.

The present disclosure also provides a method for assessing safety, genotoxicity and/or efficacy of a provirus construct or modified provirus construct component, comprising: a) integrating the landing pad plasmid as described herein into the genome of a cell at a specific locus; b) selecting the cells comprising the landing pad plasmid, wherein selecting comprises selecting the cells expressing the detectable marker by fluorescence-activated cell sorting and/or selecting the cells resistant to antibiotic treatment by addition of an antibiotic; c) expanding the cells comprising the landing pad plasmid to produce a stable cell line; d) integrating the provirus construct into the stable cell line of step c), wherein the provirus construct is integrated between the first and the second site-specific recombination sites present in the stable cell line; e) selecting the cells comprising the integrated provirus construct, wherein selecting the cells comprising the integrated provirus construct comprises adding a compound to activate the suicide marker in the cells that do not have the integrated provirus construct; f) selecting single cells comprising the provirus construct and expanding the single clones; and g) measuring the expression of the locus to determine the safety, genotoxicity and/or efficacy of the provirus construct or modified provirus construct component. In one example, the modified provirus construct component is a regulatory element. For example, the regulatory element is an insulator, a promoter, an enhancer, a lentiviral element or combinations thereof. In one example, the regulatory element is an insulator. In one example, the regulatory element is an insulator in a forward orientation relative to a nucleotide sequence encoding the transgene of interest, wherein the regulatory element is on the same strand of a nucleic acid as the transgene. In one example, the regulatory element is an insulator in a reverse orientation relative to a nucleotide sequence encoding the transgene of interest, wherein the regulatory element is on the same strand of a nucleic acid as the transgene. In one example, the regulatory element is a promoter. In one example, the regulatory element is a lentiviral element.

In one example, the provirus construct is a construct as described herein.

In one example, the method further comprises comparing the safety, genotoxicity and/or efficacy of at least two provirus or modified provirus construct components to identify an optimal provirus or modified provirus construct component.

In one example, safety, genotoxicity and/or efficacy of the modified regulatory element is determined by measuring expression of the gene locus relative to cells without the modified regulatory element. Methods of determining expression of the gene locus will be apparent to the skilled person and/or described herein. For example, efficacy of the modified regulatory element is determined by measuring mRNA levels of the gene locus in cells with the modified regulatory element compared to mRNA levels of the gene locus in cells without the modified regulatory element. For example, LMO2 mRNA levels in cells with the modified regulatory element are determined and compared to LMO2 mRNA levels in cells without the modified regulatory element.

### BRIEF DESCRIPTION OF THE DRAWINGS

FIG. 1 is a schematic representation of (A) a first, (B) a second, (C) a third, (D) a fourth, (E) a fifth, and (F) a sixth exemplary landing pad cassette of the disclosure.

FIG. 2 is a schematic representation of (A) a first, (B) a second, (C) a third, (D) a fourth, (E) a fifth, and (F) a sixth exemplary landing pad plasmid of the disclosure.

FIG. 3 is a schematic representation of an exemplary provirus construct of the disclosure.

FIG. 4 is a schematic representation of (A) a first and (B) a second exemplary landing pad vector map of the disclosure.

FIG. 5 is a schematic representation of an exemplary provirus vector map.

FIG. 6 is a schematic representation of landing pad integration and clonal assay and bulk assay.

FIG. 7 is a schematic representation of the experimental workflow of the clonal assay and bulk assay.

FIG. 8 is a series of graphical representations showing (A) mean fluorescent intensity (selected clones shown in rectangular boxes); (B) vector copy number; and (C) recombination efficiency by droplet digital PCR (ddPCR) following provirus integration into landing pad comprising Jurkat cells.

FIG. 9 is a graphical representation showing (A) GCV selection and (B) LMO2 mRNA expression in Jurkat cells transfected with plasmids of the disclosure. Data are normalized to K562 cells.

FIG. 10 is a graphical representation showing GCV treatment selects against landing pad clones.

FIG. 11 is a graphical representation showing LMO2 mRNA levels in sorted mScarlet+ cells with positive control: MoMLV gammaretrovirus; and negative control: EF1a short promoter. Data are normalized to insulator-free construct.

FIG. 12 is a graphical representation showing a comparison between (A) mean fluorescent intensity in a single cell clonal assay; and (B) mean fluorescent intensity in the bulk cell assay of the disclosure.

FIG. 13 is a graphical representation showing RT-qPCR comparison of bulk cells. 5 sorted cells (from bulk cell selection using single cell clone analysis) and single cells clones.

FIG. 14 is a graphical representation showing LMO2 mRNA levels expression in Jurkat cells transfected with plasmids of the disclosure in a single clone assay. Data are normalized to K562 cells.

FIG. 15 is a graphical representation showing (A) percentage LMO2 mRNA 10 expression in Jurkat cells transfected with plasmids of the disclosure; and (B) the data in triplicate. Data are normalized to insulator-free construct.

### Key to Sequence Listing

SEQ ID NO: 1 Nucleotide sequence of Exemplary Landing Pad Plasmid No. 1
SEQ ID NO: 2 Nucleotide sequence of Exemplary Landing Pad Plasmid No. 2
SEQ ID NO: 3 Nucleotide sequence of CMV enhancer
SEQ ID NO: 4 Nucleotide sequence of CMV promoter
SEQ ID NO: 5 Nucleotide sequence of MND promoter
SEQ ID NO: 6 Nucleotide sequence of Herpes Simplex Virus-1 thymidine kinase (HSV-TK)
SEQ ID NO: 7 Nucleotide sequence of enhanced Green Fluorescent Protein (eGFP)
SEQ ID NO: 8 Nucleotide sequence of puromycin
SEQ ID NO: 9 Amino acid sequence of P2A
SEQ ID NO: 10 Nucleotide sequence of P2A 1
SEQ ID NO: 11 Nucleotide sequence of P2A 2
SEQ ID NO: 12 Nucleotide sequence of P2A 3
SEQ ID NO: 13 Nucleotide sequence of P2A 4
SEQ ID NO: 14 Amino acid sequence of T2A
SEQ ID NO: 15 Nucleotide sequence of T2A 1
SEQ ID NO: 16 Nucleotide sequence of T2A 2
SEQ ID NO: 17 Nucleotide sequence of SV40 promoter with enhancer
SEQ ID NO: 18 Nucleotide sequence of SV40 polyA
SEQ ID NO: 19 Nucleotide sequence of polyA
SEQ ID NO: 20 Nucleotide sequence of woodchuck hepatitis virus post-transcriptional regulatory element (WPRE)
SEQ ID NO: 21 Nucleotide sequence of LMO2_gRNA5
SEQ ID NO: 22 Nucleotide sequence of LMO2_g1
SEQ ID NO: 23 Nucleotide sequence of LMO2_g2
SEQ ID NO: 24 Nucleotide sequence of 5′ homology arm for HDR (LMO2 intron 1)
SEQ ID NO: 25 Nucleotide sequence of 3′ homology arm for HDR (LMO2 intron 1)
SEQ ID NO: 26 Nucleotide sequence of 5′ homology arm for HDR (LMO2 promoter)
SEQ ID NO: 27 Nucleotide sequence of 3′ homology arm for HDR (LMO2 promoter)
SEQ ID NO: 28 Nucleotide sequence of chicken hypersensitive site-4 (cHS4) insulator_650 bp_insulator (3SA) (forward)
SEQ ID NO: 29 Nucleotide sequence of cHS4_650 bp_insulator (forward)
SEQ ID NO: 30 Nucleotide sequence of cHS4_400 bp_insulator (reverse)
SEQ ID NO: 31 Nucleotide sequence of cHS4_250 bp_core_insulator (reverse)
SEQ ID NO: 32 Nucleotide sequence of Insulator 22-3 (forward)
SEQ ID NO: 33 Nucleotide sequence of Insulator A1 (forward)
SEQ ID NO: 34 Nucleotide sequence of Insulator A2 (forward)
SEQ ID NO: 35 Nucleotide sequence of Foamy virus insulator (FV) (forward)
SEQ ID NO: 36 Nucleotide sequence of Insulator FB (forward)
SEQ ID NO: 37 Nucleotide sequence of Insulator SNS5 (forward)
SEQ ID NO: 38 Nucleotide sequence of cHS4 insulator_1200 bp_full sequence (forward)
SEQ ID NO: 39 Nucleotide sequence of MND promoter
SEQ ID NO: 40 Nucleotide sequence of mScarlet-I
SEQ ID NO: 41 Nucleotide sequence of WPRE (mut6)
SEQ ID NO: 42 Nucleotide sequence of RRE
SEQ ID NO: 43 Nucleotide sequence of cPPT
SEQ ID NO: 44 Nucleotide sequence of R
SEQ ID NO: 45 Nucleotide sequence of U5
SEQ ID NO: 46 Nucleotide sequence of U3
SEQ ID NO: 47 Nucleotide sequence of gag
SEQ ID NO: 48 Nucleotide sequence of Exemplary Provirus No. 1
SEQ ID NO: 49 Nucleotide sequence of B4 (forward)
SEQ ID NO: 50 Nucleotide sequence of C1 (forward)
SEQ ID NO: 51 Nucleotide sequence of shrna743
SEQ ID NO: 52 Nucleotide sequence of shrna743 alternative with no poly T tail
SEQ ID NO: 53 Nucleotide sequence of 7SK promoter
SEQ ID NO: 54 Nucleotide sequence of 7SK promoter alternative
SEQ ID NO: 55 Nucleotide sequence of BGH poly A
SEQ ID NO: 56 Nucleotide sequence of chicken hypersensitive site-4 (cHS4) insulator_650 bp_insulator (3SA) (reverse)
SEQ ID NO: 57 Nucleotide sequence of cHS4_650 bp_insulator (reverse)
SEQ ID NO: 58 Nucleotide sequence of cHS4_400 bp_insulator (forward)
SEQ ID NO: 59 Nucleotide sequence of cHS4_250 bp_core_insulator (forward)
SEQ ID NO: 60 Nucleotide sequence of Insulator 22-3 (reverse)
SEQ ID NO: 61 Nucleotide sequence of Insulator A1 (reverse)
SEQ ID NO: 62 Nucleotide sequence of Insulator A2 (reverse)
SEQ ID NO: 63 Nucleotide sequence of Foamy virus insulator (FV) (reverse)
SEQ ID NO: 64 Nucleotide sequence of Insulator FB (reverse)
SEQ ID NO: 65 Nucleotide sequence of Insulator SNS5 (reverse)
SEQ ID NO: 66 Nucleotide sequence of cHS4 insulator_1200 bp_full sequence (reverse)
SEQ ID NO: 67 Nucleotide sequence of B4 (reverse)
SEQ ID NO: 68 Nucleotide sequence of C1 (reverse)
SEQ ID NO: 69 Nucleotide sequence of alternative cHS4_400 bp insulator (forward)
SEQ ID NO: 70 Nucleotide sequence of alternative cHS4_400 bp insulator (reverse)

### DETAILED DESCRIPTION

### General

Throughout this specification, unless specifically stated otherwise or the context requires otherwise, reference to a single step, composition of matter, group of steps or group of compositions of matter shall be taken to encompass one and a plurality (i.e., one or more) of those steps, compositions of matter, groups of steps or groups of compositions of matter.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the present disclosure.

Any example of the present disclosure herein shall be taken to apply mutatis mutandis to any other example of the disclosure unless specifically stated otherwise.

Unless specifically defined otherwise, all technical and scientific terms used herein shall be taken to have the same meaning as commonly understood by one of ordinary skill in the art (for example, in immunology, immunohistochemistry, protein chemistry, and biochemistry).

Unless otherwise indicated, the recombinant protein, cell culture, and immunological techniques utilised in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al. Molecular Cloning: A Laboratory Manual, Cold Spring Harbour Laboratory Press (1989). T. A. Brown (editor). Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology. Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Selected Definitions

As used herein, the term “nucleotide sequence” or “nucleic acid sequence” will be understood to mean a series of contiguous nucleotides (or bases) covalently linked to a phosphodiester backbone. By convention, sequences are presented from the 5′ end to the 3′ end, unless otherwise specified. As used herein, the term “5′” indicates the end of the molecule known by convention as the “upstream” end, and the term “3′” indicates the end of the molecule known by convention as the “downstream” end.

As used herein, the term “polynucleotide” refers a molecular chain of nucleotides chemically bonded by a series of ester linkages between the phosphoryl group of one nucleotide and the hydroxyl group of the sugar in an adjacent nucleotide.

As used herein, the term “isolated” will be understood to mean a naturally occurring nucleic acid sequence. DNA fragment. DNA molecule, coding sequence, or oligonucleotide that is removed from its natural environment, or is a synthetic molecule or cloned product.

The term “recombinant” shall be understood to mean the product of artificial genetic recombination.

As used herein, the term “encode”, “encodes” or “encoding” refers to a region of a polynucleotide capable of undergoing translation into a polypeptide.

The term “polypeptide” or “polypeptide chain” will be understood to mean a series of contiguous amino acids linked by peptide bonds. For example, a protein shall be taken to include a single polypeptide chain i.e., a series of contiguous amino acids linked by peptide bonds or a series of polypeptide chains covalently or non-covalently linked to one another (i.e., a polypeptide complex). The series of polypeptide chains can be covalently linked using a suitable chemical or a disulfide bond. Examples of non-covalent bonds include hydrogen bonds, ionic bonds, Van der Waals forces, and hydrophobic interactions.

As used herein, the term “cassette” will be understood to include a polynucleotide sequence encoding a polypeptide to be expressed and sequences controlling its expression such as a promoter.

As used herein, the term “vector” refers to a small DNA molecule that carries foreign DNA into another host cell. It will be apparent to the skilled person that the term vector encompasses plasmids, as well as other naturally occurring or artificially synthesised vectors.

As used herein, the term “plasmid” will be understood to mean a small, circular, double-stranded DNA molecule which is capable of replicating independently and facilitating expression of a nucleic acid in a host cell.

As used herein, the term “provirus” will be understood to refer to the genetic material of a virus as incorporated into, and able to replicate with, the genome of a host cell.

As used herein, the term “operably linked to” means positioning a promoter (or other regulatory element) relative to a nucleic acid sequence such that expression of the nucleic acid is controlled or regulated by the promoter.

As used herein, the term “select” or “selecting” or “selection” will be understood to refer to sorting of a cell or population of cells having a marker of interest, such as by positive or negative selection. For example, the cells expressing the marker of interest can be selected for by positive selection (i.e., selecting cells expressing the marker) or by negative selection (i.e., excluding cells expressing the marker). As described in more detail below, the marker is selected from the group consisting of a suicide marker, a detectable marker and a selection marker.

### Polynucleotides

The present disclosure provides an isolated polynucleotide comprising a nucleotide sequence encoding a promoter operably linked to a nucleotide sequence encoding a detectable marker; and a nucleotide sequence encoding a suicide marker. The present disclosure also provides a landing pad cassette comprising the polynucleotide of the disclosure.

The polynucleotide of the present disclosure includes DNA and RNA (e.g., mRNA).

In one example, the polynucleotide is a DNA (e.g., DNA vector). In one example, the polynucleotide is a RNA (e.g., RNA vector).

Polynucleotides of the disclosure can be isolated or produced using any method known in the art, for example, amplification (e.g., using PCR or splice overlap extension).

For example, nucleic acid (e.g., genomic DNA or RNA that is then reverse transcribed to form cDNA) is used as a template in a polymerase chain reaction (PCR) to amplify a polynucleotide of the disclosure. Methods of PCR are known in the art and described, for example, in Dieffenbach (ed) and Dveksler (ed), 1995. Generally, for PCR two non-complementary nucleic acid primer molecules comprising at least about 20 nucleotides in length, and more preferably at least 25 nucleotides in length are hybridized to different strands of a nucleic acid template, and specific nucleic acid copies of the template are amplified enzymatically. Preferably, the primers hybridize to nucleic acid adjacent to a polynucleotide of the disclosure, thereby facilitating amplification of the entire nucleic acid.

Other methods for the production of a polynucleotide and cassette of the disclosure will be apparent to the skilled artisan, for example, in Ausubel et al 1987. Sambrook et al, 2001.

The resulting polynucleotide can then be introduced into the landing pad plasmid of the disclosure using a standard method in the art (such as those described in Sambrook et al, 2001). Methods for cloning a polynucleotide into a plasmid of the disclosure will be apparent to the skilled artisan.

### Landing Pads

The present disclosure provides a landing pad plasmid comprising the landing pad cassette of the disclosure.

### Landing Pad Cassette

The present disclosure provides a landing pad cassette comprising a polynucleotide of the disclosure.

The term “landing pad cassette” as used herein refers to a polynucleotide construct capable of site-specific chromosomal DNA integration into mammalian cells. It will be apparent to the skilled person from the disclosure herein that the landing pad cassette comprises one or more promoters operably linked with a nucleic acid encoding a polypeptide (e.g., a detectable marker).

In one example, the landing pad cassette contains the nucleotide sequence encoding a marker (e.g., a suicide marker) used for selection.

In one example, the landing pad cassette is adapted at the 5′ and 3′ ends to facilitate insertion into a plasmid (i.e., a landing pad plasmid). For example, the landing pad cassette comprises restriction endonuclease site at the 5′ and 3′ ends. The skilled person will appreciate that such restriction sites allow for the insertion of the landing pad cassette into a plasmid without disrupting the remainder of the DNA.

In one example, the landing pad cassette comprises site-specific recombination sites to facilitate provirus integration. For example, the site-specific recombination site(s) are attP recombination sites. In another example, the site-specific recombination site(s) are attB recombination sites.

### Landing Pad Plasmid

The present disclosure further provides a landing pad plasmid comprising the landing pad cassette of the disclosure.

The term “landing pad plasmid” is to be taken in its broadest context and includes a nucleic acid comprising one or more promoters operably linked with a nucleic acid encoding a polypeptide.

In one example, the landing pad plasmid has a pcDNA3.1 (+) backbone.

In one example, the landing pad cassette is positionally and sequentially oriented within the plasmid such that the polynucleotide in the cassette can be transcribed into RNA, and when necessary, translated into a protein or a polypeptide, undergo appropriate post-translational modifications required for activity in the transformed cell, and be translocated to the appropriate compartment for biological activity by targeting to appropriate intracellular compartments or secretion into extracellular · compartments.

### Homology Arms

In one example, the landing pad plasmid of the disclosure comprises a nucleotide sequence comprising a 5′ homology arm (HA) and a nucleotide sequence comprising a 3′ HA.

As used herein, the term “homology arms” will be understood to be guide sequences, which are designed to enable the specific alignment to a specific locus within the genome and then, through homologous recombination, replacement of the polynucleotide sequence between the homology arms. The homology arms are referred to herein as 5′ homology arm and a 3′ homology arm.

In one example, the homology arms have a length ranging from 500 base pairs to several kilobases of DNA on both sides of the polynucleotide or landing pad cassette. For example, the homology arms have a length about 600 base pairs of DNA. In one example, the homology arms have a length about 750 base pairs of DNA.

### 5′Untranslated Region (5′-UTR)

As used herein, the term “5′-untranslated region” or “5′-UTR” refers to a non-coding region of an mRNA located at the 5′end of the translation initiation sequence (AUG). The 5′-UTR is upstream from the coding sequence. Within the 5′-UTR is a sequence that is recognized by a ribosome which allows the ribosome to bind and initiate translation.

For example, 5′-UTRs include 5′-UTR of haptoglobin (HP), human immunodeficiency virus 1 (HIV-1), simian immunodeficiency virus (SIV), fibrinogen beta chain (FGB), haptoglobin-related protein (HPR), albumin (ALB), complement component 3 (C3), fibrinogen alpha chain (FGA), alpha 6 collagen (Col6A), alpha-1-antitrypsin (SERPINA1), alpha-1-antichymotrypsin (SERPINA3) a fragment and/or a variant thereof.

### 3′Untranslated Region (3′-UTR)

As used herein, the term “3′-untranslated region” or “3′-UTR” refers to a region of an mRNA located at the 3′end of the translation termination codon (i.e., stop codon). The 3′-UTR is found immediately following a translation stop codon. The 3′-UTR plays a critical role in translation termination as well as post-transcriptional modification.

For example, 3′-UTRs include a 3′-UTR of arachidonate 5-lipoxygenase (ALOX5), human immunodeficiency virus 1 (HIV-1), simian immunodeficiency virus (SIV), alpha I collagen (COL1A1), tyrosine hydroxylase (TH) gene, amino-terminal enhancer of split (AES), human mitochondrial 12S rRNA (mtRNR1), a fragment and/or a variant thereof.

### Provirus

The present disclosure provides a provirus vector comprising the provirus construct of the disclosure. In one example, the provirus vector has a pUC57 backbone.

### Provirus Construct

The present disclosure provides a provirus construct comprising a nucleotide sequence encoding a 5′ long terminal repeat (LTR) comprising an insulator; a nucleotide sequence encoding a transgene of interest operably linked to a promoter; and a nucleotide sequence encoding a 3′ LTR comprising the insulator.

### Long Terminal Repeat

The provirus construct of the present disclosure comprises a nucleotide sequence encoding a 5′ long terminal repeat (LTR) comprising an insulator; and a nucleotide sequence encoding a 3′ LTR comprising the insulator.

As used herein, “long terminal repeat” or “LTR” shall be understood to refer to a sequence that is typically at least several hundred bases long, usually bearing inverted repeats at its termini (often starting with TGAA and ending with TTCA), and flanked with short direct repeats duplicated within the cell DNA sequences flanking an insertion site. The short inverted repeats are involved in integrating the full length viral, retrotransposon, or vector DNA into the host genome. The integration sequence is sometimes called att, for attachment. Inside the LTRs reside three distinct subregions: U3 (the enhancer and promoter region, transcribed from the 5′-LTR), R (repeated at both ends of the RNA), and U5 (transcribed from the 5′-LTR). The LTR and its associated flanking sequences (primer binding sites, splice sites, dimerization linkage and encapsidation sequences) comprise the cis-acting sequences of a retroviral vector. Sources of LTR nucleic acid sequences, e.g., nucleic acid fragments or segments, include, but are not limited to murine retroviruses, murine VL30 sequences, retrotransposons, simian retroviruses, avian retroviruses, feline retroviruses, lentiviruses, avian retroviruses and bovine retroviruses, foamy viruses.

In one example, the nucleotide sequence comprising the R comprises or consists of a sequence set forth in SEQ ID NO: 44. In one example, the nucleotide sequence comprising the R comprises of a sequence set forth in SEQ ID NO: 44. In one example, the nucleotide sequence comprising the R consists of a sequence set forth in SEQ ID NO: 44.

In one example, the nucleotide sequence comprising the U5 comprises or consists of a sequence set forth in SEQ ID NO: 45. In one example, the nucleotide sequence comprising the U5 comprises of a sequence set forth in SEQ ID NO: 45. In one example, the nucleotide sequence comprising the U5 consists of a sequence set forth in SEQ ID NO: 45.

In one example, the nucleotide sequence comprising the U3 comprises or consists of a sequence set forth in SEQ ID NO: 46. In one example, the nucleotide sequence comprising the U3 comprises of a sequence set forth in SEQ ID NO: 46. In one example, the nucleotide sequence comprising the U3 consists of a sequence set forth in SEQ ID NO: 46.

LTRs contain regulatory elements, such as insulators, promoters and/or enhancers. In one example, the LTR is modified to reduce or increase the effectiveness of these regulatory elements. As used herein “effectiveness” can be used to refer to increased transcription, or suppression of transcription. In another example, the insulator is modified to increase the function thereof. For example, modification of the insulator may comprise changing the orientation of the insulator relative to a nucleotide sequence encoding the transgene of interest. In one example, the promoter is modified to increase the function thereof. In one example, the enhancer is modified to increase the function thereof. In other examples, regulatory elements are introduced into a LTR. For example, modified insulators, promoters and/or enhancers can be introduced into a LTR.

The nature of the retroviral replication process is that the U3 region of both the 5′ and 3′ LTRs of the provirus construct are effectively copied from the 3′ LTR of the provirus construct in the preceding generation. Thus, it will be understood that after one round of replication, any changes in the 3′LTR will be copied into 5′ LTR.

### Insulators

The provirus construct of the present disclosure comprises a nucleotide sequence encoding a 5′ long terminal repeat (LTR) comprising an insulator; and a nucleotide sequence encoding a 3′ LTR comprising the insulator.

The term “insulators” as used herein refers to a genetic boundary element that blocks the interaction between enhancers and promoters. Insulators are an exogenous DNA sequence that can be added to prevent, upon integration of the construct into the genome of a host cell, nearby genomic sequences from influencing expression of the integrated polynucleotides, and prevent the integrated construct from influencing the expression of nearby genomic sequences.

By residing between the enhancer and promoter, the insulator may inhibit their subsequent interactions. Insulators can determine the set of genes an enhancer can influence.

Insulators shield genes from inappropriate cis-regulatory signals, e.g., enhancer elements. Insulator activity is thought to occur primarily through the 3D structure of DNA mediated by proteins including CCCTC-binding factor (CTCF). CTCF is the main insulator-binding protein in vertebrates and also provides chromatin barrier functions. Barrier insulators may prevent the spread of heterochromatin from a silenced gene to an actively transcribed gene. Insulators may also provide increased expression levels of a transgene in a construct of interest.

In one example, the insulator is duplicated upon integration of the construct into the host cell genome, such that the insulator flanks the integrated construct (e.g., within the LTR region) and acts to insulate the integrated polynucleotide sequences.

In one example, the insulator is in a forward orientation relative to a nucleotide sequence encoding the transgene of interest. In one example, the insulator is in a reverse orientation relative to a nucleotide sequence encoding the transgene of interest.

As used herein, “forward orientation” shall be understood to refer to the sequence of a regulatory element (e.g., an insulator), or the like, running in a 5′ to 3′ direction relative to a nucleotide sequence encoding the transgene of interest which is on the same strand of a nucleic acid as the regulatory element. Accordingly, “reverse orientation” shall be understood to refer to the complementary strand of the sequence of the regulatory element, or the like, running in a 3′ to 5′ direction relative to a nucleotide sequence encoding the transgene of interest which is on the same strand of a nucleic acid as the regulatory element.

In one example, the orientation of the insulator relative to a nucleotide sequence encoding the transgene of interest does not affect expression of the transgene of interest. For example, the insulator in a forward orientation relative to a nucleotide sequence encoding the transgene of interest reduces expression of the transgene of interest. In another example, the insulator in a reverse orientation relative to a nucleotide sequence encoding the transgene of interest reduces expression of the transgene of interest.

The insulators include insulators from a α-globin locus, for example, chicken HS4, or from a β-globin locus (see Chung et aL, 1993. Cell 74:505; Chung et aL, 1997. PNAS 94:575; Bell et aL, 1999. Cell 98:387; and PCT/US2015/020369, incorporated by reference herein).

### Transgenes of Interest

In some examples, the provirus construct comprises a nucleotide sequence encoding a transgene of interest. In some examples, the transgene of interest may be any transgene. In other examples, the transgene of interest may be selected from an exemplary transgene described herein. In some examples, the transgene will depend on the specific use for which the provirus is intended. Exemplary transgenes include a transgene coding for a therapeutic RNA (e.g., encoding an antisense complementary RNA of a target RNA or DNA sequence), a transgene encoding for a protein that is deficient or absent in a subject affected with a pathology, or a transgene used for vaccination with DNA, i.e., a transgene coding for a protein, the expression of which will induce vaccination of the recipient body against said protein. In some examples, the transgene encodes a protein or nucleic acid useful for treating a hemoglobinopathy, e.g., sickle cell disease or a thalassemia. In some examples, the transgene encodes a protein or nucleic acid useful for treating a primary immunodeficiency (PID) or a rare genetic disorder. For example, the PID or rare genetic disorder may include Activated PI3K Delta Syndrome (APDS), X-linked Agammaglobulinemia (XLA), Ataxia Telangiectasia. Chronic Granulomatous Disease (CGD) and Other Phagocytic Cell Disorders, Common Variable Immune Deficiency (CVID), Complement Deficiencies, DiGeorge Syndrome, Hemophagocytic Lymphohistiocytosis (HLH). Hyper IgE Syndrome. Hyper IgM Syndromes. IgG Subclass Deficiency, Innate Immune Defects. Toll-like Receptor (TLR) Deficiencies (including MyD88 Deficiency, IRAK4 Deficiency, UNC93B Deficiency and TLR3 Mutations), Human Natural Killer Cell Deficiencies. Defects in Interferon-γ (IFN-γ) and Interleukin-12 (IL-12) Signaling, Leukocyte Adhesion Deficiency (LAD), NEMO Deficiency Syndrome. Selective IgA Deficiency. Selective IgM Deficiency. Severe Combined Immune Deficiency (SCID) and Combined Immune Deficiency, Specific Antibody Deficiency, Transient Hypogammaglobulinemia of Infancy, WHIM Syndrome (Warts, Hypogammaglobulinemia. Infections, and Myelokathexis). Wiskott-Aldrich Syndrome. Antibody Deficiency with Normal or Elevated Immunoglobulins, Immunodeficiency with Thymoma (Good's Syndrome), Transcobalamin II Deficiency, Kappa Chain Deficiency, Heavy Chain Deficiencies, Post-Meiotic Segregation (PMS2) Disorder, Unspecified Hypogammaglobulinemia, Chronic Mucocutaneous Candidiasis (CMC), Cartilage Hair Hypoplasia (CHH). X-linked Lymphoproliferative (XLP) Syndromes 1 and 2. X-linked Immune Dysregulation with Polyendocrinopathy (IPEX) Syndrome, Veno-occlusive Disease (VODI), Hoyeraal-Hreidarsson Syndrome (Dyskeratosis Congenita), Immunodeficiency with Centromeric Instability and Facial Anomalies (ICF), Schimke Syndrome, Comel-Netherton Syndrome, Diamond Blackfan Anemia, Schwachmann Diamond Syndrome, Deficiency of Adenosine Deaminase 2. Krabbe Disease (GLD). Alpha-mannosidosis. Nijmegen Breakage Syndrome, graft-versus-host disease (GvHD), deficiency of adenosine deaminase 2 (ADA2), deficiency of interleukin-1-receptor antagonist (IL-1RA), scleroderma, homozygous familial hypercholesterolemia (HoFH), systemic lupus erythematosus (SLE), or chronic inflammatory demyelinating polyneuropathy (CIDP). In some examples, the transgene encodes a protein or nucleic acid useful for treating Wiskott-Aldrich Syndrome (WAS).

### Provirus Vector

The present disclosure provides a provirus vector comprising a provirus construct of the disclosure.

In one example, the provirus vector is a plasmid or a virus. In one example, the vector has a pUC57 backbone.

In one example, the provirus vector further comprises lentiviral elements. As used herein, the term “lentiviral element” refers to any viral component present in lentiviruses. For example, the lentiviral elements are selected from the group consisting of: a) a nucleotide sequence comprising a psi packaging signal (w); b) a nucleotide sequence comprising gag and pol genes; c) a nucleotide sequence encoding a rev protein; d) a nucleotide sequence comprising a central polypurine tract (cPPT), and combinations thereof. In one example, the gug and pol genes are human codon optimized. In one example, the nucleotide sequence comprising gag and pol genes lacks a functional rev responsive element (RRE). In one example, the pol gene encodes an inactive integrase enzyme.

In one example, the nucleotide sequence comprising the gag gene comprises or consists of a sequence set forth in SEQ ID NO: 47. In one example, the nucleotide sequence comprising the gag gene comprises of a sequence set forth in SEQ ID NO: 47. In one example, the nucleotide sequence comprising the gag gene consists of a sequence set forth in SEQ ID NO: 47.

In one example, the nucleotide sequence comprising the RRE comprises or consists of a sequence set forth in SEQ ID NO: 42. In one example, the nucleotide sequence comprising the RRE comprises of a sequence set forth in SEQ ID NO: 42. In one example, the nucleotide sequence comprising the RRE consists of a sequence set forth in SEQ ID NO: 42.

In one example, the nucleotide sequence comprising the cPPT comprises or consists of a sequence set forth in SEQ ID NO: 43. In one example, the nucleotide sequence comprising the cPPT comprises of a sequence set forth in SEQ ID NO: 43. In one example, the nucleotide sequence comprising the cPPT consists of a sequence set forth in SEQ ID NO: 43.

### Additional Elements of the Landing Pads and Proviruses of the Disclosure

### Suicide Markers

The present disclosure provides an isolated polynucleotide, a landing pad cassette, a landing pad plasmid and a provirus vector comprising a suicide marker.

As used herein, the term “suicide marker” is used herein to define any gene that is involved in the process of cell death or apoptosis. A suicide marker, inserted into a target cell, provides a way to sensitive cells to certain chemotherapeutic agents or prodrugs. For example, the suicide marker can be pharmacologically activated by chemotherapeutic agents or prodrugs to eliminate cells expressing the suicide marker. In some examples, the suicide marker encodes a protein capable of converting a drug precursor into a cytotoxic compound thereby killing the cell. A suicide marker can be incorporated into a polynucleotide, cassette or plasmid to reduce the risk of direct toxicity, uncontrolled proliferation, or for use as a selection or isolation tool. For example, the suicide marker will allow for selection of cells which do not have the suicide marker. In another example, wherein the suicide marker is present in the landing pad plasmid, the suicide marker will allow for selection of cells with unwanted random integration events.

Exemplary suicide markers are known in the art and/or described herein. For example, the suicide marker can be thymidine kinase (e.g., HSK-TK), caspase-9, caspase-8, cytosine deaminase, uracil phosphoribosyl transferase, nucleoside purine phosphorylase or thymidylate kinase.

Exemplary chemotherapeutic agents or prodrugs for activating the suicide markers are known in the art and/or described herein. For example, the drug may be ganciclovir (GCV); GCV elaidic acid ester, penciclovir, acyclovir, valacyclovir, (E)-5-(2-bromovinyl)-2′-deoxyuridine, zidovudine, 2′-Exo-methanocarbathymidine.

Examples of suicide genes and corresponding chemotherapeutic agents or prodrugs are disclosed in the table below.

TABLE 1
Suicide genes and corresponding agents

| Suicide gene | prodrug |
|---|---|
| Thymidine Kinase | Ganciclovir; Ganciclovir elaidic acid ester; |
| | penciclovir; Acyclovir; Valacyclovir; (E)-5-(2- |
| | bromovinyl)-2′-deoxyuridine; zidovudine; 2′- |
| | Exo-methanocarbathymidine |
| Cytosine deaminase | 5-Fluorocytosine |
| Purine nucleoside | 6-Methylpurine deoxyriboside; |
| phosphorylase | Fludarabine |
| uracil phosphoribosyl | 5-Fluorocytosine; 5-Fluorouracil |
| transferase | |
| thymidylate kinase. | Azidothymidine |

For example, the suicide marker is the Herpes simplex virus thymidine kinase type 1 (HSV-TK) and the prodrug is gancyclovir (GCV). In this example, the HSV-TK phosphorylates nucleoside analogs (gancyclovirlacyciovir) into their monophosphate and triphosphate forms, which are incorporated into DNA during cell division, leading to cell death.

### Detectable Markers

The present disclosure provides an isolated polynucleotide, a landing pad cassette, a landing pad plasmid and a provirus vector comprising a detectable marker.

As used herein, the term “detectable marker” is used herein to define any gene that expresses a detectable protein, including, but not limited to, radioactive isotopes, fluorescent proteins, chemiluminescers, chromophores, enzymes, enzyme substrates, enzyme cofactors, enzyme inhibitors, semiconductor nanoparticles, dyes, metal ions, metal sols, ligands (e.g., biotin, streptavidin or haptens) and the like. An example of a detectable marker is one that encodes a non-functional gene product but that is still detectable by fluorescence means. In some examples, a detectable marker is included so that the cell that expresses the polynucleotide is identifiable, for example for qualitative, quantitative and/or selection purposes. The detectable marker may be detectable by any suitable means in the art, including by flow cytometry, fluorescence, spectophotometry, and so forth. For example, the detectable marker will allow for visualisation of cells containing the detectable marker.

Several fluorescent genes are known in the art and include, for example, those that encode green fluorescent protein (GFP), enhanced green fluorescent protein (EGFP), a yellow fluorescent protein (YFP), such as mBanana, a red fluorescent protein (RFP), such as mCherry, DsRed, dTomato, tdTomato, mHoneydew, or mStrawberry, TagRFP, far-red fluorescent pamidronate (FRFP), such as mGrape1 or mGrape2, a cyan fluorescent protein (CFP), a blue fluorescent protein (BFP), enhanced cyan fluorescent protein (ECFP), ultramarine fluorescent protein (UMFP), orange fluorescent protein (OFP), such as mOrange or mTangerine, red (orange) fluorescent protein (mROFP), TagCFP, or a tetracystein fluorescent motif. These proteins permit selection of a cell expressing the detectable marker protein using standard techniques as known in the art and described herein, e.g., fluorescence activated cell sorting (FACS).

In other examples, the detectable marker expresses an intracellular or cell-surface marker that can be labelled by immunofluorescence staining. For example, the cell-surface marker is labelled by a fluorescently-labelled specific binding protein for the cell-surface marker. In another example, the detectable marker is an enzyme that catalyses a detectable reaction. In one example, the enzymatic reporter genes include β-galactosidase, alkaline phosphatase, firefly luciferase or Renilla luciferase. For example, the expression of β-galactosidase is detected by the addition of the substrate 5-bromo-4-chloro-3-indolyl-beta-D-galactopyranoside (x-gal), which is hydrolyzed by β-galactosidase to produce a blue colored precipitate. Alternatively, the expression of either firefly luciferase or Renilla luciferase is detected by addition of a substrate that in the presence of the relevant protein is luminescent and is detectable, for example, using a spectrophotometer.

### Selection Markers

The present disclosure provides an isolated polynucleotide, a landing pad cassette, a landing pad plasmid and a provirus vector comprising a selection marker.

As used herein, the term “selection marker” or “selectable marker gene” is used herein to define any gene that confers resistance to a toxic compound, for example antibiotic resistance. If such a selectable marker is expressed by a cell, consequently such a cell or a population of such cells can be selected for. For example, the selection marker will allow for selection of cells with the selection marker. In one example, the selection marker promotes inhibition or death of the cells comprising the marker.

Examples of selection marker genes conferring resistance to antibiotics include neomycin resistance gene (neo), hygromycin resistance gene (php), puromycin N-acetyl-transferase (pac), histidinol dehydrogenase (hisD), zeocin resistance gene (zco), blemycin resistance gene (ble) and blasticidin S deaminase (bsd).

Exemplary compounds, drugs or antibiotics include G418, hygromycin B, puromycin, zeocin, blasticidin S and L-histidinol.

Examples of selection marker genes and corresponding compounds, drugs or antibiotics are disclosed in the table below.

TABLE 2
Selection genes and corresponding agents

| Selection gene | Drug | Short name |
|---|---|---|
| Neomycin resistance gene | G418 | neo |
| Hygromycin resistance gene | Hygromycin B | hph |
| puromycin N-acetyl-transferase | Puromycin | pac |
| Zeocin/Bleomycin resistance gene | Zeocin | zeo/ble |
| Blasticidin S deaminase | Blasticidin S | bsd |
| Histidinol dehydrogenase | L-Histidinol | hisD |

### Promoters

As used herein, the term “promoter” is to be taken in its broadest context and refers to synthetic or naturally-derived molecule which is capable of conferring, activating or enhancing expression of a nucleic acid in a cell. A promoter may comprise one or more specific transcriptional regulatory sequences to further enhance expression and/or to alter the spatial expression and/or temporal expression of a nucleic acid in a cell. A promoter may also comprise distal enhancer or repressor elements, which may be located as much as several thousand base pairs, or anywhere in the genome, from the start site of transcription. A promoter may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, hormones, toxins, drugs, pathogens, metal ions, or inducing agents.

A promoter may be “endogenous” or “exogenous” or “heterologous.” An “endogenous” promoter is one which is naturally linked with a given gene in the genome. An “exogenous” or “heterologous” promoter is one which is placed in juxtaposition to a gene by means of genetic manipulation (i.e., molecular biological techniques) such that transcription of that gene is directed by the linked promoter.

Examples of promoters include a cytomegalovirus (CMV) promoter, a CMV enhancer, a MND promoter, a simian virus 40 (SV40) promoter with enhancer, a UBC promoter, a PGK promoter, a EF1A promoter, a hACTB promoter, a CAG promoter or a bla promoter.

### Linkers

As described herein, the components of the polynucleotides, landing pad cassette, landing pad plasmid and/or provirus vector are directly or indirectly linked to each other.

In some example, the components of the polynucleotides, landing pad cassette and/or landing pad plasmid are indirectly linked, e.g., via a linker.

In some examples, the linker is an IRES element or encodes a 2A self-cleaving peptide. As used herein, the term “internal ribosome entry site” or “IRES” refers to a sequence of nucleotides within a mRNA to which a ribosome or a component thereof, e.g., a 40S subunit of a ribosome, is capable of binding. An IRES need not necessarily comprise nucleic acid that induces translation of a mRNA (e.g., a start codon: AUG). An IRES suitable for use in the present disclosure will be apparent to the skilled person and/or are described herein.

In one example, the IRES is derived from encephalomyocarditis virus (EMCV). For example, the IRES is a wild-type IRES from EMCV.

In one example, the IRES is derived from a fibroblast growth factor 1A (FGF1A) IRES.

In addition, synthetic IRES elements have been described, which can be designed, according to methods know in the art to mimic the function of naturally occurring IRES elements (see Chappell, S A et al. Proc. Natl Acad. Sci. USA (2000) 97 (4): 1536-41).

As used herein, the term “2A self-cleaving peptide” refers to a peptide that possesses autoproteolytic activity and is capable of cleaving itself from a larger polypeptide moiety. Non-limiting examples of 2A self-cleaving peptides suitable for the compositions and methods of the present disclosure include the peptide sequences from porcine teschovirus-1 2A (P2A), a foot-and-mouth disease virus (FMDV) 2A (F2A), an Equine Rhinitis A Virus (ERAV) 2A (E2A), a Thosea asigna virus 2A (T2A), a cytoplasmic polyhedrosis virus 2 A (BmCPV2A), a Flacherie Virus 2A (BmIFV2A), or a combination thereof.

### Polyadenylation Signal

As used herein, the term “polyadenylation signal” or “polyA signal” refers to a nucleotide sequence which directs both termination and polyadenylation. Polyadenylation is typically understood to be the addition of a polyA sequence to a polynucleotide. The polyadenylation signal may be located within a nucleotide sequence at the 3′-end of the polynucleotide to be polyadenylated. The polyA signal can be “heterologous” or “endogenous”. An endogenous poly A signal is one that is found naturally at the 3′ end of the coding region of a given gene in the genome. A heterologous polyA signal is one which is one which is isolated from one gene and placed 3′ of another gene.

Suitable polyA signal for use in the present disclosure will be apparent to the skilled person and/or are described herein. For example, the polyA signal can include a SV40 poly A. SVLP polyA, hGH polyA, BGH polyA or rbGlob polyA.

### Woodchuck Hepatitis Virus Post-Transcriptional Regulatory Element (WPRE)

In one example, the polynucleotide, landing pad cassette, landing pad plasmid and/or provirus vector of the disclosure further comprises a nucleotide sequence comprising a woodchuck hepatitis virus post-transcriptional regulatory element (WPRE). For example, the WPRE is located 3′ of the nucleotide sequence encoding the suicide marker.

As used herein, the term “woodchuck hepatitis virus post-transcriptional regulatory element” or “WPRE” refers to a DNA sequence that, when transcribed, creates a tertiary structure enhancing expression. Insertion of a WPRE element can stimulate expression of detectable markers.

Exemplary WPRE will be apparent to the skilled person and/or are described herein. For example, an exemplary WPRE is set forth in SEQ ID NO: 20.

### Short Hairpin RNA

In one example, the polynucleotide, landing pad cassette, landing pad plasmid and/or provirus vector of the disclosure further comprises a nucleotide sequence comprising a short hairpin RNA (shRNA). For example, the shRNA is located 3′ of the nucleotide sequence encoding the promoter. In one example, the shRNA is a shrna743. In another example, the shRNA is a shrna743 alternative. In one example, the promoter is a 7SK promoter.

As used herein, the term “short hairpin RNA” or “shRNA” refers to a RNA sequence that can silence target gene expression.

Exemplary shRNA will be apparent to the skilled person and/or are described herein. For example, an exemplary shRNA is set forth in SEQ ID NO: 51 and 52.

### Recombination Sites

As used herein, the term “recombination sites” or “site-specific recombination sites” refers to specific polynucleotide sequences that are recognized by the recombinase enzymes described herein. Typically, two different sites are involved (termed “complementary sites”), one present in the target nucleic acid (e.g., a chromosome or episome of a eukaryote) and another on the nucleic acid that is to be integrated at the target recombination site. To facilitate a clear description of the recombination sites, particular sites are referred to as e.g., a “first recombination site” and a “second recombination site”. It is to be understood that the first and second sites can appear in any desired order or orientation, unless otherwise specified, and that no particular order or orientation is intended by the words “first”, “second” etc.

In one example, the site-specific recombination site(s) are attP recombination sites. In another example, the site-specific recombination site(s) are attB recombination sites.

As used herein, the terms “attB” and “attP,” refer to attachment (or recombination) sites originally from a bacterial target and a phage donor, respectively. The skilled person will understand that recombination sites typically include left and right arms separated by a core or spacer region. Thus, an attB recombination site consists of BOB′, where B and B′ are the left and right arms, respectively, and O is the core region. Similarly, attP is POP′, where P and P′ are the arms and O is the core region. Upon recombination between the attB and attP sites, and concomitant integration of a nucleic acid at the target, the recombination sites that flank the integrated DNA are referred to as “attL” and “attR.” The attL and attR sites, using the terminology above, thus consist of BOP′ and POB′, respectively. In some examples herein, the “O” is omitted and attB and attP, for example, are designated as BB′ and PP′, respectively.

For example, a recombination site for Cre recombinase is loxP which is a 34 base pair sequence comprising two 13 base pair inverted repeats (serving as the recombinase binding sites) flanking an 8 base pair core sequence (Sauer, B., Current Opinion in Biotechnology 5:521-527 (1994)). Other examples of loxP sites include, but are not limited to: lox511 (Hoess et aL, 1996; Bethke and Sauer. 1997), lox5171 (Lee and Saito. 1998), lox2272 (Lec and Saito, 1998), m2 (Langer et aL, 2002), lox71 (Albert et aL, 1995), and lox66 (Albert et al . . . 1995).

For example, a recombination site for the FLP recombinase include, but are not limited to: FRT (McLeod, et aL, 1996), F1, F2, F3 (Schlake and Bode, 1994), F4, F5 (Schlake and Bode. 1994). FRT (LE) (Senecoff et aL, 1988). FRT (RE) (Senecoff et aL, 1988).

### Origin of Replication

In one example, the landing pad plasmid and/or the provirus vector of the disclosure comprises a nucleotide sequence comprising an origin of replication sequence.

As used herein, the term “origin of replication” shall be understood to refer to a nucleic acid sequence at which replication is initiated on a chromosome, plasmid or virus. The origin of replication sequence is necessary to permit replication of the plasmid in a host cell. In general, such plasmid will contain at least one origin of replication sufficient to permit the autonomous stable replication of the plasmid in a host cell. For example, the origin of replication sequence is a pUC, pUK, pMB1, pMB1 (derivative), pBR322. CoIE1. R6K, p15A, pSC101 or F1. In one example, the origin of replication sequence is a viral origin of replication sequence.

In one example, the origin of replication sequence is a viral origin of replication sequence. For example, the viral origin of replication sequence is a pUC viral origin of replication sequence. In another example, the viral origin of replication sequence is a pUK viral origin of replication sequence.

### Production of Provirus Integrated Cell Lines

Methods of the disclosure are applicable to small-, mid- and large-scale productions. The methods are particularly useful for their ability to be scaled up for manufacturing pharmaceutical products at commercial scale.

Methods for the production of cell lines of the disclosure will be apparent to the skilled artisan and/or described, for example, in Ansorge et aL, (2010) Biochem. Eng. J. 48:362-377; Schweizer and Merten (2010) Curr. Gene Ther. 10:474-486; and Rodrigues et aL, (2011) Viral Gene Therapy. Xu. InTech. Chapter 2:15-40.

### Cell Lines

The present disclosure provides a cell (or cell line) for producing an enveloped virus, wherein the cell (or cell line) comprises a provirus construct of the disclosure.

As used herein, the terms “cell,” and “cell line,” may be used interchangeably. All of these terms also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a plasmid and/or expressing a polynucleotide encoded by the plasmids described herein. A host cell may be “transfected” or “transformed”.

In one example, the landing pad cassette of the disclosure is stably integrated into the cell line.

In one example, the cell (i.e., the cell comprising the landing pad cassette) is transfected with a provirus construct of the disclosure.

In one example, the provirus construct is stably integrated into the stable cell line (i.e., the cell line comprising the landing pad cassette). For example, the landing pad cassette is first integrated into the cell line to produce a stable cell line, following which the provirus construct is stably integrated into the cell line comprising the landing pad cassette.

In one example, the cell is a Jurkat cell, a HEK293 cell, a HEK293T cell, a HEK293T/17 cell, a GPRG cell, a GPRTG cell, a K562 cell, a U-937 cell, a CHO cell, a Sf9 cell, a Sf21 cell, a hematopoietic progenitor or stem cell (e.g. CD34+ cell), a monocyte, a macrophage, a peripheral blood mononuclear cell, a hepatocyte, a CD4+T lymphocyte, a CD8+T lymphocyte, a dendritic cell, or a derivative thereof. In another example, the cell is selected from a cell line derived from a Jurkat cell line, a HEK293 cell, a HEK293T cell, a HEK293T/17 cell, a GPRG cell, a GPRTG cell, a K562 cell, a U-937 cell, a CHO cell, a Sf9 cell or a Sf21 cell. In one example, the cell for producing an envelope virus is a GPRG cell. In another example, the cell for producing an envelope virus is a GPRTG cell.

In one example, the cell line comprises a nucleotide sequence encoding a mCherry detectable marker.

As used herein, the term “cell culture” will be understood to refer to the collective of the cell culture fluid or medium and the cultured cells or cell line.

The cells are cultivated in a medium suitable for cultivation of mammal cells and for producing an enveloped virus (i.e., a lentivirus). The cells can be cultivated in an adherent environment, e.g., while attached to a surface, or in a suspension environment, e.g., suspended in the medium. The medium may moreover be supplemented with additives known in the field such as antibiotics, serum (notably fetal calf serum, etc.) added in suitable concentrations. The medium may be supplemented with GlutaMax™, Pluronic™ F-68 (ThermoFisher), LONG® R3 IGF-I (Sigma-Aldrich), Cell Boost™ 5, and/or an anticlumping agent. The medium used may notably comprise serum or be serum-free. Culture media for mammal cells are known and include, for example, DMEM (Dulbecco's Modified Eagle's medium) medium, RPMI1640 or a mixture of various culture media, including for example DMEM/F12, or a scrum-free medium like optiMEM®, optiPRO®, optiPRO-SFM®, CD293® (ThermoFisher), TransFx™ (Cytiva), BalanCD® (Irvine), Freestyle F17® (Life Technologies), or Ex-Cell® 293 (Sigma-Aldrich).

Cells can be cultured using methods known in the art, such as those described in, for example, Goodman et aL, J. of ViroL, 92 (1): e01639-17 (2018) and Ryu et al. Blood. 111 (4): 1866-75 (2007).

### Integration Sites

In one example, the landing pad cassette is stably integrated into the genome of a cell at a specific locus. For example, the specific locus is associated with adverse events or genotoxicity. In another example, the specific locus is a safe-harbour locus, a common integration site (CIS) or other desired locus within the genome.

In one example, the specific locus is a leukemia oncogene (LMO2) locus, a MDS1 And EVI1 Complex (MECOM) locus, a cyclin D2 (CCND2) locus, a B lymphoma Mo-MLV insertion region 1 homolog (BMI1) locus or a meningioma (disrupted in balanced translocation) 1 (MN1) locus.

In one example, the landing pad cassette is stably integrated into the genome of a cell at or near a leukemia oncogene (LMO2) locus. The LMO2 gene encodes a LIM-domain only protein, a transcriptional cofactor critical for the development of hematopoietic stem cells. The LMO2 transcription start site (TSS) is located approximately 25 kb downstream from the 11p13 T-cell translocation cluster.

In one example, the landing pad cassette is stably integrated into the genome of a cell at a LMO2 locus, wherein the LMO2 locus is 33 kb upstream of the TSS or 2 kb downstream of the TSS.

In one example, the landing pad cassette is stably integrated into the genome of a cell at a MDS1 And EVIL Complex (MECOM) locus. MECOM (also known as EVI1) is a proto-oncogene located on chromosome 3 (3q26.2).

In one example, the landing pad cassette is stably integrated into the genome of a cell at a cyclin D2 (CCND2) locus. CCND2 is located on chromosome 12 (12p13.32).

In one example, the landing pad cassette is stably integrated into the genome of a cell at a B lymphoma Mo-MLV insertion region 1 homolog (BMI1) locus. BMI1 is located on chromosome 10 (10p12.2).

In one example, the landing pad cassette is stably integrated into the genome of a cell at a meningioma (disrupted in balanced translocation) 1 (MN1) locus. MN1 is located on chromosome 22 (22q12.3).

In another example, the specific locus is safe-harbour locus selected from a AAV integration site 1 (AAVS1), a HPRT locus, an albumin locus, a hROSA26 locus or a chemokine (CC motif) receptor 5 (CCR5) locus or for example, the specific locus is a common integration site. For example, the specific locus is a common integration site (CIS) selected from CARD8. NSD1, QRICH1, SAPS2, USP48, GPATCH8, FCHSD2, NPLOC4, SMARCC1, NF1. PACS1. HLA, or FBXL11.

It will be apparent to the skilled person from the disclosure herein that the landing pad cassette is stably integrated into the genome of a cell at or near one or more of the above referenced specific loci.

### Methods of Plasmid Integration

Methods of integrating cassettes of the disclosure will be apparent to the skilled person and/or described herein. For example, methods of integrating cassettes into a cell include, transient transfection of one or more cassettes of the disclosure into a cell, or by use of stable producing cells.

### Transfection

In one example, the cell line is transfected with a landing pad cassette or provirus construct of the disclosure. In one example, the cell line is a Jurkat cell line. In another example, the cell line is a K562 cell line. In another example, the cell line is a HEK293 cell line. In another example, the cell line is a GPRG cell line. In another example, the cell line is a GPRTG cell line.

As used herein, the term “transfected” or “transfect” or “transfection” will be understood to mean the uptake of exogenous or heterologous nucleic acid by a cell. An “exogenous” or “heterologous” nucleic acid will be understood to refer to a nucleic acid that is placed into a gene by means of genetic manipulation (i.e., molecular biological techniques).

A cell has been transfected by exogenous or heterologous nucleic acid when such nucleic acid has been introduced inside the cell.

The skilled person will understand that a cell has been “transformed” by exogenous or heterologous nucleic acid when the transfected nucleic acid effects a phenotypic change. The transforming nucleic acid can be integrated into chromosomal DNA making up the genome of the cell.

In a process using transiently transfected cells, any agent allowing transfection of plasmids may be used. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346:776-777 (1990)); and strontium phosphate DNA co-precipitation (Brash et aL, Mal. Cell BioL, 7:2031-2034 (1987)).

The conditions (e.g., amount of cassette/construct, ratio between the cassette/construct and the transfection agent, the type of mediu5m, etc.) and the transfection time may be adapted by one skilled in the art according to the characteristics of the produced virus and/or of the transgene introduced.

In one example, the method comprises transfecting the provirus construct into the cell line in a cell culture in the presence of a recombinase. For example, the recombinase and provirus construct are added to the cell culture at a ratio of 5 to 1. In one example, the recombinase and provirus construct are added to the cell culture at a ratio of 4 to 1. In one example, the recombinase and provirus construct are added to the cell culture at a ratio of 3 to 1. In one example, the recombinase and provirus construct are added to the cell culture at a ratio of 2 to 1. In one example, the recombinase and provirus construct are added to the cell culture at a ratio of 1 to 1. In one example, the serine recombinase is Bxb1. In another example, the serine recombinase is human Bxb1.

Other methods of integrating cassettes and constructs into cells are well known to those skilled in the art and/or are described herein.

### Homology Directed Recombination

In one example, a cassette and/or construct of the disclosure is integrated into the cell by homology directed recombination (HDR; i.e., knock-in). In one example, the landing pad cassette of the disclosure is integrated into the cell by homology directed recombination.

Means for integration of cassettes and constructs into a cell by homology directed recombination (HDR) will be apparent to the skilled person and/or are described herein and include, for example, site directed mutagenesis using zinc-finger nucleases, CRISPR/Cas9 mediated targeting. Cre-Lox. Flp-FRT and transcription activator-like effector nucleases (TALEN).

In one example, the cassette comprises one or more recombination sites (i.e., recombinase binding sites), such as, for example, a LoxP site (which is a recognition site of the P1 recombination enzyme Cre) or a frt site (which is a recognition site of the yeast recombinase flp). Methods for using such recombination sites are known in the art.

In one example, the cassette comprises a 5′ HA and a 3′ HA that are complementary to gRNA sequences. For example, the nucleotide sequence of the gRNA comprises or consists of a sequence set forth in SEQ ID NO: 21. In one example, the nucleotide sequence of the gRNA comprises of a sequence set forth in SEQ ID NO: 21. In one example, the nucleotide sequence of the gRNA consists of a sequence set forth in SEQ ID NO: 21.

In one example, the nucleotide sequence of the gRNA comprises or consists of a sequence set forth in SEQ ID NO: 22. In one example, the nucleotide sequence of the gRNA comprises of a sequence set forth in SEQ ID NO: 22. In one example, the nucleotide sequence of the gRNA consists of a sequence set forth in SEQ ID NO: 22.

In one example, the nucleotide sequence of the gRNA comprises or consists of a sequence set forth in SEQ ID NO: 23. In one example, the nucleotide sequence of the gRNA comprises of a sequence set forth in SEQ ID NO: 23. In one example, the nucleotide sequence of the gRNA consists of a sequence set forth in SEQ ID NO: 23.

Methods for using such CRISPR/Cas9-mediated integration are known in the art (such as those described in Chi et al, 2019).

In one example, the landing pad cassette of the disclosure is stably integrated into Jurkat cells at the LMO2 locus using CRISPR/Cas9 mediated targeting.

### Recombinase-Mediated Cassette Exchange

In one example, a cassette and/or construct of the disclosure is integrated into the cell by recombinase-mediated cassette exchange (RMCE). For example, the provirus construct is integrated into the cell by RMCE.

In one example, the integrated landing pad cassette (i.e., the landing pad cassette integrated into the cell) is replaced with the provirus construct by RMCE.

It will be apparent to the skilled person that RMCE permits unidirectional integration of a DNA fragment from one molecule into a pre-determined genomic locus. Methods for RCME are well known to those skilled in the art and/or described herein. The skilled person will understand that RMCE is a process in which site-specific recombinases exchange one gene cassette (i.e., the landing pad cassette) flanked by a pair of incompatible target sites for another cassette (i.e., the provirus construct) flanked by an identical pair of sites.

As used herein, the term “recombination site” refers to a sequence of DNA that binds to a restriction recombinase. Typically, the restriction site comprises a short sequence (e.g., of approximately 4-8 base pairs) recognised and cleaved by the restriction endonuclease.

Incorporation of cassettes and/or constructs into the genome of a cell and excision of the genome of the cell can be done using site-specific recombination systems. Such systems require a restriction recombinase, to catalyse the recombination event, and two recombination sites (Sadowski (1986) J. Bacterid. 165:341-347; Sadowski (1993) FASEB J. 7:760-767). For phage integration systems, these are referred to as attachment (att) sites, with an attP element from phage DNA and the attB element present in the bacterial genome. The two attachment sites can share as little sequence identity as a few base pairs. The recombinase protein binds to both att sites and catalyses a conservative and reciprocal exchange of DNA strands that results in integration of the plasmid DNA. Additional phage or host factors, such as the DNA bending protein IHF, integration host factor, may be required for an efficient reaction (Friedman (1988) Cell 55:545-554; Finkel & Johnson (1992) Mol. Microbiol. 6:3257-3265). Phage integrases, in association with other host and/or phage factors, also excise the phage genome from the bacterial genome during the lytic phase of bacteriophages growth cycle.

As used herein, the term “restriction recombinases” refers to a class of enzyme that occur naturally in bacteria and in some viruses. Restriction recombinases bind specifically to and cleave double-stranded DNA at specific sites within or adjacent to a restriction endonuclease site. Exemplary restriction recombinases include, for example. Bxb1, Cre, BciVI (Bful), Bcul (SpeI), EcoRI, Aatll, AgeI (BshTI), Apal, BamHI, BgIII, Blpl (Bpu1102I), BsrGI (Bsp1407), Clal (Bsu15I), EcoRL EcoRV (Eco321), Eam1104] (EarI), Hindlll, Kpnl, Mlul, Ncol, Ndel, Nhel, Notl, Nsil, Pstl, Pvul, Pvull, SacL SalI, ScaI, SpeI, XbaI, Xhol, Sacll (Cfr42I) and XbaI.

In one example, the recombination site is an att site and the recombinase is Bxb1 recombinase. In another example, the recombination site is a loxP site and the recombinase is Cre recombinase.

### Enrichment of Integrated Cell Lines

The present disclosure provides a method of stably integrating the landing pad cassette into a cell line.

The present disclosure also provides a method of stably integrating the provirus construct into a cell line comprising the landing pad cassette.

In one example, the method comprises enriching the cells comprising a cassette and/or construct of the disclosure. For example, the method comprises sorting and/or selecting cells to enrich for the landing pad cassette and/or provirus construct containing cells.

### Enrichment of Landing Pad Clones

In one example, the method comprises enriching cells comprising the landing pad cassette of the disclosure, wherein the landing pad comprises a detectable marker.

Selection of cells comprising the landing pad cassette can be done by screening the cells for a detectable marker e.g., a fluorescent protein. Methods of selecting cells based on expression of a detectable marker will be apparent to the skilled person and/or are described herein.

In one example, the cells are sorted by fluorescence-activated cell sorting (FACS) and cells comprising the detectable marker are positively selected for (i.e., cells that do not comprise the detectable marker are selected against), resulting in a population of cells comprising the landing pad cassette.

Detection of cells comprising the landing pad cassette can be done by screening the cells for a detectable marker e.g., a fluorescent protein. In one example, the cells can be visualised using FACS and the cells that express the detectable marker can be visualised.

In one example, the landing pad cassette comprises a nucleotide sequence encoding an eGFP and the host cell comprises a nucleotide sequence encoding a mCherry. In one example, the cells comprising the landing pad cassette can be selected for using FACS to select cells expressing eGFP. In one example, the cells not comprising the landing pad cassette can be selected against using FACS to remove cells not expressing eGFP. In one example, the cells comprising the landing pad cassette can be visualised using FACS to visual the cells expressing eGFP. In one example, the cells not comprising the landing pad cassette can be visualised using FACS to visual the cells expressing mCherry.

Selection of cells comprising the landing pad cassette can be done by screening the cells for a selection marker e.g., an antibiotic resistance gene. For example, cells expressing the antibiotic resistance gene can be selected for by the addition of an antibiotic compound. In this example, the cells that do not comprise the selection marker, e.g., antibiotic resistance, will be selected against, resulting in a cell population comprising the landing pad cassette. Screening the cells using a selection marker allows for selection of cells with an integrated and functional landing pad cassette.

### Enrichment of Provirus Integrated Cells

In one example, the method comprises enriching cells comprising the provirus construct of the disclosure.

In one example, the method comprises selecting the cells comprising the provirus construct of the disclosure, wherein the method comprises adding a compound to activate the suicide marker in the cells that do not have the integrated provirus construct. In one example, the method comprises adding a compound to activate the suicide marker in the cells where the provirus construct has not integrated between the site-specific recombination sites in the stable cell line.

Selection against cells comprising the landing pad cassette can be done by addition of a compound activating the suicide maker. For example, addition of ganciclovir (GCV), GCV claidic acid ester, penciclovir, acyclovir, valacyclovir. (E)-5-(2-bromovinyl)-2-deoxyuridine, zidovudine or 2-Exo-methanocarbathymidine. In one example, addition of GCV leads to cell death in cells comprising a landing pad comprising a nucleotide sequence encoding a HSV-TK suicide marker. For example, the HSV-TK phosphorylates the GCV into the monophosphate and triphosphate forms, which are incorporated into DNA during cell division, leading to cell death. The resulting cell population comprises the provirus construct.

Thus, the presence of a suicide marker in the cells of the present disclosure can be used to select against the cells that do not have an integrated provirus construct. In another example, the presence of a suicide marker in the cells of the present disclosure can be used to select against the cells with an incorrectly integrated provirus construct.

### Bulk Screening Assay

The skilled person will understand that typical methods of sorting and/or isolating provirus and/or landing pad cassette integrated cells have relied on single cell sorting techniques which are laborious and time consuming (see for example, Goodman et aL, J. of ViroL, 92 (1): e01639-17 (2018)). In addition, these methods cannot be readily scaled up to commercial scale as these typical methods involve manual handling steps and are time consuming which can weeks of process time.

The inventors' solution to these problems is to provide a method of bulk cell sorting which can be used to develop improved landing pad cassettes and provirus constructs for use in screening modified provirus construct components, optimising proviruses and provirus construct components and/or assessing safety, genotoxicity and/or efficacy of integrated proviruses and/or provirus construct components.

For example, a cell line is developed comprising a landing pad cassette as described herein. The landing pad cassette provides means by way of one or more markers that allow for bulk cell sorting to select against cells which to not have the integrated landing pad cassette. Said cell line can then be used to screen for modified provirus construct components, for example, insulators, promoters and/or enhancers. The provirus construct components are modified to increase safety, reduce genotoxicity and/or reduce or increase efficacy.

As used herein. “efficacy” or “effectiveness” can be used to refer to a change of function, for example, increased transcription or suppression of transcription. In one example, the component is modified to improve the function thereof. In another example, the component is modified to increase the function thereof. In one example, the component is modified to decrease the function thereof. In one example, the component is modified to increase transcription. In another example, the component is modified to supress transcription. In one example, the component is modified to enhance efficacy thereof.

As used herein, “enhanced efficacy” refers to an increased therapeutic effect or clinical benefit. In another example, the component is modified to improve safety thereof.

As used herein, “improved safety” refers to a reduction in adverse events or undesired outcomes, for example aberrant splice events or clonal growth advantage. In one example, the component is modified to reduce the genotoxicity thereof.

As used herein, “genotoxicity” refers to damage of the genetic information within a cell causing mutations.

In one example, the cell line comprising the landing pad cassette can be used to optimise the modified components of the provirus construct. For example, the cell line comprising the landing pad cassette can be used to optimise the modified provirus construct components such that expression is increased. In one example, the cell line comprising the landing pad cassette can be used to optimise the modified provirus construct components such that expression of the transgene of interest is increased. In another example, the cell line comprising the landing pad cassette can be used to optimise the modified provirus construct components such that expression is decreased. In one example, the cell line comprising the landing pad cassette can be used to optimise the modified provirus construct components such that expression of the transgene of interest is decreased. In another example, the cell line comprising the landing pad cassette can be used to assess the safety of a provirus construct. In one example, the cell line comprising the landing pad cassette can be used to optimise a provirus construct. For example, the cell line comprising the landing pad cassette can be used to optimise a modified provirus construct component such that unwanted transactivation is reduced and/or prevented. In another example, the cell line comprising the landing pad cassette can be used to optimise a modified provirus construct component such that expression is increased. In another example, the cell line comprising the landing pad cassette can be used to optimise a modified provirus construct component such that expression of the transgene of interest is increased.

The cell line can be used to confirm the function of modified components within the provirus construct. For example, a cell line comprising the provirus construct comprising insulator components can be used to assess insulator efficacy. In one example, the insulator reduces expression of the gene locus mRNA relative to cells without the insulator. In onc example, an insulator in a forward orientation relative to a nucleotide sequence encoding a transgene of interest reduces expression of the gene locus mRNA relative to cells without the insulator. In one example, an insulator in a reverse orientation relative to a nucleotide sequence encoding a transgene of interest reduces expression of the gene locus mRNA relative to cells without the insulator. In one example, the insulator does not reduce expression of gene locus mRNA relative to cells without the insulator. In one example, an insulator in a forward orientation relative to a nucleotide sequence encoding a transgene of interest does not reduce expression of gene locus mRNA relative to cells without the insulator. In one example, an insulator in a reverse orientation relative to a nucleotide sequence encoding a transgene of interest does not reduce expression of gene locus mRNA relative to cells without the insulator. In one example, the insulator sequence exhibits effective enhancer-blocking function such that expression of the gene locus mRNA is lower than expression in an uninsulated provirus construct. In one example, an insulator in a forward orientation relative to a nucleotide sequence encoding a transgene of interest exhibits effective enhancer-blocking function such that expression of the gene locus mRNA is lower than expression in an uninsulated provirus construct. In one example, an insulator in a reverse orientation relative to a nucleotide sequence encoding a transgene of interest exhibits effective enhancer-blocking function such that expression of the gene locus mRNA is lower than expression in an uninsulated provirus construct. In another example, the insulator exhibits reduced or no enhancer-blocking function, such that expression of the gene locus mRNA is comparable to an uninsulated construct, or at least higher than an alternative effective insulator sequence. In another example, an insulator in a forward orientation relative to a nucleotide sequence encoding a transgene of interest exhibits reduced or no enhancer-blocking function, such that expression of the gene locus mRNA is comparable to an uninsulated construct, or at least higher than an alternative effective insulator sequence. In another example, an insulator in a forward orientation relative to a nucleotide sequence encoding a transgene of interest exhibits reduced or no enhancer-blocking function, such that expression of the gene locus mRNA is comparable to an uninsulated construct, or at least higher than an alternative effective insulator sequence.

Also contemplated are methods of assessing promoters, enhancers and other lentiviral elements as known in the art and described herein.

Methods of determining expression and thus enhancer blocking activity of insulators and other components will be apparent to the skilled person and/or described herein, for example RT-qPCR.

In another example, the cell line can be used to assess the safety of a provirus construct and/or modified provirus construct component. For example, the cell line can be used determine if the integrated provirus construct causes unwanted expression. For example, expression of the gene locus may be the result of an ineffective insulator. In another example, low expression of the gene locus may be the result of an ineffective promoter.

### Validation Assays

Suitable methods for assessing the safety, genotoxicity and/or efficacy and of integration of a provirus construct or modified provirus construct component of the disclosure and/or a bulk screening assay of the disclosure are available to those skilled in the art and/or are described herein.

The present disclosure also provides a method for assessing safety, genotoxicity and/or efficacy of a provirus construct or a modified provirus construct component, comprising: a) integrating the provirus construct into a stable cell line comprising a landing pad cassette of the disclosure integrated at a specific locus: b) selecting the cells comprising the integrated provirus construct, wherein selecting the cells comprising the integrated provirus construct comprises adding a compound to activate the suicide marker in the cells that do not have the integrated provirus construct; c) expanding the cells comprising the provirus construct to produce a stable cell line; and d) measuring the expression of the locus to determine the safety, genotoxicity and/or efficacy of the provirus construct or the modified provirus construct component.

The present disclosure also provides a method for assessing safety, genotoxicity and/or efficacy of a provirus construct or a modified provirus construct component, comprising: a) integrating the landing pad plasmid as described herein into the genome of a cell at a specific locus; b) selecting the cells comprising the landing pad plasmid, wherein selecting comprises selecting the cells expressing the detectable marker by fluorescence-activated cell sorting and/or selecting the cells resistant to antibiotic treatment by addition of an antibiotic; c) expanding the cells comprising the landing pad plasmid to produce a stable cell line; d) integrating the provirus construct into the stable cell line of step c), wherein the provirus construct is integrated between the first and the second site-specific recombination sites present in the stable cell line; e) selecting the cells comprising the integrated provirus construct, wherein selecting the cells comprising the integrated provirus construct comprises adding a compound to activate the suicide marker in the cells that do not have the integrated provirus construct; f) expanding the cells comprising the provirus construct to produce a stable cell line; and g) measuring the expression of the locus to determine the safety, genotoxicity and/or efficacy of the provirus or modified provirus construct component.

In one example, the modified provirus construct component is a regulatory element. In one example, the regulatory element is an insulator. For example, the insulator is in a forward orientation relative to a nucleotide sequence encoding a transgene of interest. In one example, the insulator is in a reverse orientation relative to a nucleotide e sequence encoding a transgene of interest. In another example, the regulatory element is a promoter. In yet another example, the regulatory element is an enhancer. In one example, the regulatory element is a lentiviral element.

For example, the method can be used to confirm provirus constructs with effective insulators. In one example, an insulator that reduces expression of the gene locus mRNA relative to cells without the insulator is an effective insulator. In one example, an insulator in a forward orientation relative to a nucleotide sequence encoding a transgene of interest that reduces expression of the gene locus mRNA relative to cells without the insulator is an effective insulator. In one example, an insulator in a reverse orientation relative to a nucleotide sequence encoding a transgene of interest that reduces expression of the gene locus mRNA relative to cells without the insulator is an effective insulator. In another example, an insulator does not reduce expression of gene locus mRNA relative to cells without the insulator is not an effective insulator. In another example, an insulator in a forward orientation relative to a nucleotide sequence encoding a transgene of interest does not reduce expression of gene locus mRNA relative to cells without the insulator is not an effective insulator. In another example, an insulator in a reverse orientation relative to a nucleotide sequence encoding a transgene of interest does not reduce expression of gene locus mRNA relative to cells without the insulator is not an effective insulator.

In one example, a promoter that reduces expression of the gene locus mRNA relative to cells without the promoter is not an effective promoter. In another example, a promoter does not reduce expression of gene locus mRNA relative to cells without the promoter is an effective promoter. In a further example, an enhancer that reduces expression of the gene locus mRNA relative to cells without the enhancer is not an effective enhancer. In another example, an enhancer does not reduce expression of gene locus mRNA relative to cells without the enhancer is an effective enhancer.

In one example, the method further comprises selecting an optimal provirus or modified provirus construct component.

As used herein, the term “optimal” refers to the most desirable or favourable outcome and not necessarily an outcome with the highest expression or blocking activity. For example, at least two proviruses or at least two modified provirus construct components can be compared to identify the optimal provirus or modified provirus construct component. In one example, the safety of the provirus or modified provirus construct component are compared to determine the optimal provirus or modified provirus construct component. For example, the optimal provirus or modified provirus construct component has improved safety relative to the other proviruses or modified provirus construct components. In one example, the genotoxicity of the provirus or modified provirus construct component are compared to determine the optimal provirus or modified provirus construct component. For example, the optimal provirus or modified provirus construct component has reduced genotoxicity relative to the other proviruses or modified provirus construct components. In one example, the efficacy of the provirus or modified provirus construct component are compared to determine the optimal provirus or modified provirus construct component. For example, the optimal provirus or modificd provirus construct component has enhanced efficacy relative to the other proviruses or modified provirus construct components. In one example, the method further comprises selecting a single clone comprising the integrated provirus construct from the expanded cells of the disclosure.

Selection of single clones allows for expansion of a selected clone comprising the integrated provirus construct.

In one example, the method further comprises expanding the single clone comprising the integrated provirus construct, wherein the single clone is isolated from the expanded cells produced by a method of the disclosure (i.e., the bulk cell assay described herein). It will be apparent to the skilled person that the present disclosure provides a bulk screening assay for expansion and selection of cells containing the provirus sequence, wherein following performing the bulk cell assay of the disclosure, single cell clonal selection is optionally performed. The skilled person will recognise that the present disclosure does not provide a method of performing single cell clonal selection without first performing the bulk cell assay of the disclosure. For example, in one example the method does not comprise single cell clonal selection. In another example, the method comprises performing the bulk cell assay of the disclosure without any further single cell clonal selection.

For example, a method for assessing safety, genotoxicity and/or efficacy of a provirus construct or a modified provirus construct component, comprises: a) integrating the landing pad plasmid as described herein into the genome of a cell at a specific locus: b) selecting the cells comprising the landing pad plasmid, wherein selecting comprises selecting the cells expressing the detectable marker by fluorescence-activated cell sorting and/or selecting the cells resistant to antibiotic treatment by addition of an antibiotic; c) expanding the cells comprising the landing pad plasmid to produce a stable cell line; d) integrating the provirus construct into the stable cell line of step c), wherein the provirus construct is integrated between the first and the second site-specific recombination sites present in the stable cell line; e) selecting the cells comprising the integrated provirus construct, wherein selecting the cells comprising the integrated provirus construct comprises adding a compound to activate the suicide marker in the cells that do not have the integrated provirus construct; f) selecting single cells comprising the provirus construct and expanding the single clones; and g) measuring the expression of the locus to determine the safety, genotoxicity and/or efficacy of the provirus construct or modified provirus construct component.

Detection of cells that do not have an integrated provirus construct or that have an incorrectly integrated provirus construct can be done by screening the cells for a detectable marker e.g., a fluorescent protein. In one example, the cells can be visualised using FACS and the cells that express the detectable marker can be visualised.

In one example, the landing pad cassette comprises a nucleotide sequence encoding an cGFP, the host cell comprises a nucleotide sequence encoding a mCherry and the provirus construct comprises a nucleotide sequence encoding an mScarlet. In one example, the cells comprising the landing pad cassette can be selected against using FACS to remove cells expressing eGFP. In one example, the cells comprising the provirus construct can be selected for using FACS to select cells expressing mScarlet. In one example, the cells comprising the provirus construct can be visualised using FACS to visual the cells expressing mScarlet. In one example, the cells not comprising the provirus construct can be visualised using FACS to visual the cells expressing eGFP.

### Use

In one example, the stable cell line described herein is used for producing an enveloped virus in a cell culture system e.g., for gene therapy.

The term “gene therapy” as used herein refers to a general method for treating a pathologic condition in a subject by inserting an exogenous nucleic acid into an appropriate cell(s) within the subject. The nucleic acid is inserted into the cell in such a way as to maintain its functionality, e.g., maintain the ability to express a particular polypeptide. In certain cases, insertion of the heterologous nucleic acid results in the expression of a therapeutically effective amount of a particular polypeptide.

As used herein, the term “subject” shall be taken to mean any animal including humans, for example a mammal. Exemplary subjects include but are not limited to humans and non-human primates. For example, the subject is a human.

The invention is further disclosed in the following numbered paragraphs:
1. A landing pad cassette comprising:
   a) a nucleotide sequence comprising a promoter operably linked to a nucleotide sequence encoding a detectable marker and/or a nucleotide sequence encoding a selection marker;
   b) a nucleotide sequence encoding a suicide marker; and
   c) a nucleotide sequence comprising a first site-specific recombination site and a nucleotide sequence comprising a second site-specific recombination site.
2. The landing pad cassette of paragraph 1, wherein the cassette comprises, in order from 5′ to 3′:
   a) a nucleotide sequence comprising a first site-specific recombination site;
   b) a nucleotide sequence comprising a promoter operably linked to a nucleotide sequence encoding a detectable marker and/or a nucleotide sequence encoding a selection marker;
   c) a nucleotide sequence encoding a suicide marker; and
   d) a nucleotide sequence comprising a second site-specific recombination site.
3. The landing pad cassette of paragraph 2, wherein:
   a) the first and/or second site-specific recombination sites are attP recombination sites; and/or
   b) the promoter is selected from the group consisting of a cytomegalovirus (CMV) promoter, a CMV enhancer, a murine leukemia virus-derived (MND) promoter, a simian virus 40 (SV40) promoter with enhancer, a polyubiquitin C gene (UBC) promoter, a phosphoglycerate kinase (PGK) promoter, a elongation factor-1 alpha (EF1A) promoter, a human β-actin (hACTB) promoter, a 7SK promoter, a cytomegalovirus immediate-early enhancer/chicken β-actin (CAG) promoter and combinations thereof; and/or
   c) the detectable marker is selected from the group consisting of an enhanced green fluorescent protein (eGFP), a red fluorescent protein (mCherry or mScarlet), a yellow fluorescent protein, a cyan fluorescent protein or combinations thereof; and/or
   d) the suicide marker is selected from the group consisting of Herpes Simplex Virus-1 thymidine kinase (HSV-TK), thymidine kinase (TK), caspase-9, caspase-8, purine nucleoside phosphorylase, uracil phosphoribosyl transferase, cytosine deaminase and combinations thereof; and/or
   e) the selection marker is selected from the group consisting of a neomycin resistance gene, a hygromycin resistance gene, a puromycin N-acetyl-transferase, a histidinol dehyrogenase, a zeocin resistance gene, a bleomycin resistance gene, a blasticidin S deaminase and combinations thereof.
4. The landing pad cassette of paragraph 3, wherein:
a) the first and the second site-specific recombination sites are attP recombination sites; and/or
b) the nucleotide sequence comprising the CMV promoter comprises a sequence set forth in SEQ ID NO: 3 or 4, or the nucleotide sequence comprising the MND promoter comprises a sequence set forth in SEQ ID NO: 5; and/or
c) the nucleotide sequence encoding the eGFP comprises a sequence set forth in SEQ ID NO: 7; and/or
d) the nucleotide sequence encoding the HSV-TK comprises a sequence set forth in SEQ ID NO: 6; and/or
e) the nucleotide sequence encoding the puromycin N-acetyl-transferase comprises a sequence set forth in SEQ ID NO: 8.

5. The landing pad cassette of any one of paragraphs 2 to 4, further comprising:
a) a nucleotide sequence comprising a linker located between the nucleotide sequence encoding the detectable marker and the nucleotide sequence encoding the suicide marker, wherein the linker is an internal ribosome entry site (IRES) or encodes a 2A self-cleaving peptide, and wherein the 2A self-cleaving peptide is selected from the group consisting of a P2A, T2A, E2A and a F2A; and/or
b) a nucleotide sequence comprising a polyA signal located 3′ of the nucleotide sequence encoding the suicide marker, wherein the polyA signal is selected from the group consisting of a SV40 polyA, SVLP polyA, hGH polyA, BGH polyA and rbGlob polyA; and/or
c) a nucleotide comprising a woodchuck hepatitis virus post-transcriptional regulatory element (WPRE) located 3′ of the nucleotide sequence encoding the suicide marker.

6. The landing pad cassette of paragraph 5, wherein:
a) the nucleotide sequence encoding the P2A comprises a sequence set forth in any one of SEQ ID NOs: 10 to 13, or the nucleotide sequence encoding the T2A comprises a sequence set forth in SEQ ID NO: 15 or 16; and/or
b) the nucleotide sequence comprising the SV40 polyA signal comprises a sequence set forth in SEQ ID NO: 17;
c) the nucleotide sequence comprising the WPRE comprises a sequence set forth in SEQ ID NO: 20; and/or
d) the nucleotide sequence comprising the BGH polyA signal comprises a sequence set forth in SEQ ID NO: 55.

7. A landing pad cassette comprising, in order from 5′ to 3′:
a) a nucleotide sequence comprising an attP (GT) recombination site;
b) a nucleotide sequence comprising a CMV promoter or a nucleotide sequence comprising a MND promoter;
c) a nucleotide sequence encoding an enhanced green fluorescent protein (eGFP) detectable marker;
d) a nucleotide sequence encoding a P2A self-cleaving peptide linker;
e) a nucleotide sequence encoding a Herpes Simplex Virus-1 thymidine kinase (HSV-TK) suicide marker;
f) a nucleotide sequence comprising a WPRE;
g) a nucleotide sequence comprising a BGH polyA signal; and
h) a nucleotide sequence comprising an attP (GA) site.
8. A landing pad plasmid comprising the landing pad cassette of any one of paragraphs 1 to 7, comprising a nucleotide sequence comprising a 5′ homology arm (HA) and a nucleotide sequence comprising a 3′ HA.
9. The landing pad plasmid of paragraph 8, further comprising one or more of:
   a) a nucleotide sequence comprising one or more additional promoters, wherein the one or more additional promoters is selected from the group consisting of a CMV promoter, a CMV enhancer, a MND promoter, a SV40 promoter with enhancer, a UBC promoter, a PGK promoter, a EF1A promoter, a hACTB promoter, a CAGG promoter and combinations thereof; and/or
   b) a nucleotide sequence encoding one or more additional detectable markers, wherein the one or more additional detectable markers is selected from the group consisting of an enhanced green fluorescent protein (eGFP), a red fluorescent protein (mCherry or mScarlet), a yellow fluorescent protein, a cyan fluorescent protein and combinations thereof; and/or
   c) a nucleotide sequence comprising an additional polyA signal, wherein the additional polyA signal is selected from the group consisting of a SV40 poly A, SVLP poly A, hGH polyA, BGH polyA and rbGlob polyA; and/or
   d) a nucleotide sequence comprising a viral origin of replication sequence; and/or
   e) a nucleotide sequence encoding an antibiotic resistance gene operably linked to a nucleotide sequence comprising a promoter.
10. The landing pad plasmid of paragraph 9, wherein:
   a) the nucleotide sequence comprising the 5′ HA comprises a sequence set forth in SEQ ID NO: 24 or 26; and/or
   b) the nucleotide sequence comprising the 3′ HA comprises a sequence set forth in SEQ ID NO: 25 or 27; and/or
   c) the nucleotide sequence comprising the SV40 promoter with enhancer comprises a sequence set forth in SEQ ID NO: 17.
   d) the nucleotide sequence comprising the BGH polyA signal comprises a sequence set forth in SEQ ID NO: 55.
11. The landing pad plasmid of any one of paragraphs 8 to 10, wherein the landing pad plasmid comprises a sequence set forth in SEQ ID NO: 1 or SEQ ID NO: 2.
12. A landing pad plasmid comprising, in order from 5′ to 3′:
   a) a nucleotide sequence comprising a 5′ homology arm (HA);
   b) a nucleotide sequence comprising an attP (GT) recombination site;
   c) a nucleotide sequence comprising a CMV promoter or a nucleotide sequence comprising a MND promoter;
   d) a nucleotide sequence encoding an enhanced green fluorescent protein (eGFP) detectable marker;
   e) a nucleotide sequence encoding a P2A self-cleaving peptide linker;
   f) a nucleotide sequence encoding an Herpes Simplex Virus-1 thymidine kinase (HSV-TK) suicide marker;
   g) a nucleotide sequence comprising a WPRE;
   h) a nucleotide sequence comprising a BGH polyA signal;
   i) a nucleotide sequence comprising an attP (GA) recombination site;
   j) a nucleotide sequence comprising a 3′ HA;
   k) optionally a nucleotide sequence encoding a mCherry detectable marker;
   l) a nucleotide sequence comprising a pUC viral origin of replication sequence; and
   m) a nucleotide sequence encoding an Amp (R) gene operably linked to a nucleotide sequence comprising a bla promoter.
13. A method of stably integrating the landing pad cassette of any one of paragraphs 1 to 7 into the genome of a cell at a specific locus, wherein the landing pad is integrated at the specific locus using site-directed modification, and optionally further comprising selecting the cells comprising the landing pad plasmid.
14. The method of paragraph 13, wherein the specific locus is a leukemia oncogene (LMO2) locus, a MDS1 And EVIL Complex (MECOM) locus, a cyclin D2 (CCND2) locus, a B lymphoma Mo-MLV insertion region 1 homolog (BMI1) locus or a meningioma (disrupted in balanced translocation) 1 (MN1) locus, and optionally wherein the LMO2 locus is 33 kb upstream of the transcription start site (TSS) or 2 kb downstream of the TSS.
15. The method of paragraph 13 or 14, wherein selecting the cells comprising the landing pad cassette comprises selecting the cells resistant to antibiotic treatment by addition of an antibiotic, and optionally wherein the cells comprising the landing pad cassette are expanded to produce a stable cell line.
16. A stable cell line comprising the landing pad cassette of any one of paragraphs 1 to 7, preferably wherein the cell line is a Jurkat cell line or a K562 cell line.
17. A provirus construct comprising:
   a) a nucleotide sequence comprising one of more site-specific recombination sites;
   b) a nucleotide sequence comprising a 5′ long terminal repeat (LTR);
   c) a nucleotide sequence encoding a transgene of interest operably linked to a promoter; and
   d) a nucleotide sequence comprising a 3′ LTR comprising an insulator.
18. The provirus construct of paragraph 17, further comprising one or more of:
   a) a nucleotide sequence encoding a suicide marker operably linked to a nucleotide sequence comprising a promoter; and/or
   b) a nucleotide sequence comprising a polyA signal.
19. The provirus construct of paragraph 17 or 18 comprising, in order from 5′ to 3′:
   a) a nucleotide sequence comprising a first site-specific recombination site, wherein the first site-specific recombination site is a attB (GT) recombination site:
   b) a nucleotide sequence comprising a 5′ LTR;
   c) a nucleotide sequence encoding a transgene of interest operably linked to a promoter, wherein the promoter is selected from the group consisting of a CMV promoter, a CMV enhancer, a MND promoter, a SV40 promoter with enhancer, a UBC promoter, a PGK promoter, a EF1A promoter, a hACTB promoter and a CAG promoter;
   d) a nucleotide comprising a woodchuck hepatitis virus post-transcriptional regulatory element (WPRE);
   e) a nucleotide sequence comprising a 3′ LTR comprising an insulator, wherein the insulator is a chicken hypersensitive site-4 (cHS4) insulator and optionally wherein the nucleotide sequence comprising the cHS4 insulator comprises a sequence set forth in any one of SEQ ID NOs: 29 to 31; and
   f) a nucleotide sequence comprising a second site-specific recombination site, wherein the second site-specific recombination site is a attB (GA) recombination site.
20. A provirus construct comprising, in order from 5′ to 3′:
   a) a nucleotide sequence comprising a attB (GT) recombination site;
   b) a nucleotide sequence comprising a 5′ long terminal repeat (LTR);
   c) a nucleotide sequence comprising lentiviral elements;
   d) a nucleotide sequence comprising a MND promoter operably linked to a nucleotide sequence comprising a transgene of interest;
   e) a nucleotide sequence comprising a woodchuck hepatitis virus post-transcriptional regulatory element (WPRE);
   f) a nucleotide sequence comprising a 3′ LTR comprising a cHS4 insulator; and
   g) a nucleotide sequence comprising a attB (GA) recombination site.
21. A provirus vector comprising the provirus construct of any one of paragraphs 17 to 20.
22. The provirus vector of paragraph 21, wherein the vector further comprises:
   a) a nucleotide sequence encoding a suicide marker operably linked to a nucleotide sequence comprising a promoter, wherein the suicide marker is selected from the group consisting of a Herpes Simplex Virus-1 thymidine kinase (HSV-TK), a thymidine kinase (TK), a caspase-9, a caspase-8, a purine nucleoside phosphorylase, an uracil phosphoribosyl transferase or a cytosine deaminase and wherein the promoter is a CMV promoter, a CMV enhancer, a MND promoter, a SV40 promoter with enhancer, an UBC promoter, a PGK promoter, an EF1A promoter, a hACTB promoter and a CAG promoter; and
   b) a nucleotide sequence comprising a polyA signal, wherein the polyA signal is selected from the group consisting of a SV40 polyA, SVLP polyA, hGH polyA. BGH polyA or rbGlob polyA.
23. The provirus vector of paragraph 22, wherein the vector comprises a nucleotide sequence comprising a CMV promoter operably linked to a nucleotide sequence encoding a Herpes Simplex Virus-1 thymidine kinase (HSV-TK) suicide marker; and a nucleotide sequence comprising a SV40 polyA signal.
24. The provirus vector of any one of paragraphs 21 to 23, wherein the provirus vector comprises a sequence set forth in SEQ ID NO: 48.
25. A method of stably integrating the provirus construct of any one of paragraphs 17 to 20 into the stable cell line of paragraph 16, wherein the provirus construct is integrated between the first and the second site-specific recombination sites present in the stable cell line.
26. The method of paragraph 25, wherein the method comprises transfecting the provirus construct into the cell line in a cell culture in the presence of a recombinase, wherein the recombinase and provirus construct are added to the cell culture at a ratio of at least 3 to 1, and optionally wherein the recombinase is a serine recombinase Bxb1.
27. The method of paragraphs 25 or 26, wherein the method further comprises selecting the cells comprising the integrated provirus construct by addition of a compound that activates the suicide marker in the cells that do not have the integrated provirus construct, optionally wherein the compound is ganciclovir (GCV).
28. A stable cell line comprising the provirus construct of any one of paragraphs 17 to 20.
29. Use of the stable cell line of paragraph 28 for production of an enveloped virus.
30. A method of developing an improved cell line for use as a screening tool for components of a modified provirus construct component.
31. A method of optimising a provirus construct, comprising integrating a provirus construct into a cell line comprising the landing pad cassette of any of one of paragraphs 1 to 7, and assessing the activity of the integrated construct.
32. A method for assessing safety, genotoxicity and/or efficacy of a provirus construct, comprising:
   a) integrating the landing pad cassette of any one of paragraphs 1 to 7 into the genome of a cell at a specific locus;
   b) selecting the cells comprising the landing pad cassette, wherein selecting comprises selecting the cells resistant to antibiotic treatment by addition of an antibiotic;
   c) expanding the cells comprising the landing pad cassette to produce a stable cell line;
   d) integrating the provirus construct into the stable cell line of step c), wherein the provirus construct is integrated between the first and the second site-specific recombination sites present in the stable cell line;
   e) selecting the cells comprising the integrated provirus construct, wherein selecting the cells comprising the integrated provirus construct comprises adding a compound to activate the suicide marker in the cells that do not have the integrated provirus construct;
   f) expanding the cells comprising the provirus construct to produce a final cell line; and
   g) measuring the expression of the locus to determine the safety, genotoxicity and/or efficacy of the provirus or components of the provirus.
33. The method of paragraph 32, wherein the method further comprises comparing the safety, genotoxicity and/or efficacy of at least two proviruses or modified provirus construct components to identify an optimal optimal provirus or modified provirus construct component.
34. The method of paragraph 32 or 33, wherein the component of the provirus is an insulator, a promoter, an enhancer, a lentiviral element or combinations thereof, preferably wherein the component is an insulator.
35. The method of any one of paragraphs 32 to 34, wherein the provirus construct is a construct of any one of paragraphs 17 to 20.
36. The method of any one of paragraphs 32 to 35, wherein the specific locus is a leukemia oncogene (LMO2) locus, a MDS1 And EVI1 Complex (MECOM) locus, a cyclin D2 (CCND2) locus, a B lymphoma Mo-MLV insertion region 1 homolog (BMI1) locus or a meningioma (disrupted in balanced translocation) 1 (MN1) locus, and optionally wherein the LMO2 locus is 33 kb upstream of the transcription start site (TSS) or 2 kb downstream of the TSS.

The present disclosure includes the following non-limiting Examples.

### Examples

### Example 1-Landing Pad Construction and Insertion

Cell lines comprising a landing pad were prepared to enable targeted integration of provirus constructs. Specifically, two sites known to have caused leukemic situations in patients were targeted.

A plasmid was used to knock-in a landing pad cassette into the targeted sites using CRISPR-Cas9 and homology directed repair (HDR). A first-generation landing pad plasmid was constructed using pcDNA3.1 (+). Required landing pad components were inserted between the Mlul and the BstBI site. The SV40 polyA site, pUC origin of replication and the AmpR gene were retained. Within the insert, CMV and SV40 promoters as well as the BGH polyA signal were re-introduced. The f1 origin was not re-introduced and was thus removed from the construct. A second-generation landing pad plasmid was also constructed using the pcDNA3.1 (+). Required landing pad components were inserted between the Mlul and the PciI site. Unlike in the first-generation constructs, the SV40 polyA site was also removed from the plasmid backbone. Within the second generation insert, an MND promoter and a BGH polyA signal were introduced.

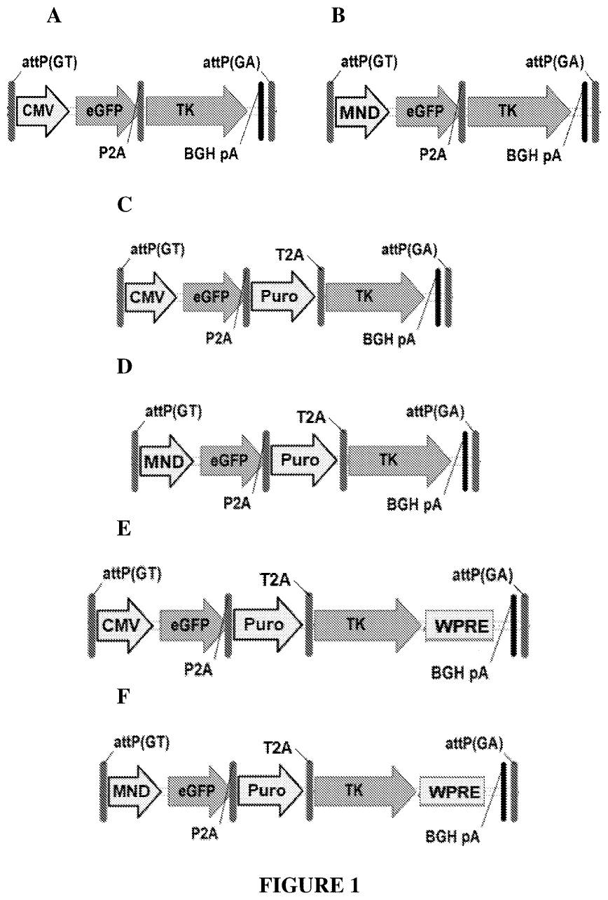

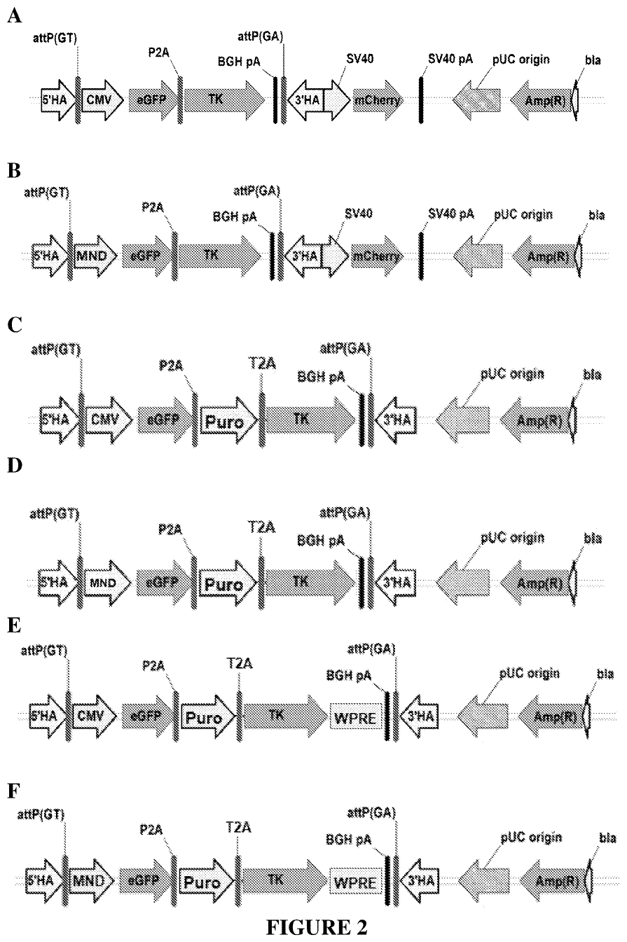

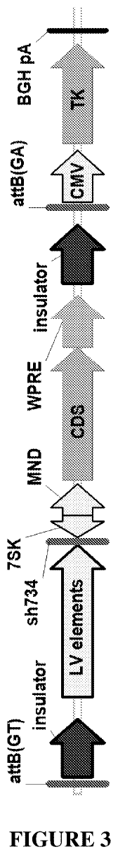

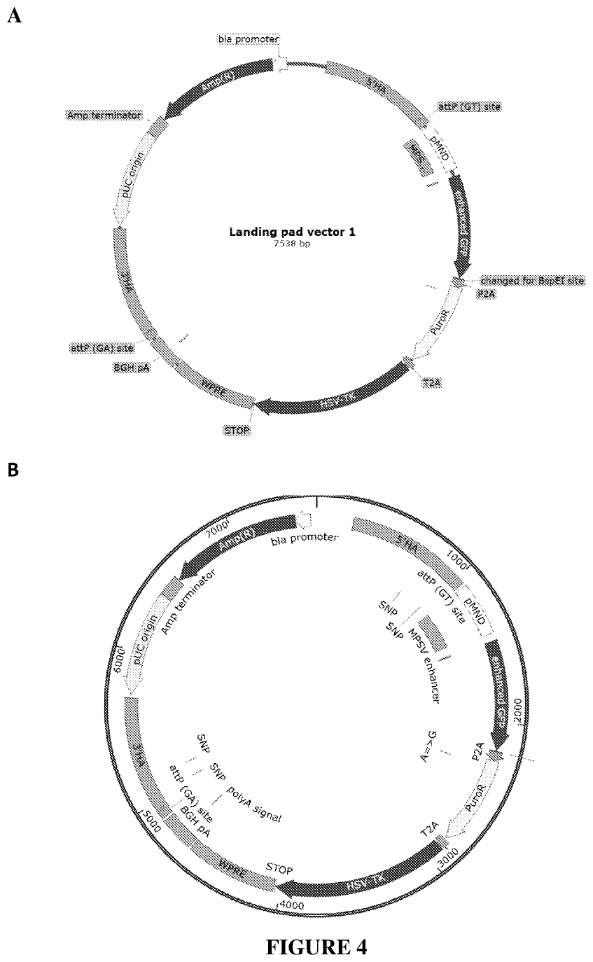

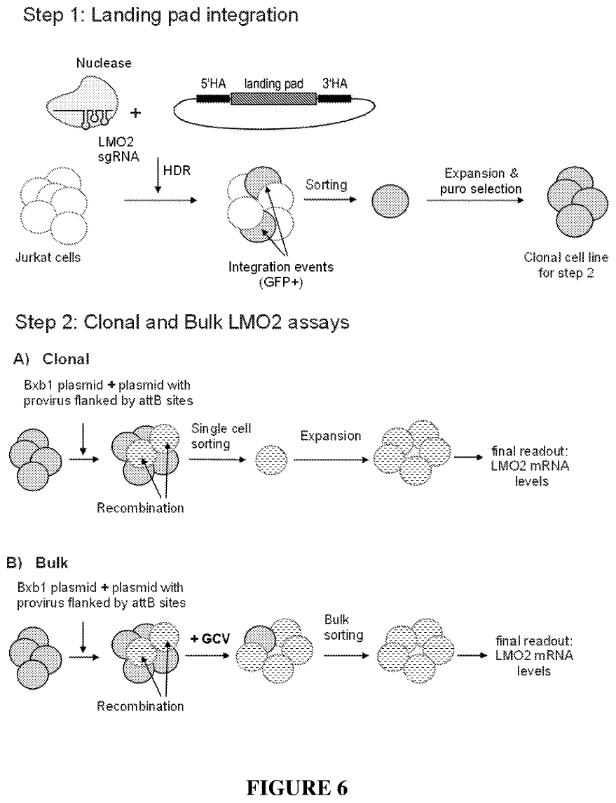

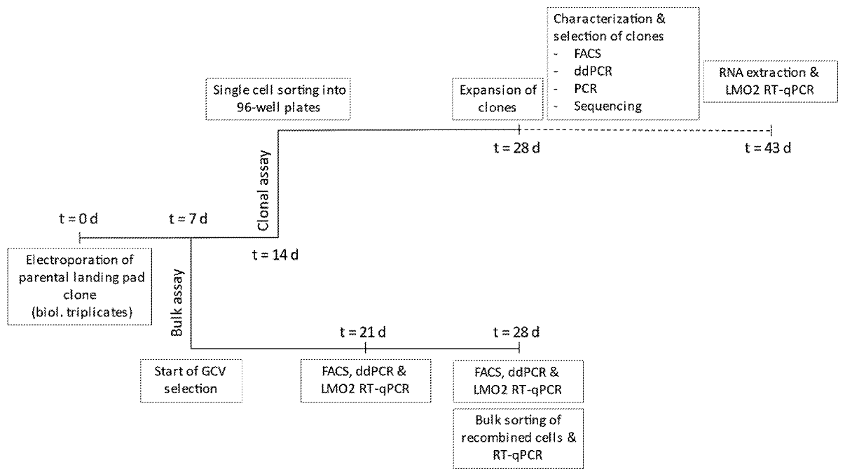

Two integration sites within the LMO2 gene were targeted:
Site 1: Patient WAS6, described in Braun et aL, 2014 (WAS clinical trial) had an integration site at about 33 kb upstream of the LMO2 transcription start site.
Site 2: Patient P4, described in Hacein-Bey-Albina et aL, 2003 (SCID-X1 clinical trial) had an integration site at about 2 kb downstream of the LMO2 transcription start site, within intron 1.
   gRNAs and homology arm sequences were designed for use in Jurkat T cells. Regions around the insertion sites were sequenced accordingly, gRNAs were selected based on distance to the insertion site and/or based on calculated on-target and off-target scores. Two gRNAs were chosen targeting Site 1 (LMO2_g1: SEQ ID NO: 22, and LMO2_g2: SEQ ID NO: 23), one gRNA targeting integration Site 2, as described in Goodman et aL, 2018, was chosen (LMO2_gRNA5: SEQ ID NO: 21) (FIG. 6A).
The landing pad cassette consisted of the following expressed transgenes (FIG. 1):
   1) GFP reporter;
   2) Suicide gene (e.g., Herpes Simplex Virus thymidine kinase); and
   3) Puromycin resistance gene.
The complete plasmid consisted of the following components (FIGS. 2 to 4):
   1) Cloning site for 5′ homology arm (for HDR);
   2) att site;
   3) Cassette (Promoter, GFP gene, suicide gene, resistance gene);
   4) WPRE element
   5) BGH polyA signal
   6) att site;
   7) Cloning site for 3′ homology arm (for HDR); and
   8) optionally an mCherry reporter for plasmid marking.

The plasmid design allows for amplification in bacteria (ori. Amp/Kan resistance etc.) and for expression of GFP, optionally mCherry, Puromycin resistance and suicide genes in human cell lines (e.g., MND promoter with BGH poly A or CMV promoter and SV40 polyA signal).

The GFP reporter enables cells with the integrated cassette (or with the transfected plasmid) to be visualized. The optional mCherry reporter allows for distinction between cells with a non-integrated plasmid and cells with the integrated cassette. The optional antibiotic resistance gene allows for selection for cells with an integrated and functional cassette leading to expression of the proteins. Once the cassette was integrated, the LoxP or att sites allow for replacement of the cassette by another component of interest. Inclusion of a suicide gene in the landing pad cassette, i.e., TK, allows for bulk selection of cells in which the cassette has been replaced (i.e., by a provirus or other component of interest) and avoids the need for single cell clone selection. These cells should also become GFP negative.

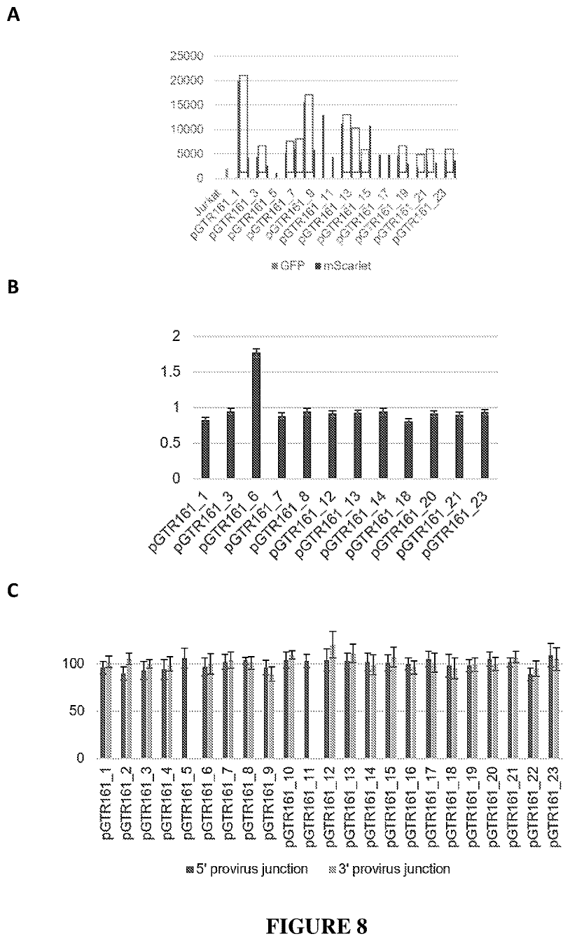

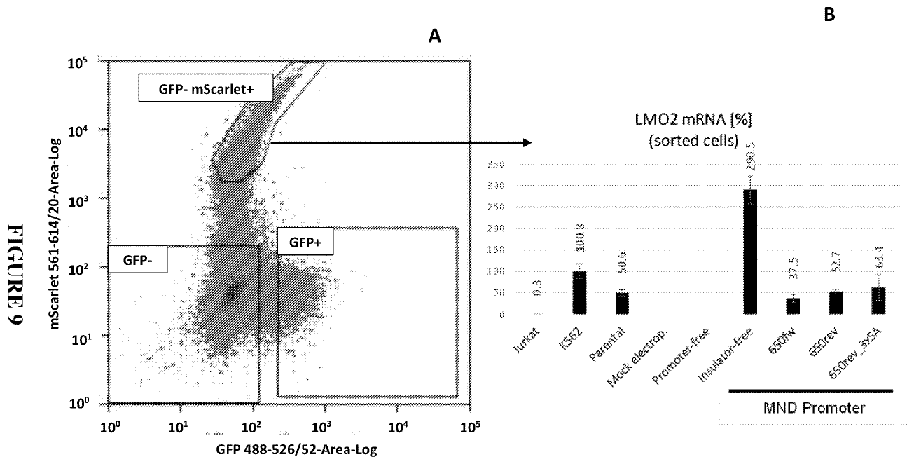

Clones were assessed for EGFP expression using FACS to identify clones with uniform persistent expression. Vector copy number was assessed by Droplet Digital PCR (ddPCR) and clones with a single integrated landing pad were selected (FIG. 8).

PCR and Sanger sequencing was used to confirm correct junctions to genome and the sequencing of LMO2 locus.

### Example 2-Provirus Construction

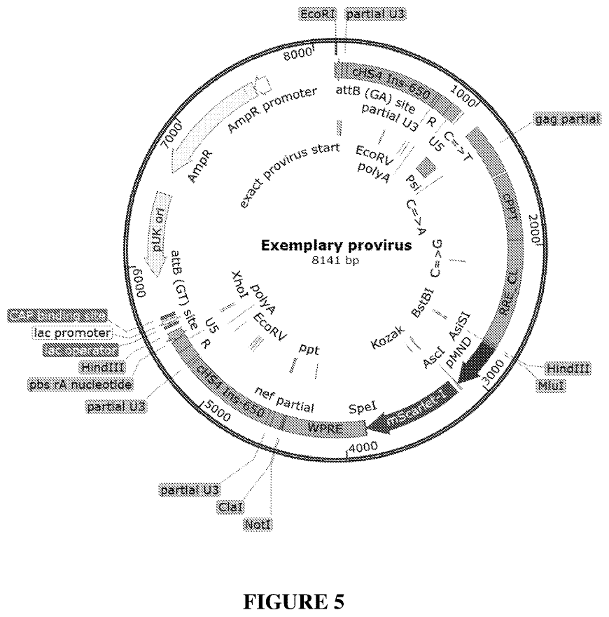

The pUC57 backbone was used for exemplary provirus constructs because of the restriction site landscape of the backbone. The following components were cloned into this backbone (between pUC57 EcoRI and SphI sites) (FIG. 5):
i. attB (GT/GA) site;
ii. 5′ LTR containing an insulator of interest;
iii. BstBI and NotI restriction sites for cloning in a gene of interest (i.e., therapeutic gene of interest, a reporter gene or other);
iv. 3′ LTR containing an insulator;
v. attB (GA/GT) site; and
vi. optionally CMV promoter and HSV-TK ORF with BGH polyA signal.

### Example 3-Validation Assays

### Cell Prep

Cells comprising a landing pad were thawed in a water bath at 37° C. before being resuspended in RPMI medium without Puromycin, and incubated under cell cultivation conditions. After 24 hours cell density and viability was recorded prior to addition of Puromycin (2 μg/ml) and further incubation under cell cultivation conditions.

After further cultivation for 24-48 hours, cells were centrifuged and supernatant removed before being resuspended in complete media with Puromycin (2 μg/ml final concentration). Complete media or medium refers to RPMI (25 mM HEPES and GlutaMax)+10% FBS+1% Pen/Strep. Cells were maintained in complete media with puromycin (2 μg/ml final cone) under cell cultivation conditions until used for electroporation.

Electroporation with Provirus Constructs

Thawed plasmid stocks were vortexed prior to being diluted and combined to a plasmid-mix with a target ratio 3:1 (concentration of Plasmid Provirus plasmid 750 ng/μL: B×b1 plasmid 250 ng/μL).

Cell samples were centrifuged 200 g for 5 minutes at room temperature prior to combining with the plasmid-mix as per Table 2:

TABLE 2
Plasmid Mix

| | Final plasmid | Volume [μl] |
|---|---|---|
| | amount | (for 1 sample) |
| Plasmid mix (use water for Mock) | 1 μg | 2 |
| Cell suspension | 0.25*10\hat{ }6 cells | 10 |

Combined cell suspension plasmid-mix was electroporated at 1325 V. 10 ms pulse width, 3 pulses. After electroporation, cells were incubated at 37° C., 5% CO₂. Density and viability were measured at regular intervals and fresh RPMI medium (w/o Puromycin) added as required.

Bulk cell Assay-GCV Bulk Selection Protocol

7 days post-transfection, cells with integrated provirus were treated with GCV to remove clones with intact landing pad (i.e., clones with no provirus or integration into a locus other than the landing pad). Specifically, viable cells were centrifuged, supernatant was removed and cells subsequently resuspended in complete medium containing 1 μg/ml GCV. GCV-treated cells were kept in culture and fresh medium containing 1 μg/mL GCV added as required.

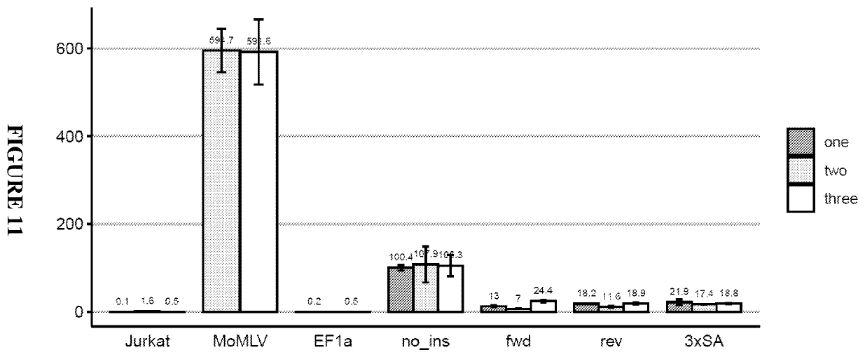

After around 13-15 days under GCV selection (i.e. 20-22 days post-transfection), FACS analysis was conducted to assess recombination efficiency if a suitable reporter transgene was included in the provirus. Optionally, mScarlet-I positive cells (if mScarlet-I reporter was included in the provirus transgene) were sorted in bulk from the population to reduce background from non-recombined cells. Genomic DNA was extracted and ddPCR analysis with assays spanning the genome-provirus junctions was conducted to measure recombination efficiency at the DNA level. RNA was extracted and LMO2 mRNA gene expression was analysed by quantitative real-time reverse transcriptase-polymerase chain reaction (qRT-PCR) after 14 days under GCV selection for bulk cells (FIGS. 6B, 7 and 10).

### Single Clone Assay Clone Selection Protocol

14 days post-transfection, single cells were sorted into 96-well plates. In a first selection, approximately 20-50 clones/constructs were expanded for a further 14 days under cell cultivation conditions. 28-days post transfection, clones were subsequently characterized by FACS to confirm mScarlet expression (if included in the provirus transgene) and the absence of EGFP (from the landing pad) expression and ddPCR to confirm provirus junctions.

In a second selection, approximately 10-15 clones/constructs were selected from the initial analysis. Selected clones were further characterized by ddPCR to confirm vector copy number (VCN) and the sequence of the junctions. In a final selection, clones with correct junctions and having a VCN of 1 were selected. RNA was extracted and LMO2 mRNA gene expression was analysed for final selected clones using qRT-PCR (FIGS. 6B and 7). In some examples. Multiplex 1-step RT PCR was specifically used.

### Results

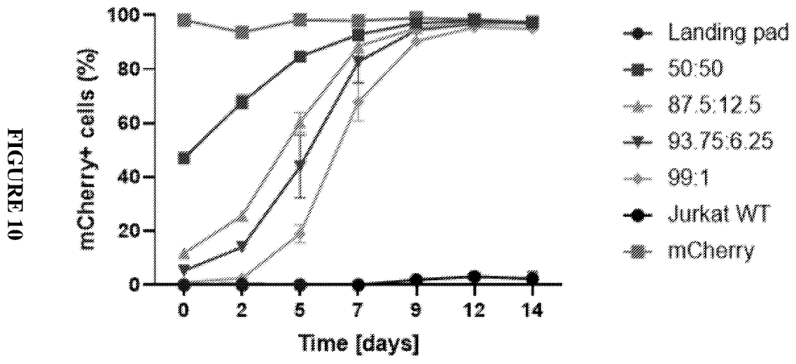

In brief, Jurkat cell lines having a targeted integration of a provirus within the promoter or the first intron of the LMO2 were used to assess vector constructs (FIG. 9 and Table 3) (Ryu et aL, 2008; Zhou et aL, 2010). A MoMLV provirus was used as a positive control for LMO2 activation. A provirus construct without promoter or with an EF1 a promoter were used as negative controls without LMO2 activation. Lentiviral vector provirus constructs with an MND promoter driving an mScarlet-I reporter transgene and harboring different insulator sequences in the LTRs were tested in this assay. A lentiviral provirus construct with an MND promoter driving an mScarlet-I reporter transgene but lacking an insulator (“insulator-free”) was used as a reference (FIG. 11 and Table 4). Where an insulator sequence exhibits effective enhancer-blocking function, a LMO2 expression level lower than LMO2 expression in the insulator-free provirus is observed. Where an insulator sequence exhibits reduced or no enhancer-blocking function, LMO2 expression levels comparable to an uninsulated construct, or at least higher than an alternative effective insulator sequence, may be observed. Such analysis enables a direct functional comparison of alternative vector components, for example, alternative insulator sequences. It is envisaged that such an analysis could similarly be applied to other vector components, such as promoters.

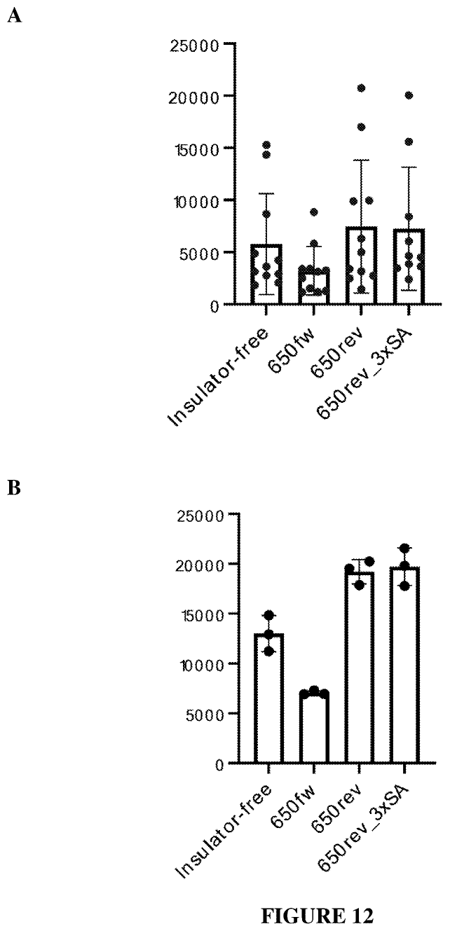

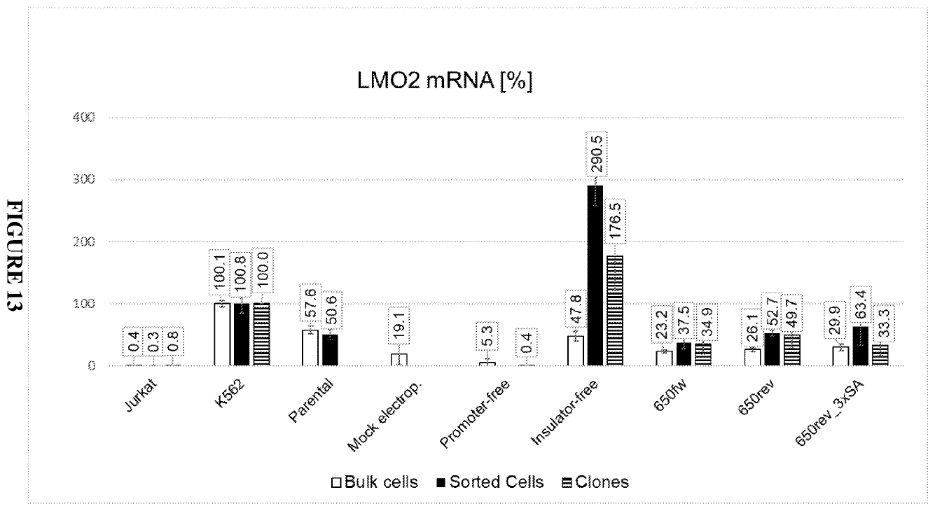

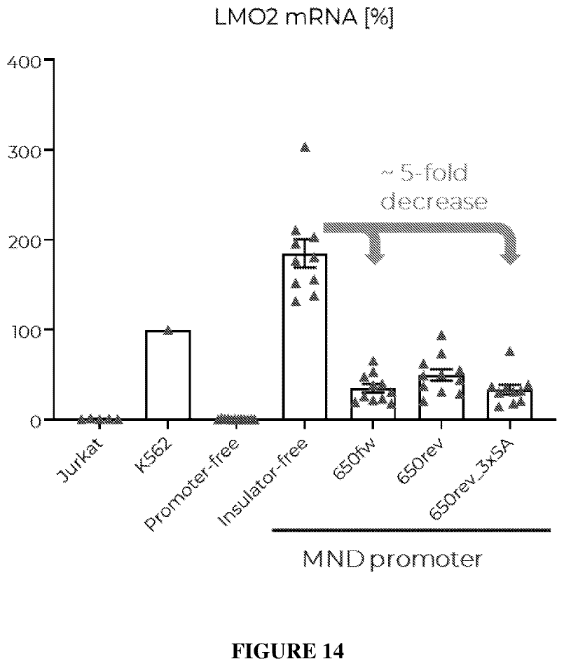

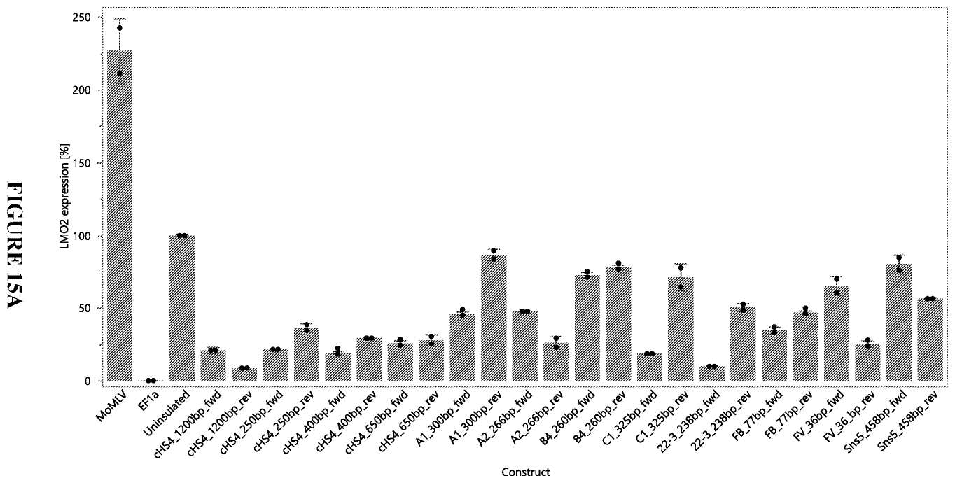

Analysis of LMO2 expression from both a single clone assay and bulk cell assay were completed. The bulk cell assay, utilizing a landing pad construct comprising a suicide gene (i.e. TK), enable efficiency selection of desired clones. Results were comparable to the single cell clone assay. However, the bulk cell assay had significant advantages. Specifically, the bulk cell assay may be completed in reduced time (approximately 21 days), requires less work as no clones need to be characterized, and/or benefits from higher accuracy and lower variability due to collective measure of thousands of clones (FIGS. 12 to 14). These advantages enable the bulk cell assay to be utilised as a higher throughput screening tool to inform construct design.

TABLE 3
LMO2 mRNA expression in Jurkat cells transfected
with plasmids of the disclosure

| Sample | LMO2 mRNA [%] (sorted cells) |
|---|---|
| Jurkat | 0.3 |
| K562 | 100.8 |
| Parental | 50.6 |
| Mock electroporation | — |
| Promoter-free | — |
| Insulator-free | 290.5 |
| 650fw | 37.5 |
| 650rev | 52.7 |
| 650rev_3xSA | 63.4 |

TABLE 4
LMO2 mRNA levels in sorted mScarlet+ cells
with positive control: MoMLV gammaretrovirus;
and negative control: EF1a short promoter

| Bulk cell assay | LMO2 mRNA levels in sorted mScarlet+ cells | | |
|---|---|---|---|
| Sample | Rep 1 | Rep 2 | Rep 3 |
| Jurkat | 0.1 | 1.6 | 0.5 |
| MoMLV | — | 594.7 | 591.6 |
| EF1a | — | 0.2 | 0.5 |
| Insulator-free (no_ins) | 100.4 | 107.9 | 105.3 |
| 650fw (fwd) | 13 | 7 | 24.4 |
| 650rev (rev) | 18.2 | 11.6 | 18.9 |
| 650rev_3xSA (3xSA) | 21.9 | 17.4 | 18.8 |

### Example 4-Insulator Screening

The methods described in Example 3 were used to screen a variety of known insulators, in both a forward and reverse orientation relative to a nucleotide sequence encoding a transgene of interest, to compare insulator effectiveness and demonstrate the efficacy of the bulk cell assay.

A MoMLV provirus was used as a positive control for LMO2 activation. An uninsulated provirus construct and a provirus construct with an EF1a promoter were used as negative controls without LMO2 activation. Lentiviral vector provirus constructs with different insulator sequences, in forward and reverse positions relative to the gene of interest, were tested using the bulk cell assay.

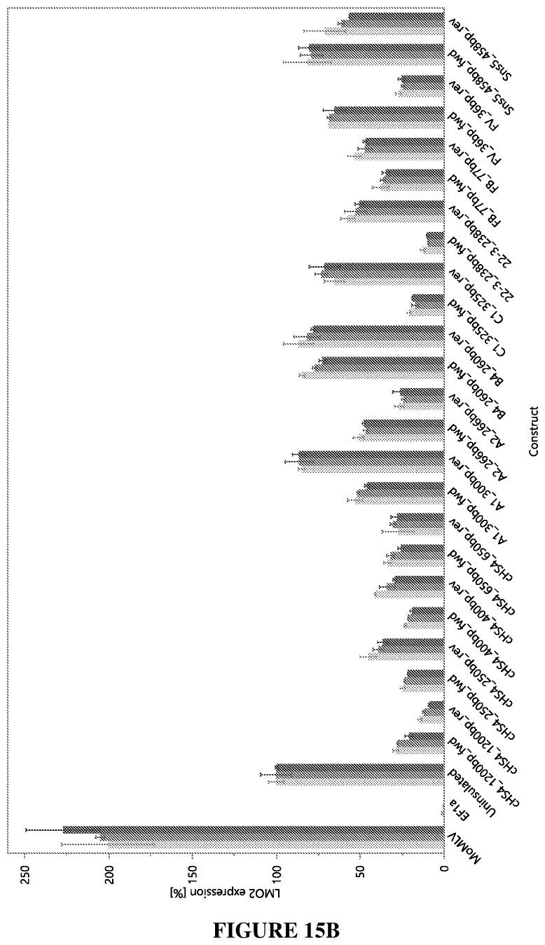

### Results

Where an insulator exhibits effective enhancer-blocking function, a LMO2 expression level lower than LMO2 expression in the insulator-free provirus (uninsulated construct) is observed. Where an insulator exhibits reduced or no enhancer-blocking function, LMO2 expression levels comparable to an uninsulated construct, or at least higher than an alternative effective insulator sequence, may be observed. Such analysis enables a direct functional comparison of alternative insulator sequences (FIG. 15 and Tables 5 and 6).

It is also envisaged that such an analysis could similarly be applied to optimise vector design and guide selection of combinations of vector components, such as promoters, insulators, enhancers and/or transgenes.

TABLE 5
Percentage LMO2 expression for range of insulators relative to
uninsulated control construct (tabulated data from FIG. 15A)

| | | | % relative LMO2 |
|---|---|---|---|
| Insulator | Reference | Orientation | expression* |
| MoMLV | MoMLV | N/A | 202.76 |
| positive | | | |
| control | | | |
| Ef1a | EF1a | N/A | 0.60 |
| negative | | | |
| control | | | |
| Uninsulated | Uninsulated | N/A | 85.37 |
| construct^{#} | | | |
| cHS4 1200 | cHS4_1200 bp_fwd | Forward | 25.94 |
| bp | | | |
| cHS4 1200 | cHS4_1200 bp_rev | Reverse | 11.82 |
| bp | | | |
| cHS4 250 | cHS4_250 bp_fwd | Forward | 23.38 |
| bp | | | |
| cHS4 250 | cHS4_250 bp_rev | Reverse | 40.45 |
| bp | | | |
| cHS4 400 | cHS4_400 bp_fwd | Forward | 21.38 |
| bp (SEQ ID | | | |
| NO: 69) | | | |
| cHS4 400 | cHS4_400 bp_rev | Reverse | 34.99 |
| bp (SEQ ID | | | |
| NO: 70) | | | |
| cHS4 650 | cHS4_650 bp_fwd | Forward | 30.58 |
| 3SA bp | | | |
| (SEQ ID | | | |
| NO: 28) | | | |
| cHS4 650 | cHS4_650 bp_rev | Reverse | 28.75 |
| 3SA bp | | | |
| (SEQ ID | | | |
| NO: 56) | | | |
| A1 300 bp | A1_300 bp_fwd | Forward | 50.23 |
| A1 300 bp | A1_300 bp_rev | Reverse | 86.11 |
| A2 266 bp | A2_266 bp_fwd | Forward | 48.59 |
| A2 266 bp | A2_266 bp_rev | Reverse | 25.85 |
| C1 325 bp | C1_325 bp_fwd | Forward | 19.07 |
| C1 325 bp | C1_325 bp_rev | Reverse | 70.06 |
| B4 260 bp | B4_260 bp_fwd | Forward | 78.29 |
| B4 260 bp | B4_260 bp_rev | Reverse | 82.26 |
| 22-3 238 bp | 22-3_238 bp_fwd | Forward | 10.80 |
| 22-3 238 bp | 22-3_238 bp_rev | Reverse | 53.68 |
| FB 77 bp | FB_77 bp_fwd | Forward | 36.45 |
| FB 77 bp | FB_77 bp_rev | Reverse | 49.30 |
| Foamy | FV_36 bp_fwd | Forward | 57.43 |
| Virus (FV) | | | |
| 36 bp | | | |
| Foamy | FV_36 bp_rev | Reverse | 25.82 |
| Virus (FV) | | | |
| 36 bp | | | |
| Sns5 458 | Sns5_458 bp_fwd | Forward | 72.45 |
| bp | | | |
| Sns5 458 | Sns5_458 bp_rev | Reverse | 77.59 |
| bp | | | |

*relative to uninsulated construct^{#}

TABLE 6
Percentage LMO2 expression for range of insulators relative to uninsulated
control construct (triplicate tabulated data from FIG. 15B)

| | | | % relative LMO2 expression* | | |
|---|---|---|---|---|---|
| | | | Replicate 1 | Replicate 2 | Replicate 3 |
| Insulator | Orientation | Reference | (D) | (E) | (F) |
| MoMLV positive | N/A | MoMLV | 200.40 | 205.12 | 80.51 |
| control | | | | | |
| Ef1a negative | N/A | EF1a | 1.11 | 0.38 | 0.29 |
| control | | | | | |
| Uninsulated | N/A | Uninsulated | 100.05 | 100.21 | 55.84 |
| construct^{#} | | | | | |
| cHS4 1200 bp | Forward | cHS4_1200 bp_fwd | 28.97 | 27.79 | 21.05 |
| cHS4 1200 bp | Reverse | cHS4_1200 bp_rev | 14.52 | 12.05 | 8.89 |
| cHS4 250 bp | Forward | cHS4_250 bp_fwd | 24.78 | 23.62 | 21.74 |
| cHS4 250 bp | Reverse | cHS4_250 bp_rev | 45.20 | 39.37 | 36.76 |
| cHS4 400 bp | Forward | cHS4_400 bp_fwd | 23.29 | 21.55 | 19.31 |
| (alternative) (SEQ | | | | | |
| ID NO: 69) | | | | | |
| cHS4 400 bp | Reverse | cHS4_400 bp_rev | 41.30 | 34.13 | 29.56 |
| (alternative) (SEQ | | | | | |
| ID NO: 70) | | | | | |
| cHS4 650 3SA bp | Forward | cHS4_650 bp_fwd | 33.81 | 32.04 | 25.90 |
| (SEQ ID NO: 28) | | | | | |
| cHS4 650 3SA bp | Reverse | cHS4_650 bp_rev | 27.48 | 30.66 | 28.12 |
| (SEQ ID NO: 56) | | | | | |
| A1 300 bp | Forward | A1_300 bp_fwd | 53.07 | 51.40 | 46.21 |
| A1 300 bp | Reverse | A1_300 bp_rev | 84.98 | 86.63 | 86.72 |
| A2 266 bp | Forward | A2_266 bp_fwd | 51.08 | 46.71 | 47.97 |
| A2 266 bp | Reverse | A2_266 bp_rev | 26.87 | 24.34 | 26.35 |
| C1 325 bp | Forward | C1_325 bp_fwd | 21.04 | 17.35 | 18.81 |
| C1 325 bp | Reverse | C1_325 bp_rev | 65.54 | 73.39 | 71.25 |
| B4 260 bp | Forward | B4_260 bp_fwd | 84.83 | 77.32 | 72.71 |
| B4 260 bp | Reverse | B4_260 bp_rev | 86.86 | 81.75 | 78.17 |
| 22-3 238 bp | Forward | 22-3_238 bp_fwd | 12.80 | 9.49 | 10.13 |
| 22-3 238 bp | Reverse | 22-3_238 bp_rev | 57.55 | 52.82 | 50.66 |
| FB 77 bp | Forward | FB_77 bp_fwd | 38.00 | 36.51 | 34.84 |
| FB 77 bp | Reverse | FB_77 bp_rev | 53.57 | 47.30 | 47.03 |
| Foamy Virus (FV) | Forward | FV_36 bp_fwd | 38.17 | 68.68 | 65.44 |
| 36 bp | | | | | |
| Foamy Virus (FV) | Reverse | FV_36 bp_rev | 27.27 | 24.66 | 25.52 |
| 36 bp | | | | | |
| Sns5 458 bp | Forward | Sns5_458 bp_fwd | 81.61 | 79.13 | 56.61 |
| Sns5 458 bp | Reverse | Sns5_458 bp_rev | 71.21 | 61.56 | 100.00 |

*relative to uninsulated construct^{#}

### REFERENCES

Goodman et aL, 2018, Foamy Virus Vector Carries a Strong Insulator in Its Long Terminal Repeat Which Reduces Its Genotoxic Potential′, J. of ViroL, 92 (1): e01639-17.
Livak and Schmittgen, 2001, ‘Analysis of Relative Gene Expression Data Using Real-Time Quantitative PCR and the 2-44CT Method’, Methods, Vol 25, pp. 402-408.
Ryu et aL, 2007, ‘An experimental system for the evaluation of retroviral vector design to 5 diminish the risk for proto-oncogene activation’, Blood, 111 (4): 1866-75.
Zhou et aL, 2010, ‘A self-inactivating lentiviral vector for SCID-X1 gene therapy that does not activate LMO2 expression in human T cells’, Blood, 116 (6): 900-908.

Sequences of the Disclosure

| SEQ | |
|---|---|
| ID | |
| NO: | Sequence |
| 1 | gacggatcgggagatctcccgatcccctatggtgcactctcagtacaatctgctctgatgccgcatagttaagccagtatctgctccct |
| | gcttgtgtgttggaggtcgctgagtagtgcgcgagcaaaatttaagctacaacaaggcaaggcttgaccgacaattgcatgaagaatct |
| | gcttagggttaggcgttttgcgctgcttcgcgatgtacgggccagatatacgcgtcttaagctggagtacggtggcatgatcttggctc |
| | actgcaacctctgcctcctgggttcaagcaattctcctgcctccttagtagctgggattacaggcgcctgccatcacgcccagctaatt |
| | tttatatttttagtagagacggggtttcaccatgttggtcaggctggtctcaaactcctgacctcaagtgatctgcccaccttggcctc |
| | ccaaagtgttgggattataggcctgtgccaccgcagctggccagacttgttatatggtgtcaagatgctccaagaatgagtgttctagc |
| | agtgcaaggtgaatgctatgtggccttttatgtcctatccttggaactcatatattccttcacatctcttttactctattctttaagac |
| | agtcacaaacctgcccagtttcaagggggagggatgtagacctcatttcttgagcagacaattattaaagaatttgcagccatgtttta |
| | aggctgccatatcttaaatgtatctatccatttttcccagttgctaagatcctttattttgtgtaagaatcttttcatggcaaattaaa |
| | aaaacaactcatgctagcctgggggaaaaaaggcatggataatttattataaggatgcagtgttatctcacagaacactcccaaaaatg |
| | tcacttaagtcctaatgtgtgctcagcgccagtgggtgctgaggggttagtgaggccaggtcttccctgctgatgcatcagcacacctg |
| | ggcagtgatggaattctcgagtggtttgtctggtcaaccaccgcggtctcagtggtgtacggtacaaacccactcgagtacgtatcgat |
| | gaacagagagacagcagaatatgggccaaacaggatatctgtggtaagcagttcctgccccggctcagggccaagaacagttggaacag |
| | cagaatatgggccaaacaggatatctgtggtaagcagttcctgccccggctcagggccaagaacagatggtccccagatgcggtcccgc |
| | cctcagcagtttctagagaaccatcagatgtttccagggtgccccaaggacctgaaatgaccctgtgccttatttgaactaaccaatca |
| | gttcgcttctcgcttctgttcgcgcgcttctgctccccgagctctatataagcagagctcgtttagtgaaccgtcagatcggcgcgcca |
| | attcaagcgagaagacaagggcagcctgcaggctagccaccatggtgagcaagggcgaggagctgttcaccggggtggtgcccatcctg |
| | gtcgagctggacggcgacgtaaacggccacaagttcagcgtgtccggcgagggcgagggcgatgccacctacggcaagctgaccctgaa |
| | gttcatctgcaccaccggcaagctgcccgtgccctggcccaccctcgtgaccaccctgacctacggcgtgcagtgcttcagccgctacc |
| | ccgaccacatgaagcagcacgacttcttcaagtccgccatgcccgaaggctacgtccaggagcgcaccatcttcttcaaggacgacggc |
| | aactacaagacccgcgccgaggtgaagttcgagggcgacaccctggtgaaccgcatcgagctgaagggcatcgacttcaaggaggacgg |
| | caacatcctggggcacaagctggagtacaactacaacagccacaacgtctatatcatggccgacaagcagaagaacggcatcaaggtga |
| | acttcaagatccgccacaacatcgaggacggcagcgtgcagctcgccgaccactaccagcagaacacccccatcggcgacggccccgtg |
| | ctgctgcccgacaaccactacctgagcacccagtccgccctgagcaaagaccccaacgagaagcgcgatcacatggtcctgctggagtt |
| | cgtgaccgccgccgggatcactctcggcatggacgagctgtacaagGGCTCCGGAGCTACTAACTTCAGCCTGCTGAAGCAGGCTGGAG |
| | ACGTGGAGGAGAACCCTGGACCTATGACCGAGTACAAGCCCACGGTGCGCCTCGCCACCCGCGACGACGTCCCCAGGGCCGTGCGCACC |
| | CTCGCCGCCGCGTTCGCCGACTACCCCGCCACGCGCCACACCGTCGATCCGGACCGCCACATCGAGCGGGTCACCGAGCTGCAAGAACT |
| | CTTCCTCACGCGCGTCGGGCTCGACATCGGCAAGGTGTGGGTCGCGGACGACGGCGCCGCGGTGGCGGTCTGGACCACGCCGGAGAGCG |
| | TCGAAGCGGGGGCGGTGTTCGCCGAGATCGGCCCGCGCATGGCCGAGTTGAGCGGTTCCCGGCTGGCCGCGCAGCAACAGATGGAAGGC |
| | CTCCTGGCGCCGCACCGGCCCAAGGAGCCCGCGTGGTTCCTGGCCACCGTCGGCGTCTCGCCCGACCACCAGGGCAAGGGTCTGGGCAG |
| | CGCCGTCGTGCTCCCCGGAGTGGAGGCGGCCGAGCGCGCCGGGGTGCCCGCCTTCCTGGAAACCTCCGCGCCCCGCAACCTCCCCTTCT |
| | ACGAGCGGCTCGGCTTCACCGTCACCGCCGACGTCGAGGTGCCCGAAGGACCGCGCACCTGGTGCATGACCCGCAAGCCCGGTGCCGGA |
| | AGCGGAGAGGGCAGAGGAAGTCTGCTAACATGCGGTGACGTCGAGGAGAATCCTGGACCTatggcctcctaccctggccatcagcacgc |
| | ctccgccttcgaccaggccgccaggtccagaggccactccaaccggcggaccgccctgcggcctcggagacagcaggaggccaccgaag |
| | tccggcctgagcagaagatgcctaccctgctgcgggtgtacatcgacggccctcacggcatgggcaagaccaccaccacccagctgctg |
| | gtcgccctgggctcccgggacgacatcgtgtacgtgcctgagcctatgacctactggcgggtgctgggcgcctccgagacaatcgccaa |
| | catctataccacccagcaccggctggaccagggcgagatctctgccggcgacgccgccgtggtgatgacctccgcccagatcaccatgg |
| | gcatgccttacgccgtgaccgacgccgtgctggcccctcacatcggaggcgaggccggatcttctcacgcccctccccctgccctgacc |
| | ctgatcttcgaccggcaccctatcgccgccctgctgtgctaccctgccgccagatacctgatgggctccatgacccctcaggctgtgct |
| | ggccttcgtggccctgatccctcccaccctgcctggcaccaacatcgtgctgggagccctgcctgaggaccggcacatcgaccggctgg |
| | ccaagaggcagcggcctggcgagaggctggacctggccatgctggccgccatccggcgggtgtacggcctgctggccaacaccgtgcgg |
| | tatctgcagtgcggcggctcctggagagaggactggggccagctgtccggcacagccgtgccacctcagggcgccgagcctcagtccaa |
| | cgctggccctagaccacacatcggcgacaccctgtttaccctgttcagagcccctgagctgctggcccccaacggcgacctgtacaacg |
| | tgttcgcctgggctctggacgtgctggccaagcggctgagatccatgcacgtgttcatcctggactacgaccagtcccctgccggatgc |
| | agagatgccctgctgcagctgacctccggcatggtgcagacccacgtgaccacccctggctccatccctaccatctgcgacctggcccg |
| | gaccttcgcccgggagatgggcgaggccaactaactagttcgaatcaacctctggattacaaaatttgtgaaagattgactggtattct |
| | taactatgttgctccttttacgctatgtggatacgctgctttaatgcctttgtatcatgctattgcttcccgtatggctttcattttct |
| | cctccttgtataaatcctggttgctgtctctttatgaggagttgtggcccgttgtcaggcaacgtggcgtggtgtgcactgtgtttgct |
| | gacgcaacccccactggttggggcattgccaccacctgtcagctcctttccgggactttcgctttccccctccctattgccacggcgga |
| | actcatcgccgcctgccttgcccgctgctggacaggggctcggctgttgggcactgacaattccgtggtgttgtcggggaaatcatcgt |
| | cctttccttggctgttcgcctgtgttgccacctggattctgcgcgggacgtccttctgctacgtcccttcggccctcaatccagcggac |
| | cttccttcccgcggcctgctgccggctctgcggcctcttccgcgtcttcgccttcgccctcagacgagtcggatctccctttgggccgc |
| | ctccccgcagggcccgtttaaactgtgccttctagttgccagccatctgttgtttgcccctcccccgtgccttccttgaccctggaagg |
| | tgccactcccactgtcctttcctaataaaatgaggaaattgcatcgcattgtctgagtaggtgtcattctattctggggggggggtggg |
| | gcaggacagcaagggggaggattgggaagacaatagcaggcatgctggggatgcggtgggctctatggatccgttaacaccggtgtggt |
| | ttgtctggtcaaccaccgcggactcagtggtgtacggtacaaacccaccggtaccctattgctccagaggcccttaggtgatgccacta |
| | atgggagggactggagaactgtggcaggcctgtcacagagcaactgaagaggccacagtatggagctgcttgtgccgctcccaggtggg |
| | gactggaaagctggagatctagggagggctgtttggagcagtgttcttccatccctacacattccatccccacagaaggaattcccatt |
| | tctttctagagggtaagcttctcagatctttcttcctcttctgtcaggctgcctctggccatcccagcctagtagatttagtcatccat |
| | tggtttcttgagtgtgccttgcattgtgacagtgaacaagacattcaagatcccgagaagatggaaaaatgctctgacctgggatccag |
| | cagccctgaccctgtctgttactctccaagtgactttggacatatagtcaatcctctccctctgggcctcagtttccccatatgtcaaa |
| | tcagggccttgaccttgaaggatctctaagatcccttcagaactcccagaagaaagatgattctggcatattgagaactcctaaaattt |
| | caatcctcgacctcaagtttattttctgtcttctgccttactgctggctggcttactcttgctgtaatttatttaactgagccgtgctg |
| | agcagtttagaactgagtttgcatgaatcactgtccataaaatcgagttaagtggatttcattccacacggcgcacctgtgtatataca |
| | caggaagcttcgtacggcggccgcacatgtgagcaaaaggccagcaaaaggccaggaaccgtaaaaaggccgcgttgctggcgtttttc |
| | cataggctccgcccccctgacgagcatcacaaaaatcgacgctcaagtcagaggtggcgaaacccgacaggactataaagataccaggc |
| | gtttccccctggaagctccctcgtgcgctctcctgttccgaccctgccgcttaccggatacctgtccgcctttctcccttcgggaagcg |
| | tggcgctttctcatagctcacgctgtaggtatctcagttcggtgtaggtcgttcgctccaagctgggctgtgtgcacgaaccccccgtt |
| | cagcccgaccgctgcgccttatccggtaactatcgtcttgagtccaacccggtaagacacgacttatcgccactggcagcagccactgg |
| | taacaggattagcagagcgaggtatgtaggcggtgctacagagttcttgaagtggtggcctaactacggctacactagaagaacagtat |
| | ttggtatctgcgctctgctgaagccagttaccttcggaaaaagagttggtagctcttgatccggcaaacaaaccaccgctggtagcggt |
| | ggtttttttgtttgcaagcagcagattacgcgcagaaaaaaaggatctcaagaagatcctttgatcttttctacggggtctgacgctca |
| | gtggaacgaaaactcacgttaagggattttggtcatgagattatcaaaaaggatcttcacctagatccttttaaattaaaaatgaagtt |
| | ttaaatcaatctaaagtatatatgagtaaacttggtctgacagttaccaatgcttaatcagtgaggcacctatctcagcgatctgtcta |
| | tttcgttcatccatagttgcctgactccccgtcgtgtagataactacgatacgggagggcttaccatctggccccagtgctgcaatgat |
| | accgcgagacccacgctcaccggctccagatttatcagcaataaaccagccagccggaagggccgagcgcagaagtggtcctgcaactt |
| | tatccgcctccatccagtctattaattgttgccgggaagctagagtaagtagttcgccagttaatagtttgcgcaacgttgttgccatt |
| | gctacaggcatcgtggtgtcacgctcgtcgtttggtatggcttcattcagctccggttcccaacgatcaaggcgagttacatgatcccc |
| | catgttgtgcaaaaaagcggttagctccttcggtcctccgatcgttgtcagaagtaagttggccgcagtgttatcactcatggttatgg |
| | cagcactgcataattctcttactgtcatgccatccgtaagatgcttttctgtgactggtgagtactcaaccaagtcattctgagaatag |
| | tgtatgcggcgaccgagttgctcttgcccggcgtcaatacgggataataccgcgccacatagcagaactttaaaagtgctcatcattgg |
| | aaaacgttcttcggggcgaaaactctcaaggatcttaccgctgttgagatccagttcgatgtaacccactcgtgcacccaactgatctt |
| | cagcatcttttactttcaccagcgtttctgggtgagcaaaaacaggaaggcaaaatgccgcaaaaaagggaataagggcgacacggaaa |
| | tgttgaatactcatactcttcctttttcaatattattgaagcatttatcagggttattgtctcatgagcggatacatatttgaatgtat |
| | ttagaaaaataaacaaataggggttccgcgcacatttccccgaaaagtgccacctgacgtc |
| 2 | gacggatcgggagatctcccgatcccctatggtgcactctcagtacaatctgctctgatgccgcatagttaagccagtatctgctccct |
| | gcttgtgtgttggaggtcgctgagtagtgcgcgagcaaaatttaagctacaacaaggcaaggcttgaccgacaattgcatgaagaatct |
| | gcttagggttaggcgttttgcgctgcttcgcgatgtacgggccagatatacgcgtcttaagctccaggttcttgccgtaatgtttcagc |
| | ttaaagaaaaccaacccatacttctttaggttgccctgaaaaggtgaatcttctgtgaagccttttctgtctttatctgttaaaattaa |
| | ttaagctcttgtttgtgctcccagaaaatcactttagaccttagaaagatggcagcctcagttttctcatttgtaatataggggtaccc |
| | atagggtcattgtgagaaataatagaaataaatcatggaagtgcttggatagtagctggtacttagccctcatcagtcatcattgtcat |
| | tgttgttaagagtaataataataataatatcacttattataattactcacctctaggtttttaaaaaacttacaggccgggcacattgg |
| | ctaacgcctgtaatcttagcactttgggaggccaaggcaggcagatcgcctgaggtcagcagttcaagaccagcttgggcaacatggtg |
| | aaaccctgtctctactaaaaatatgaaaattagccacgcgtggtggctcatgcctgtaatcccagctactcgggaggctgagagaggag |
| | aattgcttgaacccgggaggcggaggttgcagtgagccaagatggcactattgcactccagcctgggtgacgagaaactccatctcata |
| | aaaacaaaaacaaaaacaaaaaaacttacataaatttatggggaacaagtacaattttgtgacaagcgtaaattgcatagtcgtgaagt |
| | cagggcttctgaattctcgagtggtttgtctggtcaaccaccgcggtctcagtggtgtacggtacaaacccactcgagtacgtatcgat |
| | gaacagagagacagcagaatatgggccaaacaggatatctgtggtaagcagttcctgccccggctcagggccaagaacagttggaacag |
| | cagaatatgggccaaacaggatatctgtggtaagcagttcctgccccggctcagggccaagaacagatggtccccagatgcggtcccgc |
| | cctcagcagtttctagagaaccatcagatgtttccagggtgccccaaggacctgaaatgaccctgtgccttatttgaactaaccaatca |
| | gttcgcttctcgcttctgttcgcgcgcttctgctccccgagctctatataagcagagctcgtttagtgaaccgtcagatcggcgcgcca |
| | attcaagcgagaagacaagggcagcctgcaggctagccaccatggtgagcaagggcgaggagctgttcaccggggggtgcccatcctgg |
| | tcgagctggacggcgacgtaaacggccacaagttcagcgtgtccggcgagggcgagggcgatgccacctacggcaagctgaccctgaag |
| | ttcatctgcaccaccggcaagctgcccgtgccctggcccaccctcgtgaccaccctgacctacggcgtgcagtgcttcagccgctaccc |
| | cgaccacatgaagcagcacgacttcttcaagtccgccatgcccgaaggctacgtccaggagcgcaccatcttcttcaaggacgacggca |
| | actacaagacccgcgccgaggtgaagttcgagggcgacaccctggtgaaccgcatcgagctgaagggcatcgacttcaaggaggacggc |
| | aacatcctggggcacaagctggagtacaactacaacagccacaacgtctatatcatggccgacaagcagaagaacggcatcaaggtgaa |
| | cttcaagatccgccacaacatcgaggacggcagcgtgcagctcgccgaccactaccagcagaacacccccatcggcgacggccccgtgc |
| | tgctgcccgacaaccactacctgagcacccagtccgccctgagcaaagaccccaacgagaagcgcgatcacatggtcctgctggagttc |
| | gtgaccgccgccgggatcactctcggcatggacgagctgtacaagGGCTCCGGAGCTACTAACTTCAGCCTGCTGAAGCAGGCTGGAGA |
| | CGTGGAGGAGAACCCTGGACCTATGACCGAGTACAAGCCCACGGTGCGCCTCGCCACCCGCGACGACGTCCCCAGGGCCGTGCGCACCC |
| | TCGCCGCCGCGTTCGCCGACTACCCCGCCACGCGCCACACCGTCGATCCGGACCGCCACATCGAGCGGGTCACCGAGCTGCAAGAACTC |
| | TTCCTCACGCGCGTCGGGCTCGACATCGGCAAGGTGTGGGTCGCGGACGACGGCGCCGCGGTGGCGGTCTGGACCACGCCGGAGAGCGT |
| | CGAAGCGGGGGCGGTGTTCGCCGAGATCGGCCCGCGCATGGCCGAGTTGAGCGGTTCCCGGCTGGCCGCGCAGCAACAGATGGAAGGCC |
| | TCCTGGCGCCGCACCGGCCCAAGGAGCCCGCGTGGTTCCTGGCCACCGTCGGCGTCTCGCCCGACCACCAGGGCAAGGGTCTGGGCAGC |
| | GCCGTCGTGCTCCCCGGAGTGGAGGCGGCCGAGCGCGCCGGGGTGCCCGCCTTCCTGGAAACCTCCGCGCCCCGCAACCTCCCCTTCTA |
| | CGAGCGGCTCGGCTTCACCGTCACCGCCGACGTCGAGGTGCCCGAAGGACCGCGCACCTGGTGCATGACCCGCAAGCCCGGTGCCGGAA |
| | GCGGAGAGGGCAGAGGAAGTCTGCTAACATGCGGTGACGTCGAGGAGAATCCTGGACCTatggcctcctaccctggccatcagcacgcc |
| | tccgccttcgaccaggccgccaggtccagaggccactccaaccggcggaccgccctgcggcctcggagacagcaggaggccaccgaagt |
| | ccggcctgagcagaagatgcctaccctgctgcgggtgtacatcgacggccctcacggcatgggcaagaccaccaccacccagctgctgg |
| | tcgccctgggctcccgggacgacatcgtgtacgtgcctgagcctatgacctactggcgggtgctgggcgcctccgagacaatcgccaac |
| | atctataccacccagcaccggctggaccagggcgagatctctgccggcgacgccgccgtggtgatgacctccgcccagatcaccatggg |
| | catgccttacgccgtgaccgacgccgtgctggcccctcacatcggaggcgaggccggatcttctcacgcccctccccctgccctgaccc |
| | tgatcttcgaccggcaccctatcgccgccctgctgtgctaccctgccgccagatacctgatgggctccatgacccctcaggctgtgctg |
| | gccttcgtggccctgatccctcccaccctgcctggcaccaacatcgtgctgggagccctgcctgaggaccggcacatcgaccggctggc |
| | caagaggcagcggcctggcgagaggctggacctggccatgctggccgccatccggcgggtgtacggcctgctggccaacaccgtgcggt |
| | atctgcagtgcggcggctcctggagagaggactggggccagctgtccggcacagccgtgccacctcaggggccgagcctcagtccaacg |
| | ctggccctagaccacacatcggcgacaccctgtttaccctgttcagagcccctgagctgctggcccccaacggcgacctgtacaacgtg |
| | ttcgcctgggctctggacgtgctggccaagcggctgagatccatgcacgtgttcatcctggactacgaccagtcccctgccggatgcag |
| | agatgccctgctgcagctgacctccggcatggtgcagacccacgtgaccacccctggctccatccctaccatctgcgacctggcccgga |
| | ccttcgcccgggagatgggcgaggccaactaactagttcgaatcaacctctggattacaaaatttgtgaaagattgactggtattctta |
| | actatgttgctccttttacgctatgtggatacgctgctttaatgcctttgtatcatgctattgcttcccgtatggctttcattttctcc |
| | tccttgtataaatcctggttgctgtctctttatgaggagttgtggcccgttgtcaggcaacgtggcgtggtgtgcactgtgtttgctga |
| | cgcaacccccactggttggggcattgccaccacctgtcagctcctttccgggactttcgctttccccctccctattgccacggcggaac |
| | tcatcgccgcctgccttgcccgctgctggacaggggctcggctgttgggcactgacaattccgtggtgttgtcggggaaatcatcgtcc |
| | tttccttggctgttcgcctgtgttgccacctggattctgcgcgggacgtccttctgctacgtcccttcggccctcaatccagcggacct |
| | tccttcccgcggcctgctgccggctctgcggcctcttccgcgtcttcgccttcgccctcagacgagtcggatctccctttgggccgcct |
| | ccccgcagggcccgtttaaactgtgccttctagttgccagccatctgttgtttgcccctcccccgtgccttccttgaccctggaaggtg |
| | ccactcccactgtcctttcctaataaaatgaggaaattgcatcgcattgtctgagtaggtgtcattctattctggggggtggggggggc |
| | aggacagcaagggggaggattgggaagacaatagcaggcatgctggggatgcggtgggctctatggatccgttaacaccggtgtggttt |
| | gtctggtcaaccaccgcggactcagtggtgtacggtacaaacccaccggtaccaaggtatctatcaccagattgatatctattggtatc |
| | tatcaccagattgatatctattgatatctatcaccagattgatatcaccacgttgtacctattaattaatttctcatcatcctcccccc |
| | acccttctgagtctccagtgtatattattccataccctaatgccatgtgttcacattatttagctcccacttatgagtgaacaatgtga |
| | tatttgtctttcagtgtctgacttttttaacttaagataacagcttccagttctacccatgtggctgcgagagacatgatttcattctt |
| | ttttatggctgaataatattccattgcatatatatacaccacagttttaaatccaatcatccattgatggacacttaggttgattccac |
| | atctttgctattgtgactagtgctacgataaacatacaagtgcaggtatctaattatttcttttcctttggatggatacccagtagtgg |
| | gattgctggattgaatggtagttctaattttagttctctgacaaatctccatactgttcttcctagagattgtactaatttacattccc |
| | accaacagcgtataagagtttccttttctccacatcgccactaggttgtaagttccttgaatgtagggatcatgtctcttttcttcatt |
| | gttttataacaagagcctaggagtgtttggcacatggcaagtacttgttttctagctgacagatgaatgaacaaatgagtgaaggatgc |
| | ttaagcttcgtacggcggccgcacatgtgagcaaaaggccagcaaaaggccaggaaccgtaaaaaggccgcgttgctggcgtttttcca |
| | taggctccgcccccctgacgagcatcacaaaaatcgacgctcaagtcagaggtggcgaaacccgacaggactataaagataccaggcgt |
| | ttccccctggaagctccctcgtgcgctctcctgttccgaccctgccgcttaccggatacctgtccgcctttctcccttcgggaagcgtg |
| | gcgctttctcatagctcacgctgtaggtatctcagttcggtgtaggtcgttcgctccaagctgggctgtgtgcacgaaccccccgttca |
| | gcccgaccgctgcgccttatccggtaactatcgtcttgagtccaacccggtaagacacgacttatcgccactggcagcagccactggta |
| | acaggattagcagagcgaggtatgtaggcggtgctacagagttcttgaagtggtggcctaactacggctacactagaagaacagtattt |
| | ggtatctgcgctctgctgaagccagttaccttcggaaaaagagttggtagctcttgatccggcaaacaaaccaccgctggtagcggtgg |
| | tttttttgtttgcaagcagcagattacgcgcagaaaaaaaggatctcaagaagatcctttgatcttttctacggggtctgacgctcagt |
| | ggaacgaaaactcacgttaagggattttggtcatgagattatcaaaaaggatcttcacctagatccttttaaattaaaaatgaagtttt |
| | aaatcaatctaaagtatatatgagtaaacttggtctgacagttaccaatgcttaatcagtgaggcacctatctcagcgatctgtctatt |
| | tcgttcatccatagttgcctgactccccgtgtgtagataactacgatacgggagggcttaccatctggccccagtgctgcaatgatacc |
| | gcgagacccacgctcaccggctccagatttatcagcaataaaccagccagccggaagggccgagcgcagaagtggtcctgcaactttat |
| | ccgcctccatccagtctattaattgttgccgggaagctagagtaagtagttcgccagttaatagtttgcgcaacgttgttgccattgct |
| | acaggcatcgtggtgtcacgctcgtcgtttggtatggcttcattcagctccggttcccaacgatcaaggcgagttacatgatcccccat |
| | gttgtgcaaaaaagcggttagctccttcggtcctccgatcgttgtcagaagtaagttggccgcagtgttatcactcatggttatggcag |
| | cactgcataattctcttactgtcatgccatccgtaagatgcttttctgtgactggtgagtactcaaccaagtcattctgagaatagtgt |
| | atgcggcgaccgagttgctcttgcccggcgtcaatacgggataataccgcgccacatagcagaactttaaaagtgctcatcattggaaa |
| | acgttcttcggggcgaaaactctcaaggatcttaccgctgttgagatccagttcgatgtaacccactcgtgcacccaactgatcttcag |
| | catcttttactttcaccagcgtttctgggtgagcaaaaacaggaaggcaaaatgccgcaaaaaagggaataagggcgacacggaaatgt |
| | tgaatactcatactcttcctttttcaatattattgaagcatttatcagggttattgtctcatgagcggatacatatttgaatgtattta |
| | gaaaaataaacaaataggggttccgcgcacatttccccgaaaagtgccacctgacgtc |
| 3 | gacattgattattgactagttattaatagtaatcaattacggggtcattagttcatagcccatatatggagttccgcgttacataactt |
| | acggtaaatggcccgcctggctgaccgcccaacgacccccgcccattgacgtcaataatgacgtatgttcccatagtaacgccaatagg |
| | gactttccattgacgtcaatgggtggagtatttacggtaaactgcccacttggcagtacatcaagtgtatcatatgccaagtacgcccc |
| | ctattgacgtcaatgacggtaaatggcccgcctggcattatgcccagtacatgaccttatgggactttcctacttggcagtacatctac |
| | gtattagtcatcgctattaccatg |
| 4 | gtgatgcggttttggcagtacatcaatgggcgtggatagcggtttgactcacggggatttccaagtctccaccccattgacgtcaatgg |
| | gagtttgttttggcaccaaaatcaacgggactttccaaaatgtcgtaacaactccgccccattgacgcaaatgggcggtaggcgtgtac |
| | ggtgggaggtctatataagcagagct |
| 5 | gaacagagagacagcagaatatgggccaaacaggatatctgtggtaagcagttcctgccccggctcagggccaagaacagttggaacag |
| | cagaatatgggccaaacaggatatctgtggtaagcagttcctgccccggctcagggccaagaacagatggtccccagatgcggtcccgc |
| | cctcagcagtttctagagaaccatcagatgtttccagggtgccccaaggacctgaaatgaccctgtgccttatttgaactaaccaatca |
| | gttcgcttctcgcttctgttcgcgcgcttctgctccccgagctctatataagcagagctcgtttagtgaaccgtcagatc |
| 6 | atggcctcctaccctggccatcagcacgcctccgccttcgaccaggccgccaggtccagaggccactccaaccggcggaccgccctgcg |
| | gcctcggagacagcaggaggccaccgaagtccggcctgagcagaagatgcctaccctgctgcgggtgtacatcgacggccctcacggca |
| | tgggcaagaccaccaccacccagctgctggtcgccctgggctcccgggacgacatgtgtacgtgcctgagcctatgacctactggcggg |
| | tgctgggcgcctccgagacaatcgccaacatctataccacccagcaccggctggaccagggcgagatctctgccggcgacgccgccgtg |
| | gtgatgacctccgcccagatcaccatgggcatgccttacgccgtgaccgacgccgtgctggcccctcacatcggaggcgaggccggatc |
| | ttctcacgcccctccccctgccctgaccctgatcttcgaccggcaccctatcgccgccctgctgtgctaccctgccgccagatacctga |
| | tgggctccatgacccctcaggctgtgctggccttcgtggccctgatccctcccaccctgcctggcaccaacatcgtgctgggagccctg |
| | cctgaggaccggcacatcgaccggctggccaagaggcagcggcctggcgagaggctggacctggccatgctggccgccatccggcgggt |
| | gtacggcctgctggccaacaccgtgcggtatctgcagtgcggcggctcctggagagaggactggggccagctgtccggcacagccgtgc |
| | cacctcagggcgccgagcctcagtccaacgctggccctagaccacacatcggcgacaccctgtttaccctgttcagagcccctgagctg |
| | ctggcccccaacggcgacctgtacaacgtgttcgcctgggctctggacgtgctggccaagcggctgagatccatgcacgtgttcatcct |
| | ggactacgaccagtcccctgccggatgcagagatgccctgctgcagctgacctccggcatggtgcagacccacgtgaccacccctggct |
| | ccatccctaccatctgcgacctggcccggaccttcgcccgggagatgggcgaggccaactaa |
| 7 | atggtgagcaagggcgaggagctgttcaccggggtggtgcccatcctggtcgagctggacggcgacgtaaacggccacaagttcagcgt |
| | gtccggcgagggcgagggcgatgccacctacggcaagctgaccctgaagttcatctgcaccaccggcaagctgcccgtgccctggccca |
| | ccctcgtgaccaccctgacctacggcgtgcagtgcttcagccgctaccccgaccacatgaagcagcacgacttcttcaagtccgccatg |
| | cccgaaggctacgtccaggagcgcaccatcttcttcaaggacgacggcaactacaagacccgcgccgaggtgaagttcgagggcgacac |
| | cctggtgaaccgcatcgagctgaagggcatcgacttcaaggaggacggcaacatcctggggcacaagctggagtacaactacaacagcc |
| | acaacgtctatatcatggccgacaagcagaagaacggcatcaaggtgaacttcaagatccgccacaacatcgaggacggcagcgtgcag |
| | ctcgccgaccactaccagcagaacacccccatcggcgacggccccgtgctgctgcccgacaaccactacctgagcacccagtccgccct |
| | gagcaaagaccccaacgagaagcgcgatcacatggtcctgctggagttcgtgaccgccgccgggatcactctcggcatggacgagctgt |
| | acaag |
| 8 | atgaccgagtacaagcccacggtgcgcctcgccacccgcgacgacgtccccagggccgtgcgcaccctcgccgccgcgttcgccgacta |
| | ccccgccacgcgccacaccgtcgatccggaccgccacatcgagcgggtcaccgagctgcaagaactcttcctcacgcgcgtcgggctcg |
| | acatcggcaaggtgtgggtcgcggacgacggcgccgcggtggcggtctggaccacgccggagagcgtcgaagcgggggcggtgttcgcc |
| | gagatcggcccgcgcatggccgagttgagcggttcccggctggccgcgcagcaacagatggaaggcctcctggcgccgcaccggcccaa |
| | ggagcccgcgtggttcctggccaccgtcggcgtctcgcccgaccaccagggcaagggtctgggcagcgccgtcgtgctccccggagtgg |
| | aggcggccgagcgcgccggggtgcccgccttcctggaaacctccgcgccccgcaacctccccttctacgagcggctcggcttcaccgtc |
| | accgccgacgtcgaggtgcccgaaggaccgcgcacctggtgcatgacccgcaagcccggtgcc |
| 9 | GSGATNFSLLKQAGDVCCNPGP |
| 10 | gctactaacttcagcctgctgaagcaggctggagacgtggaggagaaccctggacct |
| 11 | ggaagcggagctactaacttcagcctgctgaagcaggctggagacgtggaggagaaccctggacct |
| 12 | ggcagcggcgccacaaacttctctctgctaaagcaagcaggtgatgttgaagaaaaccccgggcct |
| 13 | ggctccggcgagggcaggggaagtcttctaacatgcggggacgtggaggaaaatcccggccca |
| 14 | GSGCGRGSLLTCGDVCCNPGP |
| 15 | gagggcagaggaagtctgctaacatgcggtgacgtcgaggagaatcctggacct |
| 16 | ggaagcggagagggcagaggaagtctgctaacatgcggtgacgtcgaggagaatcctggacct |
| 17 | (c)tgtggaatgtgtgtcagttagggtgtggaaagtccccaggctccccagcaggcagaagtatgcaaagcatgcatctcaattagtca |
| | gcaaccaggtgtggaaagtccccaggctccccagcaggcagaagtatgcaaagcatgcatctcaattagtcagcaaccatagtcccgcc |
| | cctaactccgcccatcccgcccctaactccgcccagttccgcccattctccgccccatggctgactaattttttttatttatgcagagg |
| | ccgaggccgcctctgcctctgagctattccagaagtagtgaggaggcttttttggaggcctaggcttttgcaaaaagctc |
| 18 | aacttgtttattgcagcttataatggttacaaataaagcaatagcatcacaaatttcacaaataaagcatttttttcactgcattctag |
| | ttgtggtttgtccaaactcatcaatgtatcttatcatgtctg |
| 19 | ctgtgccttctagttgccagccatctgttgtttgcccctcccccgtgccttccttgaccctggaaggtgccactcccactgtcctttcc |
| | taataaaatgaggaaattgcatcgcattgtctgagtaggtgtcattctattctggggggtggggggggcaggacagcaagggggaggat |
| | tgggaagacaatagcaggcatgctggggatgcggtgggctctatgg |
| 20 | aatcaacctctggattacaaaatttgtgaaagattgactggtattcttaactatgttgctccttttacgctatgtggatacgctgcttt |
| | aatgcctttgtatcatgctattgcttcccgtatggctttcattttctcctccttgtataaatcctggttgctgtctctttatgaggagt |
| | tgtggcccgttgtcaggcaacgtggcgtggtgtgcactgtgtttgctgacgcaacccccactggttggggcattgccaccacctgtcag |
| | ctcctttccgggactttcgctttccccctccctattgccacggcggaactcatcgccgcctgccttgcccgctgctggacaggggctcg |
| | gctgttgggcactgacaattccgtggtgttgtcggggaaatcatcgtcctttccttggctgttcgcctgtgttgccacctggattctgc |
| | gcgggacgtccttctgctacgtcccttcggccctcaatccagcggaccttccttcccgcggcctgctgccggctctgcggcctcttccg |
| | cgtcttcgccttcgccctcagacgagtcggatctccctttgggccgcctccccgca |
| 21 | tcgtgaagtcagggcttcta |
| 22 | gagcaatagcatcactgccc |
| 23 | cagtgatgctattgctccag |
| 24 | ctccaggttcttgccgtaatgtttcagcttaaagaaaaccaacccatacttctttaggttgccctgaaaaggtgaatcttctgtgaagc |
| | cttttctgtctttatctgttaaaattaattaagctcttgtttgtgctcccagaaaatcactttagaccttagaaagatggcagcctcag |
| | ttttctcatttgtaatataggggtacccatagggtcattgtgagaaataatagaaataaatcatggaagtgcttggatagtagctggta |
| | cttagccctcatcagtcatcattgtcattgttgttaagagtaataataataataatatcacttattataattactcacctctaggtttt |
| | taaaaaacttacaggccgggcacattggctaacgcctgtaatcttagcactttgggaggccaaggcaggcagatcgcctgaggtcagca |
| | gttcaagaccagcttgggcaacatggtgaaaccctgtctctactaaaaatatgaaaattagccacgcgtggtggctcatgcctgtaatc |
| | ccagctactcgggaggctgagagaggagaattgcttgaacccgggaggcggaggttgcagtgagccaagatggcactattgcactccag |
| | cctgggtgacgagaaactccatctcataaaaacaaaaacaaaaacaaaaaaacttacataaatttatggggaacaagtacaattttgtg |
| | acaagcgtaaattgcatagtcgtgaagtcagggcttct |
| 25 | aaggtatctatcaccagattgatatctattggtatctatcaccagattgatatctattgatatctatcaccagattgatatcaccacgt |
| | tgtacctattaattaatttctcatcatcctccccccacccttctgagtctccagtgtatattattccataccctaatgccatgtgttca |
| | cattatttagctcccacttatgagtgaacaatgtgatatttgtctttcagtgtctgacttttttaacttaagataacagcttccagttc |
| | tacccatgtggctgcgagagacatgatttcattcttttttatggctgaataatattccattgcatatatatacaccacagttttaaatc |
| | caatcatccattgatggacacttaggttgattccacatctttgctattgtgactagtgctacgataaacatacaagtgcaggtatctaa |
| | ttatttcttttcctttggatggatacccagtagtgggattgctggattgaatggtagttctaattttagttctctgacaaatctccata |
| | ctgttcttcctagagattgtactaatttacattcccaccaacagcgtataagagtttccttttctccacatcgccactaggttgtaagt |
| | tccttgaatgtagggatcatgtctcttttcttcattgttttataacaagagcctaggagtgtttggcacatggcaagtacttgttttct |
| | agctgacagatgaatgaacaaatgagtgaaggatgctt |
| 26 | ctggagtacggtggcatgatcttggctcactgcaacctctgcctcctgggttcaagcaattctcctgcctccttagtagctgggattac |
| | aggcgcctgccatcacgcccagctaatttttatatttttagtagagacggggtttcaccatgttggtcaggctggtctcaaactcctga |
| | cctcaagtgatctgcccaccttggcctcccaaagtgttgggattataggcctgtgccaccgcagctggccagacttgttatatggtgtc |
| | aagatgctccaagaatgagtgttctagcagtgcaaggtgaatgctatgtggccttttatgtcctatccttggaactcatatattccttc |
| | acatctcttttactctattctttaagacagtcacaaacctgcccagtttcaagggggagggatgtagacctcatttcttgagcagacaa |
| | ttattaaagaatttgcagccatgttttaaggctgccatatcttaaatgtatctatccatttttcccagttgctaagatcctttattttg |
| | tgtaagaatcttttcatggcaaattaaaaaaacaactcatgctagcctgggggaaaaaaggcatggataatttattataaggatgcagt |
| | gttatctcacagaacactcccaaaaatgtcacttaagtcctaatgtgtgctcagcgccagtgggtgctgaggggttagtgaggccaggt |
| | cttccctgctgatgcatcagcacacctgggcagtgatg |
| 27 | ctattgctccagaggcccttaggtgatgccactaatgggagggactggagaactgtggcaggcctgtcacagagcaactgaagaggcca |
| | cagtatggagctgcttgtgccgctcccaggtggggactggaaagctggagatctagggagggctgtttggagcagtgttcttccatccc |
| | tacacattccatccccacagaaggaattcccatttctttctagagggtaagcttctcagatctttcttcctcttctgtcaggctgcctc |
| | tggccatcccagcctagtagatttagtcatccattggtttcttgagtgtgccttgcattgtgacagtgaacaagacattcaagatcccg |
| | agaagatggaaaaatgctctgacctgggatccagcagccctgaccctgtctgttactctccaagtgactttggacatatagtcaatcct |
| | ctccctctgggcctcagtttccccatatgtcaaatcagggccttgaccttgaaggatctctaagatcccttcagaactcccagaagaaa |
| | gatgattctggcatattgagaactcctaaaatttcaatcctcgacctcaagtttattttctgtcttctgccttactgctggctggctta |
| | ctcttgctgtaatttatttaactgagccgtgctgagcagtttagaactgagtttgcatgaatcactgtccataaaatcgagttaagtgg |
| | atttcattccacacggcgcacctgtgtatatacacagg |
| 28 | gagctcacggggacagcccccccccaaagcccccagggatgtaattacgtccctcccccgctagggggcagcagcgagccgcccggggc |
| | tccgctccggtccggcgctccccccgcatccccgagccggcagcgtgcggggacagcccgggcacggggaaggtggcacgggatcgctt |
| | tcctctgaacgcttctcgctgctctttgagccagcagacaccaggggggatacggggaaaaagcttgatatcatgtgtctgagcctgca |
| | tgtttgatggtgtcaggatgcaagcagaaggggtggaagagcttgcctggagagatacagctgggtcagtaggactgggacaggcagct |
| | ggagaattgccatgtagatgttcatacaatcgtcaaatcatgaaggctggaaaagccctccaagatccccaagaccaaccccaacccac |
| | ccaccgtgcccactggccatgtccctcagtgccacatccccacagttcttcatcacctccagggacggtgacccccccacctccgtggg |
| | cagctgtgccactgcagcaccgctctttggagaaggtaaatcttgctaaatccagcccgaccctcccctggcacaacgtaaggccatta |
| | tctctcatccaactccaggacggagtcagtgaggatggggct |
| 29 | gagctcacggggacagcccccccccaaagcccccagggatgtaattacgtccctcccccgctagggggcagcagcgagccgcccggggc |
| | tccgctccggtccggcgctccccccgcatccccgagccggcagcgtgcggggacagcccgggcacggggaaggtggcacgggatcgctt |
| | tcctctgaacgcttctcgctgctctttgagcctgcagacacctggggggatacggggaaaaagcttgatatcatgtgtctgagcctgca |
| | tgtttgatggtgtctggatgcaagcagaaggggtggaagagcttgcctggagagatacagctgggtcagtaggactgggacaggcagct |
| | ggagaattgccatgtagatgttcatacaatcgtcaaatcatgaaggctggaaaagccctccaagatccccaagaccaaccccaacccac |
| | ccaccgtgcccactggccatgtccctcagtgccacatccccacagttcttcatcacctccagggacggtgacccccccacctccgtggg |
| | cagctgtgccactgcagcaccgctctttggagaaggtaaatcttgctaaatccagcccgaccctcccctggcacaacgtaaggccatta |
| | tctctcatccaactccaggacggagtcagtgaggatggggct |
| 30 | agccccatcctcactgactccgtcctggagttggatgagagataatggccttacgttgtgccaggggagggtcgggctggatttagcaa |
| | gatttaccttctccaaagagcggtgctgcagtggcacagctgcccacggaggtgggggggtcaccgtccctggaggtgatgaagaactg |
| | tggggatgtggcactgagggacatggccagtgggcacggggggggttggggttggtcttggggatcttggagggcttttccagccttca |
| | tgatttgacgattgtatgaacatctacatggcaattctccagctgcctgtcccagtcctactgacccagctgtatctctccaggcaagc |
| | tcttccaccccttctgcttgcatccagacaccatcaaacatgcaggctcagacaca |
| 31 | aagctttttccccgtatccccccaggtgtctgcaggctcaaagagcagcgagaagcgttcagaggaaagcgatcccgtgccaccttccc |
| | cgtgcccgggctgtccccgcacgctgccggctcggggatgcggggggagcgccggaccggagcggagccccgggcggctcgctgctgcc |
| | ccctagcggggggggacgtaattacatccctgggggctttgggggggggctgtccccgtgagctc |
| 32 | ctggggggggagagaccaggggcaggtgaggaaaggcagggcccccagaatccctccatgcctgcccctcagtctccaggacttatgtg |
| | caggtaccgtttggagctgtggtgcagttcccagtctcaccaccagatggcaccatgcccctgcagaagcagtgcccagagcaggccag |
| | gtggttctcgggggctgcggtggaggaatccacccagccgaagctctggcagggaagg |
| 33 | ccaatcgtggcatatcctctaaactttcttttcccttcataaatcctctttcttttttttccccctcacagttttcctgaacaggttga |
| | ctattaattgtgtctgcttgatgtggacaccaggtggcgctggacatcagatttggagaggcagttgtctagggaaccgggctctgtgc |
| | cagcgcaggaggcaggctggctctcctattccagggatgctcatccaggaaggaaaggttgcatgctggacacactaaccttgaagaat |
| | tcttctgtctctctcgtcatttagaaaggaagg |
| 34 | agagcgagattccgtctcaaagaaaaaaaaagtaatgaaatgaataaaatgagtcctagagccagtaaatgtcgtaaatgtctcagcta |
| | gtcaggtagtaaaaggtctcaactaggcagtggcagagcaggattcaaattcagggctgttgtgatgcctccgcagactctgagcgcca |
| | cctggtggtaatttgtctgtgcctcttctgacgtggaagaacagcaactaacacactaacacggcatttactatgggccagccattgt |
| 35 | aagggagacatctagtgatataagtgtgaactacac |
| 36 | cccagggatgtacgtccctaacccgctagggggcagcacccaggcctgcactgccgcctgccggcaggggtccagtc |
| 37 | gcttcttggaggtgtgaccatcgctcgaggtggtgtactgcccaacatccaggctgtgctgcttcccaagaagaccggcaaatcaagct |
| | aaaggttttgcactcgcaaacctcaacacctcaacggcccttatcagggccaccaaatatacaagaaagaataaagtctctgtaattca |
| | taatagtctctgtaattcataataaactcctactgcaactacaactcagacgcaaccccctctcctcctcctcctccacccctcctccc |
| | ccccccctctctctctctctctctctctctctctgtcttccccattccggttttgtagttgttgtcgatcatccgatctaatatgccta |
| | tgcgaatacaatcacaaatgttattattataaacattctgttctgttctgttattgcttattgcttacacggatgaataaaagatactc |
| | tgtgtcgcacagt |
| 38 | gagctcacggggacagcccccccccaaagcccccagggatgtaattacgtccctcccccgctagggggcagcagcgagccgcccggggc |
| | tccgctccggtccggcgctccccccgcatccccgagccggcagcgtgcggggacagcccgggcacggggaaggtggcacgggatcgctt |
| | tcctctgaacgcttctcgctgctctttgagcctgcagacacctggggggatacggggaaaaagctttaggctgaaagagagatttagaa |
| | tgacagaatcatagaacggcctgggttgcaaaggagcacagtgctcatccagatccaaccccctgctatgtgcagggtcatcaaccagc |
| | agcccaggctgcccagagccacatccagcctggccttgaatgcctgcagggatggggcatccacagcctccttgggcaacctgttcagt |
| | gcgtcaccaccctctgggggaaaaactgcctcctcatatccaacccaaacctcccctgtctcagtgtaaagccattcccccttgtccta |
| | tcaagggggagtttgctgtgacattgttggtctggggtgacacatgtttgccaattcagtgcatcacggagaggcagatcttggggata |
| | aggaagtgcaggacagcatggacgtgggacatgcaggtgttgagggctctgggacactctccaagtcacagcgttcagaacagccttaa |
| | ggataagaagataggatagaaggacaaagagcaagttaaaacccagcatggagaggagcacaaaaaggccacagacactgctggtccct |
| | gtgtctgagcctgcatgtttgatggtgtctggatgcaagcagaaggggtggaagagcttgcctggagagatacagctgggtcagtagga |
| | ctgggacaggcagctggagaattgccatgtagatgttcatacaatcgtcaaatcatgaaggctggaaaagccctccaagatccccaaga |
| | ccaaccccaacccacccaccgtgcccactggccatgtccctcagtgccacatccccacagttcttcatcacctccagggacggtgaccc |
| | ccccacctccgtgggcagctgtgccactgcagcaccgctctttggagaaggtaaatcttgctaaatccagcccgaccctcccctggcac |
| | aacgtaaggccattatctctcatccaactccaggacggagtcagtgagaatatt |
| 39 | ggatccgaacagagagacagcagaatatgggccaaacaggatatctgtggtaagcagttcctgccccggctcagggccaagaacagttg |
| | gaacagcagaatatgggccaaacaggatatctgtggtaagcagttcctgccccggctcagggccaagaacagatggtccccagatgcgg |
| | tcccgccctcagcagtttctagagaaccatcagatgtttccagggtgccccaaggacctgaaatgaccctgtgccttatttgaactaac |
| | caatcagttcgcttctcgcttctgttcgcgcgcttctgctccccgagctctatataagcagagctcgtttagtgaaccgtcagatc |
| 40 | atggtgagcaagggcgaggccgtgatcaaggagttcatgaggttcaaggtgcacatggagggctccatgaacggtcacgagttcgagat |
| | cgagggcgaaggcgagggcaggccctacgagggcacccagaccgccaagctgaaggtgacaaagggggccccctgcccttctcctggga |
| | catcctgtcccctcagttcatgtacggctccagggccttcatcaagcaccccgccgacatccccgactactacaagcagtccttccccg |
| | agggcttcaagtgggagagggtgatgaacttcgaggacggcggcgccgtgaccgtgacccaggacacctccctggaggacggcaccctg |
| | atctacaaggtgaagctgaggggcaccaacttccctcctgacggccccgtgatgcagaagaagacaatgggctgggaggcctccaccga |
| | gaggctgtaccccgaggacggcgtgctgaagggcgacatcaagatggccctgaggctgaaggacggcggcagatacctggccgacttca |
| | agaccacctacaaggccaagaagcccgtgcagatgcccggcgcctacaacgtggacaggaagctggacatcacctcccacaacgaggac |
| | tacaccgtggtggagcagtacgagaggtccgagggcaggcactccaccggcggcatggacgagctgtataag |
| 41 | aatcaacctctggattacaaaatttgtgaaagattgactggtattcttaactatgttgctccttttacgctatgtggatacgctgcttt |
| | aatgcctttgtatcatgctattgcttcccgtatggctttcattttctcctccttgtataaatcctggttgctgtctctttatgaggagt |
| | tgtggcccgttgtcaggcaacgtggcgtggtgtgcactgtgtttgctgacgcaacccccactggttggggcattgccaccacctgtcag |
| | ctcctttccgggactttcgctttccccctccctattgccacggcggaactcatcgccgcctgccttgcccgctgctggacaggggctcg |
| | gctgttgggcactgacaattccgtggtgttgtcggggaaatcatcgtcctttccttggctgctcgcctgtgttgccacctggattctgc |
| | gcgggacgtccttctgctacgtcccttcggccctcaatccagcggaccttccttcccgcggcctgctgccggctctgcggcctcttccg |
| | cgtcttcgccttcgccctcagacgagtcggatctccctttgggccgcctccccgc |
| 42 | aggaggagatatgagggacaattggagaagtgaattatataaatataaagtagtaaaaattgaaccattaggagtagcacccaccaagg |
| | caaagagaagagtggtgcagagagaaaaaagagcagtgggaataggagctttgttccttgggttcttgggagcagcaggaagcactatg |
| | ggcgcagcgtcaatgacgctgacggtacaggccagacaattattgtctggtatagtgcagcagcagaacaatttgctgagggctattga |
| | ggcgcaacagcatctgttgcaactcacagtctggggcatcaagcagctccaggcaagaatcctggctgtggaaagatacctaaaggatc |
| | aacagctcctggggatttggggttgctctggaaaactcatttgcaccactgctgtgccttggaatgctagttggagtaataaatctctg |
| | gaacagatttggaatcacacgacctggatggagtgggacagagaaattaacaattacacaagcttaatacactccttaattgaagaatc |
| | gcaaaaccagcaagaaaagaatgaacaagaattattggaattagataaatgggcaagtttgtggaattggtttaacataacaaattggc |
| | tgtggtatataaaattattcataatgatagtaggaggcttggtaggtttaagaatagtttttgctgtactttctatagtgaatagagtt |
| | aggcagggatattcaccattatcgtttcagacccacctcccaaccccgaggggacc |
| 43 | aattccctacaatccccaaagtcaaggagtagtagaatctatgaataaagaattaaagaaaattataggacaggtaagagatcaggctg |
| | aacatcttaagacagcagtacaaatggcagtattcatccacaattttaaaagaaaaggggggattggggggtacagtgcaggggaaaga |
| | atagtagacataatagcaacagacatacaaactaaagaattacaaaaacaaattacaaaaattcaaaattttcgggtttattacaggga |
| | cagcagaaatccactttggaaaggaccagcaaagctcctctggaaaggtgaaggggcagtagtaatacaagataatagtgacataaaag |
| | tagtgccaagaagaaaagcaaagatcattagggattatggaaaacagatggcaggtgatgattgtgtggcaagtagacaggatgaggat |
| | tagaacatggaaaagtttagtaaaacaccata |
| 44 | ggtctctctggttagaccagatctgagcctgggagctctctggctaactagggaacccactgcttaagcctcaataaagcttgccttga |
| | gtgcttc |
| 45 | aagtagtgtgtgcccgtctgttgtgtgactctggtaactagagatccctcagacccttttagtcagtgtggaaaatctctagcag |
| 46 | ctgctttttgcctgtactg |
| 47 | atgggtgcgagagcgtcagtattaagcgggggagaattagatcgcgatgggaaaaaattcggttaaggccagggggaaagaaaaaatat |
| | aaattaaaacatatagtatgggcaagcagggagctagaacgattcgcagttaatactggcctgttagaaacatcagaaggctgtagaca |
| | aatactgggacagctacaaccatcccttcagacaggatcagaagaacttagatcattatataatacagtagcaaccctctattgtgtgc |
| | atcaaaggatagagataaaagacaccaaggaagctttagacaagatagaggaagagcaaaacaaaagtaagaaaaaagcacagcaagca |
| | gcag |
| 48 | ccggatgatcctgacgacggagtccgccgtcgtcgacaagccggcctggaagggctaattcactcccaaagaagacaagatagccccat |
| | cctcactgactccgtcctggagttggatgagagataatggccttacgttgtgccaggggagggtcgggctggatttagcaagatttacc |
| | ttctccaaagagcggtgctgcagtggcacagctgcccacggaggtgggggggtcaccgtccctggaggtgatgaagaactgtggggatg |
| | tggcactgagggacatggccagtgggcacggtgggtgggttggggttggtcttggggatcttggagggcttttccagccttcatgattt |
| | gacgattgtatgaacatctacatggcaattctccagctgcctgtcccagtcctactgacccagctgtatctctccaggcaagctcttcc |
| | accccttctgcttgcatccagacaccatcaaacatgcaggctcagacacatgatatcaagctttttccccgtatccccccaggtgtctg |
| | caggctcaaagagcagcgagaagcgttcagaggaaagcgatcccgtgccaccttccccgtgcccgggctgtccccgcacgctgccggct |
| | cggggatgcggggggagcgccggaccggagcggagccccgggcggctcgctgctgccccctagcgggggagggacgtaattacatccct |
| | gggggctttgggggggggctgtccccgtgagctccccagatctgctttttgcctgtactgggtctctctggttagaccagatctgagcc |
| | tgggagctctctggctaactagggaacccactgcttaagcctcaataaagcttgccttgagtgcttcaagtagtgtgtgcccgtctgtt |
| | gtgtgactctggtaactagagatccctcagacccttttagtcagtgtggaaaatctctagcagtggcgcccgaacagggacttgaaagc |
| | gaaagggaaaccagaggagctctctcgacgcaggactcggcttgctgaagcgcgcacggcaagaggcgaggggcggcgactggtgagta |
| | cgccaaaaattttgactagcggaggctagaaggagagagatgggtgcgagagcgtcagtattaagcgggggagaattagatcgcgatgg |
| | gaaaaaattcggttaaggccagggggaaagaaaaaatataaattaaaacatatagtatgggcaagcagggagctagaacgattcgcagt |
| | taatactggcctgttagaaacatcagaaggctgtagacaaatactgggacagctacaaccatcccttcagacaggatcagaagaactta |
| | gatcattatataatacagtagcaaccctctattgtgtgcatcaaaggatagagataaaagacaccaaggaagctttagacaagatagag |
| | gaagagcaaaacaaaagtaagaaaaaagcacagcaagcagcaggatcttcagacctggaaattccctacaatccccaaagtcaaggagt |
| | agtagaatctatgaataaagaattaaagaaaattataggacaggtaagagatcaggctgaacatcttaagacagcagtacaaatggcag |
| | tattcatccacaattttaaaagaaaaggggggattggggggtacagtgcaggggaaagaatagtagacataatagcaacagacatacaa |
| | actaaagaattacaaaaacaaattacaaaaattcaaaattttcgggtttattacagggacagcagaaatccactttggaaaggaccagc |
| | aaagctcctctggaaaggtgaaggggcagtagtaatacaagataatagtgacataaaagtagtgccaagaagaaaagcaaagatcatta |
| | gggattatggaaaacagatggcaggtgatgattgtgtggcaagtagacaggatgaggattagaacatggaaaagtttagtaaaacacca |
| | taaggaggagatatgagggacaattggagaagtgaattatataaatataaagtagtaaaaattgaaccattaggagtagcacccaccaa |
| | ggcaaagagaagagtggtgcagagagaaaaaagagcagtgggaataggagctttgttccttgggttcttgggagcagcaggaagcacta |
| | tgggcgcagcgtcaatgacgctgacggtacaggccagacaattattgtctggtatagtgcagcagcagaacaatttgctgagggctatt |
| | gaggcgcaacagcatctgttgcaactcacagtctggggcatcaagcagctccaggcaagaatcctggctgtggaaagatacctaaagga |
| | tcaacagctcctggggatttggggttgctctggaaaactcatttgcaccactgctgtgccttggaatgctagttggagtaataaatctc |
| | tggaacagatttggaatcacacgacctggatggagtgggacagagaaattaacaattacacaagcttaatacactccttaattgaagaa |
| | tcgcaaaaccagcaagaaaagaatgaacaagaattattggaattagataaatgggcaagtttgtggaattggtttaacataacaaattg |
| | gctgtggtatataaaattattcataatgatagtaggaggcttggtaggtttaagaatagtttttgctgtactttctatagtgaatagag |
| | ttaggcagggatattcaccattatgtttcagacccacctcccaaccccgaggggaccgagctcaagcttcgaagcgatcgcacgcgtgg |
| | atccgaacagagagacagcagaatatgggccaaacaggatatctgtggtaagcagttcctgccccggctcagggccaagaacagttgga |
| | acagcagaatatgggccaaacaggatatctgtggtaagcagttcctgccccggctcagggccaagaacagatggtccccagatgcggtc |
| | ccgccctcagcagtttctagagaaccatcagatgtttccagggtgccccaaggacctgaaatgaccctgtgccttatttgaactaacca |
| | atcagttcgcttctcgcttctgttcgcgcgcttctgctccccgagctctatataagcagagctcgtttagtgaaccgtcagatcggcgc |
| | gccaattcaagcgagaagacaagggcagccgccaccatggtgagcaagggcgaggccgtgatcaaggagttcatgaggttcaaggtgca |
| | catggagggctccatgaacggtcacgagttcgagatcgagggcgaaggcgagggcaggccctacgagggcacccagaccgccaagctga |
| | aggtgacaaagggcggccccctgcccttctcctgggacatcctgtcccctcagttcatgtacggctccagggccttcatcaagcacccc |
| | gccgacatccccgactactacaagcagtccttccccgagggcttcaagtgggagagggtgatgaacttcgaggacggcggcgccgtgac |
| | cgtgacccaggacacctccctggaggacggcaccctgatctacaaggtgaagctgaggggcaccaacttccctcctgacggccccgtga |
| | tgcagaagaagacaatgggctgggaggcctccaccgagaggctgtaccccgaggacggcgtgctgaagggcgacatcaagatggccctg |
| | aggctgaaggacggcggcagatacctggccgacttcaagaccacctacaaggccaagaagcccgtgcagatgcccggcgcctacaacgt |
| | ggacaggaagctggacatcacctcccacaacgaggactacaccgtggtggagcagtacgagaggtccgagggcaggcactccaccggcg |
| | gcatggacgagctgtataagtgactagtaatcaacctctggattacaaaatttgtgaaagattgactggtattcttaactatgttgctc |
| | cttttacgctatgtggatacgctgctttaatgcctttgtatcatgctattgcttcccgtatggctttcattttctcctccttgtataaa |
| | tcctggttgctgtctctttatgaggagttgtggcccgttgtcaggcaacgtggcgtggtgtgcactgtgtttgctgacgcaacccccac |
| | tggttggggcattgccaccacctgtcagctcctttccgggactttcgctttccccctccctattgccacggcggaactcatcgccgcct |
| | gccttgcccgctgctggacaggggctcggctgttgggcactgacaattccgtggtgttgtcggggaaatcatcgtcctttccttggctg |
| | ctcgcctgtgttgccacctggattctgcgcgggacgtccttctgctacgtcccttcggccctcaatccagcggaccttccttcccgcgg |
| | cctgctgccggctctgcggcctcttccgcgtcttcgccttcgccctcagacgagtcggatctccctttgggccgcctccccgcgcggcc |
| | gcatcgatgccgtagtacctttaagaccaatgacttacaaggcagctgtagatcttagccactttttaaaagaaaaggggggactggaa |
| | gggctaattcactcccaaagaagacaagatagccccatcctcactgactccgtcctggagttggatgagagataatggccttacgttgt |
| | gccaggggagggtcgggctggatttagcaagatttaccttctccaaagagcggtgctgcagtggcacagctgcccacggaggtgggggg |
| | gtcaccgtccctggaggtgatgaagaactgtggggatgtggcactgagggacatggccagtgggcacggtggggggttggggttggtct |
| | tggggatcttggagggcttttccagccttcatgatttgacgattgtatgaacatctacatggcaattctccagctgcctgtcccagtcc |
| | tactgacccagctgtatctctccaggcaagctcttccaccccttctgcttgcatccagacaccatcaaacatgcaggctcagacacatg |
| | atatcaagctttttccccgtatccccccaggtgtctgcaggctcaaagagcagcgagaagcgttcagaggaaagcgatcccgtgccacc |
| | ttccccgtgcccgggctgtccccgcacgctgccggctcggggatgcggggggagcgccggaccggagcggagccccgggcggctcgctg |
| | ctgccccctagcgggggagggacgtaattacatccctgggggctttgggggggggctgtccccgtgagctccccagatctgctttttgc |
| | ctgtactgggtctctctggttagaccagatctgagcctgggagctctctggctaactagggaacccactgcttaagcctcaataaagct |
| | tgccttgagtgcttcaagtagtgtgtgcccgtctgttgtgtgactctggtaactagagatccctcagacccttttagtcagtgtggaaa |
| | atctctagcagtccggatgatcctgacgacggagaccgccgtcgtcgacaagccggcctcgaggcatgcaagcttggcgtaatcatggt |
| | catagctgtttcctgtgtgaaattgttatccgctcacaattccacacaacatacgagccggaagcataaagtgtaaagcctggggtgcc |
| | taatgagtgagctaactcacattaattgcgttgcgctcactgcccgctttccagtcgggaaacctgtcgtgccagctgcattaatgaat |
| | cggccaacgcgcggggagaggcggtttgcgtattgggcgctcttccgcttcctcgctcactgactcgctgcgctcggtcgttcggctgc |
| | ggcgagcggtatcagctcactcaaaggcggtaatacggttatccacagaatcaggggataacgcaggaaagaacatgtgagcaaaaggc |
| | cagcaaaaggccaggaaccgtaaaaaggccgcgttgctggcgtttttccataggctccgcccccctgacgagcatcacaaaaatcgacg |
| | ctcaagtcagaggtggcgaaacccgacaggactataaagataccaggcgtttccccctggaagctccctcgtgcgctctcctgttccga |
| | ccctgccgcttaccggatacctgtccgcctttctcccttcgggaagcgtggcgctttctcatagctcacgctgtaggtatctcagttcg |
| | gtgtaggtcgttcgctccaagctgggctgtgtgcacgaaccccccgttcagcccgaccgctgcgccttatccggtaactatcgtcttga |
| | gtccaacccggtaagacacgacttatcgccactggcagcagccactggtaacaggattagcagagcgaggtatgtaggcggtgctacag |
| | agttcttgaagtggtggcctaactacggctacactagaagaacagtatttggtatctgcgctctgctgaagccagttaccttcggaaaa |
| | agagttggtagctcttgatccggcaaacaaaccaccgctggtagcggtggtttttttgtttgcaagcagcagattacgcgcagaaaaaa |
| | aggatctcaagaagatcctttgatcttttctacggggtctgacgctcagtggaacgaaaactcacgttaagggattttggtcatgagat |
| | tatcaaaaaggatcttcacctagatccttttaaattaaaaatgaagttttaaatcaatctaaagtatatatgagtaaacttggtctgac |
| | agttaccaatgcttaatcagtgaggcacctatctcagcgatctgtctatttcgttcatccatagttgcctgactccccgtcgtgtagat |
| | aactacgatacgggagggcttaccatctggccccagtgctgcaatgataccgcgagacccacgctcaccggctccagatttatcagcaa |
| | taaaccagccagccggaagggccgagcgcagaagtggtcctgcaactttatccgcctccatccagtctattaattgttgccgggaagct |
| | agagtaagtagttcgccagttaatagtttgcgcaacgttgttgccattgctacaggcatcgtggtgtcacgctcgtcgtttggtatggc |
| | ttcattcagctccggttcccaacgatcaaggcgagttacatgatcccccatgttgtgcaaaaaagcggttagctccttcggtcctccga |
| | tcgttgtcagaagtaagttggccgcagtgttatcactcatggttatggcagcactgcataattctcttactgtcatgccatccgtaaga |
| | tgcttttctgtgactggtgagtactcaaccaagtcattctgagaatagtgtatgcggcgaccgagttgctcttgcccggcgtcaatacg |
| | ggataataccgcgccacatagcagaactttaaaagtgctcatcattggaaaacgttcttcggggcgaaaactctcaaggatcttaccgc |
| | tgttgagatccagttcgatgtaacccactcgtgcacccaactgatcttcagcatcttttactttcaccagcgtttctgggtgagcaaaa |
| | acaggaaggcaaaatgccgcaaaaaagggaataagggcgacacggaaatgttgaatactcatactcttcctttttcaatattattgaag |
| | catttatcagggttattgtctcatgagcggatacatatttgaatgtatttagaaaaataaacaaataggggttccgcgcacatttcccc |
| | gaaaagtgccacctgacgtctaagaaaccattattatcatgacattaacctataaaaataggcgtatcacgaggccctttcgtctcgcg |
| | cgtttcggtgatgacggtgaaaacctctgacacatgcagctcccggagacggtcacagcttgtctgtaagcggatgccgggagcagaca |
| | agcccgtcagggcgcgtcagcgggtgttggcgggtgtcggggctggcttaactatgcggcatcagagcagattgtactgagagtgcacc |
| | atatgcggtgtgaaataccgcacagatgcgtaaggagaaaataccgcatcaggcgccattcgccattcaggctgcgcaactgttgggaa |
| | gggcgatcggtgcgggcctcttcgctattacgccagctggcgaaagggggatgtgctgcaaggcgattaagttgggtaacgccagggtt |
| | ttcccagtcacgacgttgtaaaacgacggccagtgaattc |
| 49 | ctcccgtgtggtacctgaggccggctcctgtggctctgagggggtctgcagcacccccttacatctgtccacagaagggctggggagca |
| | gctttcctgtccctcctgtgagtggccaccagggggagcgtggacacagctgcccgtgcagtgaccacctgccccccactcccgctact |
| | ccagcagcagcggctccagccctggacaccctccctgcccccaccagcctggtcctgagccaggtgacctcctccagcatcc |
| 50 | gtctgaatggtggccgtagtttgcagagccctggtttcttcttgcctctcagcttccaacttccccgtgagtgcctgctccttgatgga |
| | ctggactctaagcccttctttgcagcaagcacgatatcaagctttgtcagtagagggcgccggagggacactgtggaggaaggggcctt |
| | ttcatggtccacagagctctgttgtgcaatttcttgttcctgttgcatcttctcttagggtatgaacgcggggggacatcctctggggc |
| | ttttcctcagctgtgcacccagaatgcatggtccctcgaccacctcatagcccatcct |
| 51 | aggatatgcccttgactatttgtccgacatagtcaagggcatatcctttttt |
| 52 | aggatatgcccttgactatttgtccgacatagtcaagggcatatcc |
| 53 | tcgacgtgcagtatttagcatgccccacccatctgcaaggcattctggatagtgtcaaaacagccggaaatcaagtccgtttatctcaa |
| | actttagcattttgggaataaatgatatttgctatgctggttaaattagattttagttaaatttcctgctgaagctctagtacgataag |
| | taacttgacctaagtgtaaagttgagatttccttcaggtttatatagcttgtgcgccgcctgggtacctc |
| 54 | ctgcagtatttagcatgccccacccatctgcaaggcattctggatagtgtcaaaacagccggaaatcaagtccgtttatctcaaacttt |
| | agcattttgggaataaatgatatttgctatgctggttaaattagattttagttaaatttcctgctgaagctctagtacgataagcaact |
| | tgacctaagtgtaaagttgagatttccttcaggtttatatagcttgtgcgccgcctgggtacctc |
| 55 | Ctgtgccttctagttgccagccatctgttgtttgcccctcccccgtgccttccttgaccctggaaggtgccactcccactgtcctttcc |
| | taataaaatgaggaaattgcatcgcattgtctgagtaggtgtcattctattctggggggggggggggcaggacagcaagggggaggatt |
| | gggaagacaatagcaggcatgctggggatgcggtgggctctatgg |
| 56 | agccccatcctcactgactccgtcctggagttggatgagagataatggccttacgttgtgccaggggagggtcgggctggatttagcaa |
| | gatttaccttctccaaagagcggtgctgcagtggcacagctgcccacggaggtgggggggtcaccgtccctggaggtgatgaagaactg |
| | tggggatgtggcactgagggacatggccagtgggcacggtgggtgggttggggttggtcttggggatcttggagggcttttccagcctt |
| | catgatttgacgattgtatgaacatctacatggcaattctccagctgcctgtcccagtcctactgacccagctgtatctctccaggcaa |
| | gctcttccaccccttctgcttgcatcctgacaccatcaaacatgcaggctcagacacatgatatcaagctttttccccgtatcccccct |
| | ggtgtctgctggctcaaagagcagcgagaagcgttcagaggaaagcgatcccgtgccaccttccccgtgcccgggctgtccccgcacgc |
| | tgccggctcggggatgcggggggagcgccggaccggagcggagccccgggggctcgctgctgccccctagcgggggagggacgtaatta |
| | catccctgggggctttgggggggggctgtccccgtgagctc |
| 57 | agccccatcctcactgactccgtcctggagttggatgagagataatggccttacgttgtgccaggggagggtcgggctggatttagcaa |
| | gatttaccttctccaaagagcggtgctgcagtggcacagctgcccacggaggtgggggggtcaccgtccctggaggtgatgaagaactg |
| | tggggatgtggcactgagggacatggccagtgggcacggtgggtgggttggggttggtcttggggatcttggagggcttttccagcctt |
| | catgatttgacgattgtatgaacatctacatggcaattctccagctgcctgtcccagtcctactgacccagctgtatctctccaggcaa |
| | gctcttccaccccttctgcttgcatccagacaccatcaaacatgcaggctcagacacatgatatcaagctttttccccgtatcccccca |
| | ggtgtctgcaggctcaaagagcagcgagaagcgttcagaggaaagcgatcccgtgccaccttccccgtgcccgggctgtccccgcacgc |
| | tgccggctcggggatgcggggggagcgccggaccggagcggagccccgggcggctcgctgctgccccctagcgggggagggacgtaatt |
| | acatccctgggggctttgggggggggctgtccccgtgagctc |
| 58 | tgtgtctgagcctgcatgtttgatggtgtctggatgcaagcagaaggggtggaagagcttgcctggagagatacagctgggtcagtagg |
| | actgggacaggcagctggagaattgccatgtagatgttcatacaatcgtcaaatcatgaaggctggaaaagccctccaagatccccaag |
| | accaaccccaacccacccaccgtgcccactggccatgtccctcagtgccacatccccacagttcttcatcacctccagggacggtgacc |
| | cccccacctccgtgggcagctgtgccactgcagcaccgctctttggagaaggtaaatcttgctaaatccagcccgaccctcccctggca |
| | caacgtaaggccattatctctcatccaactccaggacggagtcagtgaggatggggct |
| 59 | gagctcacggggacagcccccccccaaagcccccagggatgtaattacgtccctcccccgctagggggcagcagcgagccgcccggggc |
| | tccgctccggtccggcgctccccccgcatccccgagccggcagcgtgcggggacagcccgggcacggggaaggtggcacgggatcgctt |
| | tcctctgaacgcttctcgctgctctttgagcctgcagacacctggggggatacggggaaaaagctt |
| 60 | ccttccctgccagagcttcggctgggggattcctccaccgcagcccccgagaaccacctggcctgctctgggcactgcttctgcagggg |
| | catggtgccatctggtggtgagactgggaactgcaccacagctccaaacggtacctgcacataagtcctggagactgaggggcaggcat |
| | ggagggattctgggggccctgcctttcctcacctgcccctggtctctccctccacccag |
| 61 | ccttcctttctaaatgacgagagagacagaagaattcttcaaggttagtgtgtccagcatgcaacctttccttcctggatgagcatccc |
| | tggaataggagagccagcctgcctcctgcgctggcacagagcccggttccctagacaactgcctctccaaatctgatgtccagcgccac |
| | ctggtgtccacatcaagcagacacaattaatagtcaacctgttcaggaaaactgtgagggggaaaaaaaagaaagaggatttatgaagg |
| | gaaaagaaagtttagaggatatgccacgattgg |
| 62 | acaatggctggcccatagtaaatgccgtgttagtgtgttagttgctgttcttccacgtcagaagaggcacagacaaattaccaccaggt |
| | ggcgctcagagtctgcggaggcatcacaacagccctgaatttgaatcctgctctgccactgcctagttgagaccttttactacctgact |
| | agctgagacatttacgacatttactggctctaggactcattttattcatttcattactttttttttctttgagacggaatctcgctct |
| 63 | gtgtagttcacacttatatcactagatgtctccctt |
| 64 | gactggacccctgccggcaggcggcagtgcaggcctgggtgctgccccctagcgggttagggacgtacatccctggg |
| 65 | actgtgcgacacagagtatcttttattcatccgtgtaagcaataagcaataacagaacagaacagaatgtttataataataacatttgt |
| | gattgtattcgcataggcatattagatcggatgatcgacaacaactacaaaaccggaatggggaagacagagagagagagagagagaga |
| | gagagaggggggggggaggaggggtggaggaggaggaggagagggggttgcgtctgagttgtagttgcagtaggagtttattatgaatt |
| | acagagactattatgaattacagagactttattctttcttgtatatttggtggccctgataagggccgttgaggtgttgaggtttgcga |
| | gtgcaaaacctttagcttgatttgccggtcttcttgggaagcagcacagcctggatgttgggcagtacaccacctcgagcgatggtcac |
| | acctccaagaagc |
| 66 | agccccatcctcactgactccgtcctggagttggatgagagataatggccttacgttgtgccaggggagggtcgggctggatttagcaa |
| | gatttaccttctccaaagagcggtgctgcagtggcacagctgcccacggaggtgggggggtcaccgtccctggaggtgatgaagaactg |
| | tggggatgtggcactgagggacatggccagtgggcacggtggggggttggggttggtcttggggatcttggagggcttttccagccttc |
| | atgatttgacgattgtatgaacatctacatggcaattctccagctgcctgtcccagtcctactgacccagctgtatctctccaggcaag |
| | ctcttccaccccttctgcttgcatccagacaccatcaaacatgcaggctcagacacagggaccagcagtgtctgtggcctttttgtgct |
| | cctctccatgctgggttttaacttgctctttgtccttctatcctatcttcttatccttaaggctgttctgaacgctgtgacttggagag |
| | tgtcccagagccctcaacacctgcatgtcccacgtccatgctgtcctgcacttccttatccccaagatctgcctctccgtgatgcactg |
| | aattggcaaacatgtgtcaccccagaccaacaatgtcacagcaaactcccccttgataggacaagggggaatggctttacactgagaca |
| | ggggaggtttgggttggatatgaggaggcagtttttcccccagagggtggtgacgcactgaacaggttgcccaaggaggctgtggatgc |
| | cccatccctgcaggcattcaaggccaggctggatgtggctctgggcagcctgggctgctggttgatgaccctgcacatagcagggggtt |
| | ggatctggatgagcactgtgctcctttgcaacccaggccgttctatgattctgtcattctaaatctctctttcagcctaaagctttttc |
| | cccgtatccccccaggtgtctgcaggctcaaagagcagcgagaagcgttcagaggaaagcgatcccgtgccaccttccccgtgcccggg |
| | ctgtccccgcacgctgccggctcggggatgcggggggagcgccggaccggagcggagccccgggcggctcgctgctgccccctagcggg |
| | ggagggacgtaattacatccctgggggctttgggggggggctgtccccgtgagctc |
| 67 | ggatgctggaggaggtcacctggctcaggaccaggctggtgggggcagggagggtgtccagggctggagccgctgctgctggagtagcg |
| | ggagtggggggcaggtggtcactgcacgggcagctgtgtccacgctccccctggtggccactcacaggagggacaggaaagctgctccc |
| | cagcccttctgtggacagatgtaagggggtgctgcagaccccctcagagccacaggagccggcctcaggtaccacacgggag |
| 68 | aggatgggctatgaggtggtcgagggaccatgcattctgggtgcacagctgaggaaaagccccagaggatgtccccccgcgttcatacc |
| | ctaagagaagatgcaacaggaacaagaaattgcacaacagagctctgtggaccatgaaaaggccccttcctccacagtgtccctccggc |
| | gccctctactgacaaagcttgatatcgtgcttgctgcaaagaagggcttagagtccagtccatcaaggagcaggcactcacggggaagt |
| | tggaagctgagaggcaagaagaaaccagggctctgcaaactacggccaccattcagac |
| 69 | atccctgcaggcattcaaggccaggctggatgtggctctgggcagcctgggctgctggttgatgaccctgcacatagcagggggttgga |
| | tctggatgagcactgtgctcctttgcaacccaggccgttctatgattctgtcattctaaatctctctttcagcctaaagctttttcccc |
| | gtatccccccaggtgtctgcaggctcaaagagcagcgagaagcgttcagaggaaagcgatcccgtgccaccttccccgtgcccgggctg |
| | tccccgcacgctgccggctcggggatgcggggggagcgccggaccggagcggagccccgggggctcgctgctgccccctagcgggggag |
| | ggacgtaattacatccctgggggctttgggggggggctgtccccgtgagctc |
| 70 | gagctcacggggacagcccccccccaaagcccccagggatgtaattacgtccctcccccgctagggggcagcagcgagccgcccggggc |
| | tccgctccggtccggcgctccccccgcatccccgagccggcagcgtgcggggacagcccgggcacggggaaggtggcacgggatcgctt |
| | tcctctgaacgcttctcgctgctctttgagcctgcagacacctggggggatacggggaaaaagctttaggctgaaagagagatttagaa |
| | tgacagaatcatagaacggcctgggttgcaaaggagcacagtgctcatccagatccaaccccctgctatgtgcagggtcatcaaccagc |
| | agcccaggctgcccagagccacatccagcctggccttgaatgcctgcagggat |

1. A landing pad cassette comprising:
a) a nucleotide sequence comprising a promoter operably linked to a nucleotide sequence encoding a detectable marker and/or a nucleotide sequence encoding a selection marker;
b) a nucleotide sequence encoding a suicide marker; and
c) a nucleotide sequence comprising a first site-specific recombination site and a nucleotide sequence comprising a second site-specific recombination site.

2. The landing pad cassette of claim 1, wherein the cassette comprises, in order from 5′ to 3′:
a) a nucleotide sequence comprising a first site-specific recombination site;
b) a nucleotide sequence comprising a promoter operably linked to a nucleotide sequence encoding a detectable marker and/or a nucleotide sequence encoding a selection marker;
c) a nucleotide sequence encoding a suicide marker; and
d) a nucleotide sequence comprising a second site-specific recombination site.

3. The landing pad cassette of claim 2, wherein:
a) the first and/or second site-specific recombination sites are attP recombination sites; and/or
b) the promoter is selected from the group consisting of a cytomegalovirus (CMV) promoter, a CMV enhancer, a murine leukemia virus-derived (MND) promoter, a simian virus 40 (SV40) promoter with enhancer, a polyubiquitin C gene (UBC) promoter, a phosphoglycerate kinase (PGK) promoter, a elongation factor-1 alpha (EF1A) promoter, a human β-actin (hACTB) promoter, a 7SK promoter, a cytomegalovirus immediate-early enhancer/chicken β-actin (CAG) promoter and combinations thereof; and/or
c) the detectable marker is selected from the group consisting of an enhanced green fluorescent protein (eGFP), a red fluorescent protein (mCherry or mScarlet), a yellow fluorescent protein, a cyan fluorescent protein or combinations thereof; and/or
d) the suicide marker is selected from the group consisting of Herpes Simplex Virus-1 thymidine kinase (HSV-TK), thymidine kinase (TK), caspase-9, caspase-8, purine nucleoside phosphorylase, uracil phosphoribosyl transferase, cytosine deaminase and combinations thereof; and/or
e) the selection marker is selected from the group consisting of a neomycin resistance gene, a hygromycin resistance gene, a puromycin N-acetyl-transferase, a histidinol dehydrogenase, a zeocin resistance gene, a bleomycin resistance gene, a blasticidin S deaminase and combinations thereof.

4. The landing pad cassette of claim 3, wherein:
a) the first and the second site-specific recombination sites are attP recombination sites; and/or
b) the nucleotide sequence comprising the CMV promoter comprises a sequence set forth in SEQ ID NO: 3 or 4, or the nucleotide sequence comprising the MND promoter comprises a sequence set forth in SEQ ID NO: 5; and/or
c) the nucleotide sequence encoding the eGFP comprises a sequence set forth in SEQ ID NO: 7; and/or
d) the nucleotide sequence encoding the HSV-TK comprises a sequence set forth in SEQ ID NO: 6; and/or
e) the nucleotide sequence encoding the puromycin N-acetyl-transferase comprises a sequence set forth in SEQ ID NO: 8.

5. The landing pad cassette of claim 2, further comprising:
a) a nucleotide sequence comprising a linker located between the nucleotide sequence encoding the detectable marker and the nucleotide sequence encoding the suicide marker, wherein the linker is an internal ribosome entry site (IRES) or encodes a 2A self-cleaving peptide, and wherein the 2A self-cleaving peptide is selected from the group consisting of a P2A, T2A, E2A and a F2A; and/or
b) a nucleotide sequence comprising a polyA signal located 3′ of the nucleotide sequence encoding the suicide marker, wherein the polyA signal is selected from the group consisting of a SV40 polyA, SVLP poly A, hGH poly A, BGH polyA and rbGlob poly A; and/or
c) a nucleotide comprising a woodchuck hepatitis virus post-transcriptional regulatory element (WPRE) located 3′ of the nucleotide sequence encoding the suicide marker.

6. The landing pad cassette of claim 5, wherein:
a) the nucleotide sequence encoding the P2A comprises a sequence set forth in any one of SEQ ID NOs: 10 to 13, or the nucleotide sequence encoding the T2A comprises a sequence set forth in SEQ ID NO: 15 or 16; and/or
b) the nucleotide sequence comprising the SV40 polyA signal comprises a sequence set forth in SEQ ID NO: 17;
c) the nucleotide sequence comprising the WPRE comprises a sequence set forth in SEQ ID NO: 20; and/or
d) the nucleotide sequence comprising the BGH polyA signal comprises a sequence set forth in SEQ ID NO: 55.

7. A landing pad cassette comprising, in order from 5′ to 3′:
a) a nucleotide sequence comprising an attP (GT) recombination site;
b) a nucleotide sequence comprising a CMV promoter or a nucleotide sequence comprising a MND promoter;
c) a nucleotide sequence encoding an enhanced green fluorescent protein (eGFP) detectable marker;
d) a nucleotide sequence encoding a P2A self-cleaving peptide linker;
e) a nucleotide sequence encoding a Herpes Simplex Virus-1 thymidine kinase (HSV-TK) suicide marker;
f) a nucleotide sequence comprising a WPRE;
g) a nucleotide sequence comprising a BGH polyA signal; and
h) a nucleotide sequence comprising an attP (GA) site.

8. A landing pad plasmid comprising the landing pad cassette of claim 1, comprising a nucleotide sequence comprising a 5′ homology arm (HA) and a nucleotide sequence comprising a 3′ HA.

9. The landing pad plasmid of claim 8, further comprising one or more of:
a) a nucleotide sequence comprising one or more additional promoters, wherein the one or more additional promoters is selected from the group consisting of a CMV promoter, a CMV enhancer, a MND promoter, a SV40 promoter with enhancer, a UBC promoter, a PGK promoter, a EFIA promoter, a hACTB promoter, a CAGG promoter and combinations thereof; and/or
b) a nucleotide sequence encoding one or more additional detectable markers, wherein the one or more additional detectable markers is selected from the group consisting of an enhanced green fluorescent protein (eGFP), a red fluorescent protein (mCherry or mScarlet), a yellow fluorescent protein, a cyan fluorescent protein and combinations thereof; and/or
c) a nucleotide sequence comprising an additional polyA signal, wherein the additional polyA signal is selected from the group consisting of a SV40 polyA, SVLP polyA, hGH polyA, BGH poly A and rbGlob polyA; and/or
d) a nucleotide sequence comprising a viral origin of replication sequence; and/or
e) a nucleotide sequence encoding an antibiotic resistance gene operably linked to a nucleotide sequence comprising a promoter.

10. The landing pad plasmid of claim 9, wherein:
a) the nucleotide sequence comprising the 5′ HA comprises a sequence set forth in SEQ ID NO: 24 or 26; and/or
b) the nucleotide sequence comprising the 3′ HA comprises a sequence set forth in SEQ ID NO: 25 or 27; and/or
c) the nucleotide sequence comprising the SV40 promoter with enhancer comprises a sequence set forth in SEQ ID NO: 17,
d) the nucleotide sequence comprising the BGH polyA signal comprises a sequence set forth in SEQ ID NO: 55.

11. The landing pad plasmid of claim 8, wherein the landing pad plasmid comprises a sequence set forth in SEQ ID NO: 1 or SEQ ID NO: 2.

12. A landing pad plasmid comprising, in order from 5′ to 3′:
a) a nucleotide sequence comprising a 5′ homology arm (HA);
b) a nucleotide sequence comprising an attP (GT) recombination site;
c) a nucleotide sequence comprising a CMV promoter or a nucleotide sequence comprising a MND promoter;
d) a nucleotide sequence encoding an enhanced green fluorescent protein (eGFP) detectable marker;
e) a nucleotide sequence encoding a P2A self-cleaving peptide linker;
f) a nucleotide sequence encoding an Herpes Simplex Virus-1 thymidine kinase (HSV-TK) suicide marker;
g) a nucleotide sequence comprising a WPRE;
h) a nucleotide sequence comprising a BGH polyA signal;
i) a nucleotide sequence comprising an attP (GA) recombination site;
j) a nucleotide sequence comprising a 3′ HA;
k) optionally a nucleotide sequence encoding a mCherry detectable marker;
l) a nucleotide sequence comprising a pUC viral origin of replication sequence; and
m) a nucleotide sequence encoding an Amp (R) gene operably linked to a nucleotide sequence comprising a bla promoter.

13. A method of stably integrating the landing pad cassette of claim 1 into the genome of a cell at a specific locus, wherein the landing pad is integrated at the specific locus using site-directed modification, and optionally further comprising selecting the cells comprising the landing pad plasmid.

14. The method of claim 13, wherein the specific locus is a leukemia oncogene (LMO2) locus, a MDS1 And EVIL Complex (MECOM) locus, a cyclin D2 (CCND2) locus, a B lymphoma Mo-MLV insertion region 1 homolog (BMI1) locus or a meningioma (disrupted in balanced translocation) 1 (MN1) locus, and optionally wherein the LMO2 locus is 33 kb upstream of the transcription start site (TSS) or 2 kb downstream of the TSS.

15. The method of claim 13, wherein selecting the cells comprising the landing pad cassette comprises selecting the cells resistant to antibiotic treatment by addition of an antibiotic, and optionally wherein the cells comprising the landing pad cassette are expanded to produce a stable cell line.

16. A stable cell line comprising the landing pad cassette of claim 1, preferably wherein the cell line is a Jurkat cell line or a K562 cell line.

17. A provirus construct comprising:
a) a nucleotide sequence comprising one of more site-specific recombination sites;
b) a nucleotide sequence comprising a 5′ long terminal repeat (LTR);
c) a nucleotide sequence encoding a transgene of interest operably linked to a promoter; and
d) a nucleotide sequence comprising a 3′ LTR comprising an insulator.

18. The provirus construct of claim 17, further comprising one or more of:
a) a nucleotide sequence encoding a suicide marker operably linked to a nucleotide sequence comprising a promoter; and/or
b) a nucleotide sequence comprising a polyA signal.

19. The provirus construct of claim 17 comprising, in order from 5′ to 3′:
a) a nucleotide sequence comprising a first site-specific recombination site, wherein the first site-specific recombination site is a attB (GT) recombination site;
b) a nucleotide sequence comprising a 5′ LTR;
c) a nucleotide sequence encoding a transgene of interest operably linked to a promoter, wherein the promoter is selected from the group consisting of a CMV promoter, a CMV enhancer, a MND promoter, a SV40 promoter with enhancer, a UBC promoter, a PGK promoter, a EF1A promoter, a hACTB promoter and a CAG promoter;
d) a nucleotide comprising a woodchuck hepatitis virus post-transcriptional regulatory element (WPRE);
e) a nucleotide sequence comprising a 3′ LTR comprising an insulator, wherein the insulator is a chicken hypersensitive site-4 (cHS4) insulator and optionally wherein the nucleotide sequence comprising the cHS4 insulator comprises a sequence set forth in any one of SEQ ID NOs: 29 to 31; and
f) a nucleotide sequence comprising a second site-specific recombination site, wherein the second site-specific recombination site is a attB (GA) recombination site.

20. A provirus construct comprising, in order from 5′ to 3′:
a) a nucleotide sequence comprising a attB (GT) recombination site;
b) a nucleotide sequence comprising a 5′ long terminal repeat (LTR);
c) a nucleotide sequence comprising lentiviral elements;
d) a nucleotide sequence comprising a MND promoter operably linked to a nucleotide sequence comprising a transgene of interest;
e) a nucleotide sequence comprising a woodchuck hepatitis virus post-transcriptional regulatory element (WPRE);
f) a nucleotide sequence comprising a 3′ LTR comprising a cHS4 insulator; and
g) a nucleotide sequence comprising a attB (GA) recombination site.

21. A provirus vector comprising the provirus construct of claim 17.

22. The provirus vector of claim 21, wherein the vector further comprises:
a) a nucleotide sequence encoding a suicide marker operably linked to a nucleotide sequence comprising a promoter, wherein the suicide marker is selected from the group consisting of a Herpes Simplex Virus-1 thymidine kinase (HSV-TK), a thymidine kinase (TK), a caspase-9, a caspase-8, a purine nucleoside phosphorylase, an uracil phosphoribosyl transferase or a cytosine deaminase and wherein the promoter is a CMV promoter, a CMV enhancer, a MND promoter, a SV40 promoter with enhancer, an UBC promoter, a PGK promoter, an EF1A promoter, a hACTB promoter and a CAG promoter; and
b) a nucleotide sequence comprising a polyA signal, wherein the polyA signal is selected from the group consisting of a SV40 polyA, SVLP polyA, hGH polyA, BGH polyA or rbGlob polyA.

23. The provirus vector of claim 22, wherein the vector comprises a nucleotide sequence comprising a CMV promoter operably linked to a nucleotide sequence encoding a Herpes Simplex Virus-1 thymidine kinase (HSV-TK) suicide marker; and a nucleotide sequence comprising a SV40 polyA signal.

24. The provirus vector of claim 21, wherein the provirus vector comprises a sequence set forth in SEQ ID NO: 48.

25. A method of stably integrating the provirus construct of claim 17 into the stable cell line of claim 16, wherein the provirus construct is integrated between the first and the second site-specific recombination sites present in the stable cell line.

26. The method of claim 25, wherein the method comprises transfecting the provirus construct into the cell line in a cell culture in the presence of a recombinase, wherein the recombinase and provirus construct are added to the cell culture at a ratio of at least 3 to 1, and optionally wherein the recombinase is a serine recombinase Bxb1.

27. The method of claim 25, wherein the method further comprises selecting the cells comprising the integrated provirus construct by addition of a compound that activates the suicide marker in the cells that do not have the integrated provirus construct, optionally wherein the compound is ganciclovir (GCV).

28. A stable cell line comprising the provirus construct of claim 17.

29. Use of the stable cell line of claim 28 for production of an enveloped virus.

30. A method of developing an improved cell line for use as a screening tool for components of a modified provirus construct component.

31. A method of optimising a provirus construct, comprising integrating a provirus construct into a cell line comprising the landing pad cassette of claim 1, and assessing the activity of the integrated construct.

32. A method for assessing safety, genotoxicity and/or efficacy of a provirus construct, comprising:
a) integrating the landing pad cassette of claim 1 into the genome of a cell at a specific locus;
b) selecting the cells comprising the landing pad cassette, wherein selecting comprises selecting the cells resistant to antibiotic treatment by addition of an antibiotic;
c) expanding the cells comprising the landing pad cassette to produce a stable cell line;
d) integrating the provirus construct into the stable cell line of step c), wherein the provirus construct is integrated between the first and the second site-specific recombination sites present in the stable cell line;
e) selecting the cells comprising the integrated provirus construct, wherein selecting the cells comprising the integrated provirus construct comprises adding a compound to activate the suicide marker in the cells that do not have the integrated provirus construct;
f) expanding the cells comprising the provirus construct to produce a final cell line; and
g) measuring the expression of the locus to determine the safety, genotoxicity and/or efficacy of the provirus or components of the provirus.

33. The method of claim 32, wherein the method further comprises comparing the safety, genotoxicity and/or efficacy of at least two proviruses or modified provirus construct components to identify an optimal provirus or modified provirus construct component.

34. The method of claim 32, wherein the component of the provirus is an insulator, a promoter, an enhancer, a lentiviral element or combinations thereof, preferably wherein the component is an insulator.

35. The method of claim 32, wherein the provirus construct is a construct of claim 17.

36. The method of claim 32, wherein the specific locus is a leukemia oncogene (LMO2) locus, a MDS1 And EVI1 Complex (MECOM) locus, a cyclin D2 (CCND2) locus, a B lymphoma Mo-MLV insertion region 1 homolog (BMI1) locus or a meningioma (disrupted in balanced translocation) 1 (MN1) locus, and optionally wherein the LMO2 locus is 33 kb upstream of the transcription start site (TSS) or 2 kb downstream of the TSS.
